SEQUENCE LISTING (104 sequences)

SEQ ID NO: 1 - protein, 22 aa, Artificial Sequence
  signal peptide
MLLLVTSLLLCELPHPAFLLIP

SEQ ID NO: 2 - protein, 254 aa, Artificial Sequence
  Fluorescein scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 3 - protein, 12 aa, Artificial Sequence
  linker
ESKYGPPCPPCP

SEQ ID NO: 4 - protein, 28 aa, Artificial Sequence
  transmembrane span
MFWVLVVVGGVLACYSLLVTVAFIIFWV

SEQ ID NO: 5 - protein, 154 aa, Artificial Sequence
  4-1BB signaling domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 22 aa, Artificial Sequence
  self-cleaving peptide
GSGATNFSLLKQAGDVEENPGP

SEQ ID NO: 7 - protein, 266 aa, Artificial Sequence
  CD19 scFV
METDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 8 - protein, 17 aa, Artificial Sequence
  linker
ASGGGGSGGGGSGGGGS

SEQ ID NO: 9 - protein, 35 aa, Artificial Sequence
  CD4 TM
GGRMALIVLGGVAGLLLFIGLGIFFCVRCRHRRRQ

SEQ ID NO: 10 - protein, 1030 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGGGGSGGGGSGSSRTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSESKYGPPCPPCPGGRMALIVLGGVAGLLLFIGLGIFFCVRCRHRRRQ

SEQ ID NO: 11 - protein, 983 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGGGGSGGGGSGSSRTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMS

SEQ ID NO: 12 - protein, 1079 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGGGGSGGGGSHHWRYGGDPPWPRVSPACAGRFQSPVDIRPQLAAFSPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDESKYGPPCPPCPGGRMALIVLGGVAGLLLFIGLGIFFCVRCRHRRRQ

SEQ ID NO: 13 - protein, 1044 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGGGGSGGGGSHHWRYGGDPPWPRVSPACAGRFQSPVDIRPQLAAFSPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDESKYGPPCPPCP

SEQ ID NO: 14 - protein, 1078 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGGGGSGGGGSHWRYGGDPPWPRVSPACAGRFQSPVDIRPQLAAFSPALRPLELSGFQLPPLPELRLRNNGHSVQLTLPPGLEMKLGPGREYRALQLHLHWGAAGRPGSEHTVEGHRFPAEIHVVHLSTKYARVDEALGRPGGLAVLAAFLEEGPEENSAYEQLLSRLEEIAEEGSETQVPGLDISALLPSDFSRYFQYEGSLTTPPCAQGVIWTVFNQTVSLSAKQLHTLSDTLWGPGDSRLQLNFRATQPLNGRVIEASFPAGVDESKYGPPCPPCPGGRMALIVLGGVAGLLLFIGLGIFFCVRCRHRRRQ

SEQ ID NO: 15 - protein, 1043 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGGGGSGGGGSHWRYGGDPPWPRVSPACAGRFQSPVDIRPQLAAFSPALRPLELSGFQLPPLPELRLRNNGHSVQLTLPPGLEMKLGPGREYRALQLHLHWGAAGRPGSEHTVEGHRFPAEIHVVHLSTKYARVDEALGRPGGLAVLAAFLEEGPEENSAYEQLLSRLEEIAEEGSETQVPGLDISALLPSDFSRYFQYEGSLTTPPCAQGVIWTVFNQTVSLSAKQLHTLSDTLWGPGDSRLQLNFRATQPLNGRVIEASFPAGVDESKYGPPCPPCP

SEQ ID NO: 16 - protein, 919 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEESKYGPPCPPCPGGRMALIVLGGVAGLLLFIGLGIFFCVRCRHRRRQ

SEQ ID NO: 17 - protein, 887 aa, Artificial Sequence
  GATR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEESKYGPPCPPCPGGR

SEQ ID NO: 18 - protein, 208 aa, Artificial Sequence
  FRalpha
GSSRTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMS

SEQ ID NO: 19 - protein, 257 aa, Artificial Sequence
  CA9 domain
HHWRYGGDPPWPRVSPACAGRFQSPVDIRPQLAAFSPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVD

SEQ ID NO: 20 - protein, 256 aa, Artificial Sequence
  CA9 Domain
HWRYGGDPPWPRVSPACAGRFQSPVDIRPQLAAFSPALRPLELSGFQLPPLPELRLRNNGHSVQLTLPPGLEMKLGPGREYRALQLHLHWGAAGRPGSEHTVEGHRFPAEIHVVHLSTKYARVDEALGRPGGLAVLAAFLEEGPEENSAYEQLLSRLEEIAEEGSETQVPGLDISALLPSDFSRYFQYEGSLTTPPCAQGVIWTVFNQTVSLSAKQLHTLSDTLWGPGDSRLQLNFRATQPLNGRVIEASFPAGVD

SEQ ID NO: 21 - protein, 111 aa, Artificial Sequence
  FRB
EMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGP

SEQ ID NO: 22 - protein, 21 aa, Artificial Sequence
  signal sequence
MALPVTALLLPLALLLHAARP

SEQ ID NO: 23 - protein, 93 aa, Artificial Sequence
  DmrC
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 24 - protein, 27 aa, Artificial Sequence
  transmembrane
AGTGSDIYIWAPLAGTCGVLLLSLVIT

SEQ ID NO: 25 - protein, 42 aa, Artificial Sequence
  4-1BB
KRGRKKLLYIFKQPFMRPVQTTQEEDGCSCRFPEEEEGGCEL

SEQ ID NO: 26 - protein, 112 aa, Artificial Sequence
  zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 19 aa, Artificial Sequence
  P2A
ATNFSLLKQAGDVEENPGP

SEQ ID NO: 28 - protein, 20 aa, Artificial Sequence
  signal sequence
METDTLLLWVLLLWVPGSTG

SEQ ID NO: 29 - protein, 245 aa, Artificial Sequence
  CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 254 aa, Artificial Sequence; identical to SEQ ID NO: 2
  Fluorescein scFV

SEQ ID NO: 31 - protein, 110 aa, Artificial Sequence [sequence omitted]

SEQ ID NO: 32 - protein, 127 aa, Artificial Sequence
  Fluorescein heavy chain
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSS

SEQ ID NO: 33 - protein, 251 aa, Artificial Sequence
  Fluorescein scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 34 - protein, 19 aa, Artificial Sequence
  stalk domain
ESKYGPPCPPCPAPEFDGG

SEQ ID NO: 35 - protein, 210 aa, Artificial Sequence
  stalk domain
PSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 23 aa, Artificial Sequence
  T2A
LEGGGEGRGSLLTCGDVEENPGP

SEQ ID NO: 37 - protein, 358 aa, Artificial Sequence
  tEGFR
RMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 38 - protein, 5 aa, Artificial Sequence
  G4S
GGGGS

SEQ ID NO: 39 - protein, 107 aa, Artificial Sequence
  FKBP
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 40 - protein, 22 aa, Artificial Sequence
  transmembrane
MALIVLGGVAGLLLFIGLGIFF

SEQ ID NO: 41 - protein, 10 aa, Artificial Sequence
  CD4IC
CVRCRHRRRQ

SEQ ID NO: 42 - protein, 12 aa, Artificial Sequence
  linker
ESKYGPPCPPCP

SEQ ID NO: 43 - protein, 20 aa, Artificial Sequence
  linker
ESKYGPPCPPCPPAPEFDGG

SEQ ID NO: 44 - protein, 45 aa, Artificial Sequence
  stalk domain
TTTPAPRPPTPAPTIASQPLSLRPEACRPAAGGAVHTRGLDFACD

SEQ ID NO: 45 - protein, 24 aa, Artificial Sequence
  transmembrane
IYIWAPLAGTCGVLLLSLVITLYC

SEQ ID NO: 46 - protein, 109 aa, Artificial Sequence
  ABT-806 VL domain
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRA

SEQ ID NO: 47 - protein, 120 aa, Artificial Sequence
  ABT-806 light chain
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPP

SEQ ID NO: 48 - protein, 116 aa, Artificial Sequence
  ABT-806 VH Domain
EVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSS

SEQ ID NO: 49 - protein, 115 aa, Artificial Sequence
  ABT-806 heavy chain
EVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPWGQGTLVTVSS

SEQ ID NO: 50 - protein, 115 aa, Artificial Sequence
  ABT-806 heavy chain
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPWGQGTLVTVSS

SEQ ID NO: 51 - protein, 240 aa, Artificial Sequence
  ABT-806 scFv
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRSTSGSGKPGSGEGSTKGEVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPWGQGTLVTVSS

SEQ ID NO: 52 - protein, 245 aa, Artificial Sequence; identical to SEQ ID NO: 29
  CD19-CAR

SEQ ID NO: 53 - protein, 8 aa, Artificial Sequence
  CD19 CAR - CDRL1
QDISKYLN

SEQ ID NO: 54 - protein, 11 aa, Artificial Sequence
  CD19 CAR - CDRL2
LLIYHTSRLHS

SEQ ID NO: 55 - protein, 8 aa, Artificial Sequence
  CD19 CAR - CDRL3
QQGNTLPY

SEQ ID NO: 56 - protein, 446 aa, Artificial Sequence
  Depatuxizumab - heavy chain
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 214 aa, Artificial Sequence
  ABT-806 - light chain
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 448 aa, Artificial Sequence
  GATC - CAR polypeptide
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGSTSGSGKPGSGEGSTKGQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 255 aa, Artificial Sequence
  GATR - CAR polypeptide
GSSRTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSESKYGPPCPPCPGGRMALIVLGGVAGLLLFIGLGIFFCVRCRHRRRQ

SEQ ID NO: 60 - protein, 304 aa, Artificial Sequence
  GATR - CAR polypeptide
HHWRYGGDPPWPRVSPACAGRFQSPVDIRPQLAAFSPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDESKYGPPCPPCPGGRMALIVLGGVAGLLLFIGLGIFFCVRCRHRRRQ

SEQ ID NO: 61 - protein, 269 aa, Artificial Sequence
  CA9 domain and linker
HHWRYGGDPPWPRVSPACAGRFQSPVDIRPQLAAFSPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDESKYGPPCPPCP

SEQ ID NO: 62 - protein, 5 aa, Artificial Sequence
  CD19 CAR-CDRH1
SYWMN

SEQ ID NO: 63 - protein, 17 aa, Artificial Sequence
  CD19 CAR-CDRH2
QIWPGDGDTNYNGKFKG

SEQ ID NO: 64 - protein, 15 aa, Artificial Sequence
  CD19 CAR-CDRH3
RETTTVGRYYYAMDY

SEQ ID NO: 65 - protein, 511 aa, Artificial Sequence
  Cocal G protein
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 66 - protein, 7 aa, Artificial Sequence
  OKT3 - CDRH1
GYTFTRY

SEQ ID NO: 67 - protein, 6 aa, Artificial Sequence
  OKT3 - CDRH2
NPSRGY

SEQ ID NO: 68 - protein, 10 aa, Artificial Sequence
  OKT3 - CDRH3
YYDDHYCLDY

SEQ ID NO: 69 - protein, 10 aa, Artificial Sequence
  OKT3 - CDRL1
SASSSVSYMN

SEQ ID NO: 70 - protein, 7 aa, Artificial Sequence
  OKT3 - CDRL2
DTSKLAS

SEQ ID NO: 71 - protein, 9 aa, Artificial Sequence
  OKT3 - CDRL3
QQWSSNPFT

SEQ ID NO: 72 - protein, 7 aa, Artificial Sequence
  15E8 - CDRH1
GFSLTSY

SEQ ID NO: 73 - protein, 5 aa, Artificial Sequence
  15E8 - CDRH2
WAGGS

SEQ ID NO: 74 - protein, 15 aa, Artificial Sequence
  15E8 - CDRH3
DKRAPGKLYYGYPDY

SEQ ID NO: 75 - protein, 15 aa, Artificial Sequence
  15E8 - CDRL1
RASESVEYYVTSLMQ

SEQ ID NO: 76 - protein, 7 aa, Artificial Sequence
  15E8 - CDRL2
AASNVES

SEQ ID NO: 77 - protein, 9 aa, Artificial Sequence
  15E8 - CDRL3
QQTRKVPST

SEQ ID NO: 78 - protein, 6 aa, Artificial Sequence
  TGN1412 - CDRH1
GYTFSY

SEQ ID NO: 79 - protein, 6 aa, Artificial Sequence
  TGN1412 - CDRH2
YPGNVN

SEQ ID NO: 80 - protein, 11 aa, Artificial Sequence
  TGN1412 - CDRH3
SHYGLDWNFDV

SEQ ID NO: 81 - protein, 10 aa, Artificial Sequence
  TGN1412 - CDRL1
HASQNIYVLN

SEQ ID NO: 82 - protein, 7 aa, Artificial Sequence
  TGN1412 - CDRL2
KASNLHT

SEQ ID NO: 83 - protein, 9 aa, Artificial Sequence
  TGN1412 - CDRL3
QQGQTYPYT

SEQ ID NO: 84 - protein, 173 aa, Artificial Sequence
  OX40L
MERVQPLEENVGNAARPRFERNKLLLVASVIQGLGLLLCFTYICLHFSALQVSHRYPRIQSIKVQFTEYKKEKGFILTSQKEDEIMKVQNYLISLKGYFSQEVNISLHYQKDEEPLFQLKKVRSVNSLMVASLTYKDKVYLNVTTDNTSLDDFHVNGGELILIHQNPGEFCVL

SEQ ID NO: 85 - protein, 254 aa, Artificial Sequence
  4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 86 - protein, 233 aa, Artificial Sequence
  hinge region of lgG1
AEPKSPDKTHTCPPCPAPPVAGPSVFLFPPKPKDTLMIARTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 87 - protein, 46 aa, Artificial Sequence
  stalk domain
TTTPAPRPPTPAPTIASQPLSLRPEACRPAAGGAVHTRGLDFACDI

SEQ ID NO: 88 - protein, 20 aa, Artificial Sequence
  IgG1 hinge region
AEPKSPDKTHTCPPCPKDPK

SEQ ID NO: 89 - protein, 185 aa, Artificial Sequence
  ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 90 - protein, 259 aa, Artificial Sequence
  ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 91 - protein, 153 aa, Artificial Sequence
  IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 92 - protein, 177 aa, Artificial Sequence
  IL7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 93 - protein, 162 aa, Artificial Sequence
  IL15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 94 - protein, 271 aa, Artificial Sequence
  IL7
MAHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 95 - protein, 256 aa, Artificial Sequence
  IL-15
MGLVRRGARAGPRMPRGWTALCLLSLLPSGFMAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 96 - protein, 4 aa, Artificial Sequence
  G4S
GGGS

SEQ ID NO: 97 - protein, 7 aa, Artificial Sequence
  linker
GGGSGGG

SEQ ID NO: 98 - protein, 3 aa, Artificial Sequence
  linker
GGG

SEQ ID NO: 99 - protein, 10 aa, Artificial Sequence
  Fluorescein - CDRL1
TSNIGNNYVS

SEQ ID NO: 100 - protein, 11 aa, Artificial Sequence
  Fluorescein - CDRL2
LMIYDVSKRPS

SEQ ID NO: 101 - protein, 10 aa, Artificial Sequence
  Fluorescein - CDRL3
AAWDDSLSEF

SEQ ID NO: 102 - protein, 9 aa, Artificial Sequence
  Fluorescein - CDRH1
FTFGSFSMS

SEQ ID NO: 103 - protein, 14 aa, Artificial Sequence
  Fluorescein - CDRH2
WVAGLSARSSLTHY

SEQ ID NO: 104 - protein, 19 aa, Artificial Sequence
  Fluorescein - CDRH3
RRSYDSSGYWGHFYSYMDV

(19) United States
(12) Patent Application Publication
(10) Pub. No.: US 2023/0272039 A1
(43) Pub. Date: Aug. 31, 2023

(54) GATED ADAPTER TARGETING RECEPTOR

(71) Applicant: Umoja Biopharma, Inc., Seattle, WA (US)

(72) Inventors: Andrew SCHARENBERG, Seattle, WA (US); Laurie BEITZ, Seattle, WA (US)

(73) Assignee: Umoja Biopharma, Inc., Seattle, WA (US)

(21) Appl. No.: 18/016,307

(22) PCT Filed: Jul. 15, 2021
(86) PCT No.: PCT/US2021/041789

§ 371 Date: Jan. 13, 2023

Related U.S. Application Data
(60) Provisional application No. 63/052,806, filed on Jul. 16, 2020.

(51) Int. Cl. C07K 14/725; C07K 16/28; A61K 47/55; C12N 15/63

(52) U.S. Cl. CPC C07K 14/7051; C07K 16/28; A61K 47/551; C12N 15/63; C07K 2317/622; C07K 2319/03

(57) ABSTRACT

Provided are compositions and methods for polynucleotides and vectors encoding novel chimeric receptor systems. The Gated Adaptor Targeting Receptor (GATR) system employs a dual adaptor system: a first, targeting adaptor and a second, gating adaptor. The targeting adaptor bispecifically binds both the target cell and the gating adaptor, while an engineered cell expressing the chimeric receptor binds the gating adaptor. This targets the engineered cells to an antigen recognized by the targeting adaptor thereby activating the engineered cells and leading to the desired physiological effect.

### CROSS-REFERENCE TO RELATED APPLICATIONS

This application claims the benefit of U.S. Provisional Application No. 63/052,806, filed on Jul. 16, 2020, the content of which is herein incorporated by reference in its entirety.

### INCORPORATION BY REFERENCE OF SEQUENCE LISTING

The instant application contains a Sequence Listing which has been submitted in ASCII format via EFS-Web and is hereby incorporated by reference in its entirety. Said ASCII copy, created on Jul. 5, 2021, is named “UMOJ_006_01WO_SeqList_ST25.txt” and is about 160 KB in size.

### TECHNICAL FIELD

The technical field of this invention is polynucleotides, vectors, cells and method of use in engineering immune cells and treating disease. More specifically, the invention relates to polynucleotides and vectors encoding novel chimeric receptor systems and related compositions and methods.

### BACKGROUND

Chimeric receptors are engineered receptors used to genetically engineer T cells for use in adoptive cellular immunotherapy (Pule et al., Cytother. 5:3, 2003; Restifo et al., Nat. Rev. Immunol. 12:269, 2012). These receptors include an extracellular ligand binding domain, most commonly a single chain variable fragment of a monoclonal antibody (scFv), linked to intracellular signaling components, most commonly CD3ζ alone or combined with one or more costimulatory domains. T-cell receptor (TCR)-based chimeric receptors employ a single-chain fusion of the TCR Vα, Cα, Vβ, and Cβ domains in place of the scFv (see Walseng et al. Sci. Rep. 7:10713 (2017). Antigen binding stimulates the signaling domains on the intracellular segment of the chimeric receptor, thereby activating signaling pathways. chimeric receptor-based adoptive cellular immunotherapy has been used to treat cancer patients with tumors refractory to conventional standard-of-care treatments (see Grupp et al., N. Engl. J. Med. 368:1509, 2013; Kalos et al., Sci. Transl. Med. 3:95ra73, 2011). T regulatory cells transduced with a chimeric antigen receptor (CAR) (CAR T_{regs}) can be used to induce immune tolerance (Zhang et al. Front. Immunol. 9:2359 (2018). CAR NK cells can also be made (Mehta et al. Front. Immunol. 9:283 (2018). The costimulatory domains may be replaced with inhibitory domains to generate an inhibitory CAR (iCAR) (Federov et al. Sci. Trans. Med. 5:215ra172 (2013) and WO 2015/142314 A1).

Most CAR-based therapies rely upon specific binding of the CAR to cell-surface antigens already present on target cells. However, U.S. Pat. No. 9,233,125 provides a universal CAR having an extracellular ligand binding domain that binds to a tag; the tag is conjugated to an antibody; and this tagged antibody recognizes a cell-surface antigen—for example a tumor antigen. The tagged antibody serves as an adaptor for the CAR. Thus, “universal” CAR can be used to target various cell-surface antigens using interchangeable tagged antibodies.

WO 2014/100615 extends this adaptor concept to other tagged molecules (termed small conjugate molecules) capable of binding to cells. In place of a tagged antibody, the tagged molecule may be a small molecule that binds tumor cells—such as folate, 2-3-(1,3-dicarboxypropyl)ureido pentanedioic acid (DUPA), or cholecystokinin 2 receptor (CCK2R) ligand.

WO2019156795A1 discloses use of a tagged lipid, rather than a tagged antibody, to target the universal CAR to cells. The tag may optionally be masked with chemically liable protecting group. Demasking of the tag by reactive oxygen species present in the tumor microenvironment activates the tagged lipid within the tumor.

There remains an unmet need for polynucleotides, vectors, and cells for immunotherapy.

### SUMMARY OF THE INVENTION

The Gated Adaptor Targeting Receptor (GATR) system described here employs dual adaptors to extend the utility of universal chimeric receptors. Rather than a tagged antibody or tagged lipid, the GATR system uses a dual adaptor system: a first, targeting adaptor and a second, gating adaptor. The targeting adaptor bispecifically binds both the target cell and the gating adaptor. The chimeric receptor in turn recognizes the gating adaptor. Engineered cells expressing the chimeric receptor bind the gating adaptor. This targets the engineered cells to an antigen recognized by the targeting adaptor. Binding of the chimeric receptor-expressing engineered cell to an antigen-expressing target cell activates (or represses with an iCAR) the immunological activated of the engineered cell.

Thus, when the components of the GATR system—the immune cell expressing the chimeric receptor, the targeting adaptor, and the gating adaptor—are all provided at effective concentrations at an etiologically relevant site in the subject, they form a multi-part receptor that activates the engineered cell, leading to the desired physiological effect. The targeting adaptor may be a polypeptide, such as one lacking any chemical conjugation—e.g., a polypeptide expressed in the subject from a vector. The gating adaptor may be a small molecule—e.g., a soluble small molecule having distinct moieties recognized by the chimeric receptor and the targeting adaptor, respectively.

The present disclosure provides a Gated Adaptor Targeting Receptor (GATR) system, comprising: (a) a gating adaptor; (b) a targeting adaptor, or a vector encoding the targeting adaptor; and (c) an engineered immune cell comprising a chimeric receptor, or a vector encoding the chimeric receptor, wherein the targeting adaptor comprises a first ligand-binding domain (tLBD-1) specific for a cell-surface antigen and a second ligand-binding domain (tLBD-2) specific for the gating adaptor; and wherein the chimeric receptor comprises an extracellular ligand-binding domain (rLBD) specific for the gating adaptor, a transmembrane domain, and an intracellular actuator domain.

Advantages of some embodiments may include achieving (1) inducible activation of the immune cell by controlled administration of the gating adaptor; (2) titrated response by controlled administration of the gating adaptor; (3) use of an off-the-self universal CAR, e.g. CARs developed for or suitable for use with single adaptor systems; (4) use of the same universal CAR for different disease indications; (5) generation of engineered immune cells by in vivo transduction; (6) generation of the targeting adaptor by in vivo transduction; and/or (7) targeting of chimeric receptor immune cells to multiple targets by administering several targeting adaptors.

The foregoing advantages may be present in some but not all of the embodiments described herein, and other advantages of the GATR system will become apparent from the detailed description that follows.

### BRIEF DESCRIPTION OF FIGURES

FIG. 1 is a diagram depicting the GATR system wherein the cellular membrane of an engineered cell is spanned from extracellular to intracellular by a chimeric receptor. A gating adaptor binds both the chimeric receptor and the targeting adaptor. This system targets an engineered cell to a target cell.

FIG. 2 is a diagram depicting the GATR system wherein the cellular membrane of an engineered cell is spanned from extracellular to intracellular by a chimeric receptor. A gating adaptor binds both the chimeric receptor and the targeting adaptor which comprises a transmembrane tether that tethers the targeting adaptor to the engineered cell. This system targets an engineered cell to a target cell.

FIG. 3 is a diagram depicting the GATR system wherein the cellular membrane of an engineered cell is spanned from extracellular to intracellular by a chimeric receptor. The engineered cell may be used for targeting a cell via a small molecule that is recognized by a receptor to a targeting cell.

FIG. 4 is a diagram depicting the GATR system wherein the cellular membrane of an engineered cell is spanned from extracellular to intracellular by a chimeric receptor. The engineered cell may be used for targeting a cell via a membrane-binding small molecule on a targeting cell.

FIG. 5 is a diagram depicting the GATR system wherein the cellular membrane of an engineered cell is spanned from extracellular to intracellular by a chimeric receptor. The chimeric receptor or the targeting adaptor may include a CID-based multimerization domain pair which may be inserted in the targeting adaptor.

FIG. 6 is a diagram depicting the GATR system wherein the cellular membrane of an engineered cell is spanned from extracellular to intracellular by a chimeric receptor. The chimeric receptor or the targeting adaptor may include a CID-based multimerization domain pair, which may be inserted in the extracellular portion of the chimeric receptor.

FIG. 7 is a diagram depicting the GATR system wherein the cellular membrane of an engineered cell is spanned from extracellular to intracellular by a chimeric receptor. The chimeric receptor or the targeting adaptor may include a CID-based multimerization domain pair, which may be inserted in the intracellular portion of the chimeric receptor.

FIG. 8 is a diagram depicting the GATR system wherein the cellular membrane of an engineered cell is spanned from extracellular to intracellular by a chimeric receptor. A gating adaptor binds both the chimeric receptor and the targeting adaptor and is a macromolecule.

FIG. 9 is a diagram depicting the GATR system wherein the cellular membrane of an engineered cell is spanned from extracellular to intracellular by a chimeric receptor. A gating adaptor binds both the chimeric receptor and the targeting adaptor and is a small molecule conjugate of a macromolecule.

FIG. 10 is a diagram depicting embodiments of the GATR system encoding two membrane proteins: an anti-fluorescein scFv fused via a transmembrane domain to a 41bb-zeta intracellular domain; and an anti-CD19 scFv fused to a CA9 domain in turn fused to a transmembrane domain. A fluorescein-acetazolamide (“FITC-Aza”) is used to gate binding of the two proteins.

FIG. 11 is a graph depicting IFNγ cytokine production in response to an increasing concentration of gating adaptor FITC-Aza in control (CD19-K562) and target (CD19+K562) cells following 12 or 72 hours of co-culture.

FIG. 12 is a graph depicting IL-2 cytokine production in response to an increasing concentration of gating adaptor FITC-Aza in control (CD19-K562) and target (CD19+K562) cells following 12 or 72 hours of co-culture.

FIG. 13 is a diagram depicting embodiments of the GATR system encoding two membrane proteins: an anti-fluorescein scFv fused via a transmembrane domain to a 41bb-zeta intracellular domain; and either an anti-CD19 scFv fused to a FRalpha domain in turn fused to a transmembrane stalk domain or an anti-CD19 scFv fused to a FRalpha domain secreted in the cell and lacking a stalk. A fluorescein-folate (“FITC-folate”) is used to gate binding of the two proteins.

FIG. 14 is a graph depicting IFNγ and IL-2 cytokine production in response to an increasing concentration of gating adaptor FITC-folate (EC17) in anti-FITC CAR/anti-CD19 FRalpha-stalk transduced T-cells or non-transduced control T-cell co-cultured for 12 hours with control (K562) or target Raji cells.

FIGS. 15A-15C are flow cytometry staining plots depicting surface expression of GATR constructs in transduced T-cells. FIG. 15A depicts a flow cytometry staining plot of untransduced cells. FIG. 15B depicts a flow cytometry staining plot of cells transduced with GATR FITC-CAR CD19-FRalpha. FIG. 15C depicts a flow cytometry staining plot of cells transduced with GATR FITC-CAR CD19-CA9.

### DETAILED DESCRIPTION OF THE INVENTION

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

As used in the description of the invention and the appended claims, the singular forms “a”, “an”, and “the” are intended to include the plural forms as well, unless the context clearly indicates otherwise.

“Subject” as used herein includes mammal, such as primate, mouse, rat, dog, cat, cow, horse, goat, camel, sheep or a pig, preferably a human.

“Treat,” “treating” or “treatment” as used herein also refers to any type of action or administration that imparts a benefit to a subject that has a disease or disorder, including improvement in the condition of the patient (e.g., reduction or amelioration of one or more symptoms), healing, etc.

The active compounds described herein may be formulated for administration in a pharmaceutical carrier in accordance with known techniques. See, e.g., Remington, The Science and Practice of Pharmacy (21st Ed. 2005). In the manufacture of a pharmaceutical formulation, the active compound is typically admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.01% or 0.5% to 95% or 99% by weight of the active compound. One or more active compounds may be incorporated in the formulations disclosed herein, which may be prepared by any of the well-known techniques of pharmacy comprising admixing the components, optionally including one or more accessory ingredients.

Furthermore, a “pharmaceutically acceptable” component such as a sugar, carrier, excipient or diluent of a composition according to the present disclosure is a component that (i) is compatible with the other ingredients of the composition in that it can be combined with the compositions of the present disclosure without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as saline solutions, water, emulsions such as oil/water emulsion, microemulsions and various types of wetting agents.

Also as used herein, “and/or” refers to and encompasses any and all possible combinations of one or more of the associated listed items, as well as the lack of combinations when interpreted in the alternative (or).

Unless the context indicates otherwise, it is specifically intended that the various features described herein can be used in any combination. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed.

It will also be understood that, as used herein, the terms example, exemplary, and grammatical variations thereof are intended to refer to non-limiting examples and/or variant embodiments discussed herein, and are not intended to indicate preference for one or more embodiments discussed herein compared to one or more other embodiments.

All publications, patent applications, patents and other references cited herein are incorporated by reference in their entireties for the teachings relevant to the sentence and/or paragraph in which the reference is presented.

Unless the context indicates otherwise, it is specifically intended that the various features described herein can be used in any combination.

Moreover, the present disclosure also contemplates that in some embodiments, any feature or combination of features set forth herein can be excluded or omitted.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence. The nucleic acid may produce a polypeptide which comprises one or more sequences encoding a mitogenic transduction enhancer and/or one or more sequences encoding a cytokine-based transduction enhancer. The cleavage site may be self-cleaving, such that when the polypeptide is produced, it is immediately cleaved into the receptor component and the signaling component without the need for any external cleavage activity.

As used herein the term “sequence identity”, or “identity” in relation to polynucleotides or polypeptide sequences, refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite or Clustal Omega sequence analysis programs. Unless noted otherwise, the term “sequence identity” in the claims refers to sequence identity as calculated by BLAST version 2.11.0 using default parameters. And, unless noted otherwise, the alignment is an alignment of all or a portion of the polynucleotide or polypeptide sequences of interest across the full length of the reference sequence.

### Overview

The present disclosure provides a cell biology system comprising (a) a gating adaptor; (b) a targeting adaptor, or a vector encoding the targeting adaptor; and (c) an engineered immune cell comprising a chimeric receptor, or a vector encoding the chimeric receptor. The targeting adaptor comprises a first ligand-binding domain (tLBD-1) specific for a cell-surface antigen and a second ligand-binding domain (tLBD-2) specific for the gating adaptor. The chimeric receptor comprises an extracellular ligand-binding domain (rLBD) specific for the gating adaptor, a transmembrane domain, and an intracellular actuator domain.

As the system is “gated” by presence or absence of the gating adaptor, this system is termed Gated Adaptor Targeting Receptor (GATR) system. The gating adaptor may be a small molecule. General administration, dosing, and pharmacokinetics are favorable for temporal control of the GATR system. However, in variations of the system, the gating adaptor may comprise a polypeptide and/or polynucleotide components, as various bifunctional molecules may be used to control association of the targeting adaptor to the chimeric receptor.

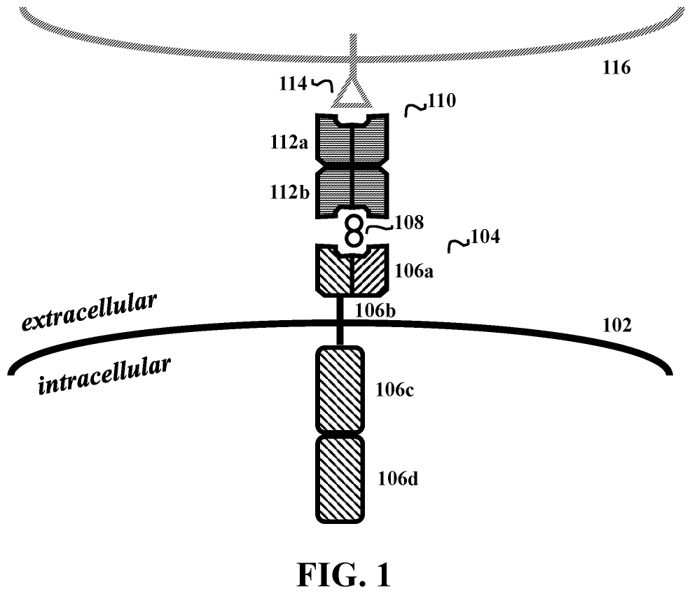

An embodiment of the GATR system is shown in FIG. 1. The cellular membrane of an engineered cell 102 is spanned from extracellular to intracellular by a chimeric receptor comprising an extracellular ligand-binding domain (rLBD) specific for the gating adaptor 106a, a transmembrane domain 106b, and two intracellular actuator domains (106c and 106d). The gating adaptor 108 binds both the chimeric receptor 104 and the targeting adaptor 110, which comprises a first ligand-binding domain (tLBD-1) 112a specific for a cell-surface antigen 114 and a second ligand-binding domain (tLBD-2) 112b specific for the gating adaptor 108. This targets the engineered cell 102 to a target cell 116. The chimeric receptor is expressed by the engineered cell from a polynucleotide introduced into the engineered cell, transiently or stably, in vivo or ex vivo, by methods known in the art (viral or non-viral vectors). The targeting adaptor may be expressed by the engineered cell, expressed by other cells in the body of the subject, or administered to the subject as a recombinant protein.

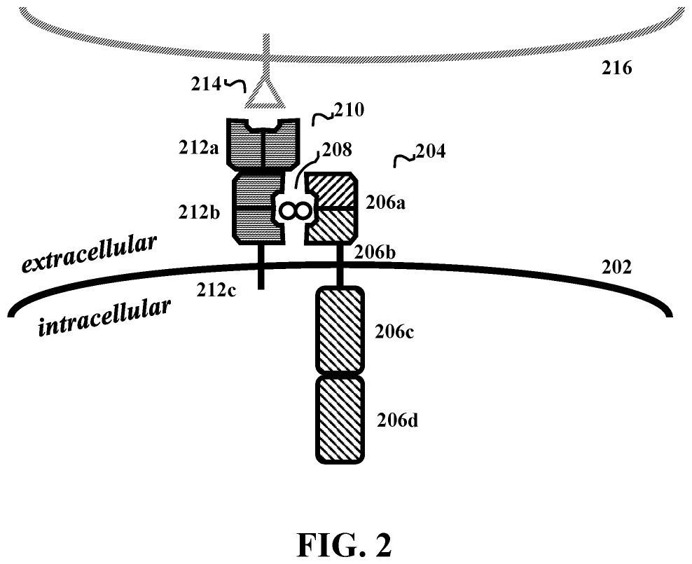

Another embodiment of the GATR system is shown in FIG. 2. The cellular membrane of an engineered cell 202 is spanned from extracellular to intracellular by a chimeric receptor comprising an extracellular ligand-binding domain (rLBD) specific for the gating adaptor 206a, a transmembrane domain 206b, and two intracellular actuator domains (206c and 206d). The gating adaptor 208 binds both the chimeric receptor 204 and the targeting adaptor 210, which comprises a first ligand-binding domain (tLBD-1) 212a specific for a cell-surface antigen 214 and a second ligand-binding domain (tLBD-2) 212b specific for the gating adaptor 208, and a transmembrane tether 212c that tethers the targeting adaptor to the engineered cell. This targets the engineered cell 202 to a target cell 216. The chimeric receptor is expressed by the engineered cell from a polynucleotide introduced into the engineered cell, transiently or stably, in vivo or ex vivo, by methods known in the art (viral or non-viral vectors). The targeting adaptor is also expressed by the engineered cell, from the same or a different polynucleotide, and post-translationally embedded in the cellular membrane. One advantage of this embodiment may be that tethering the targeting adaptor to the engineered cell reduces the amount of targeting adaptor required by increasing its effective concentration. Another advantage may be the increase in effective affinity of the gating adaptor, as two-dimensional diffusion of the targeting adaptor in the cellular membrane would be expected to increase the association rate of the targeting adaptor for the gating adaptor.

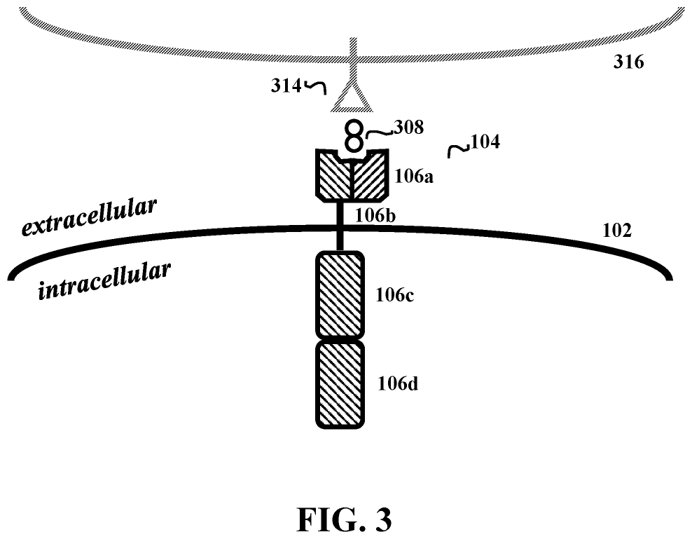

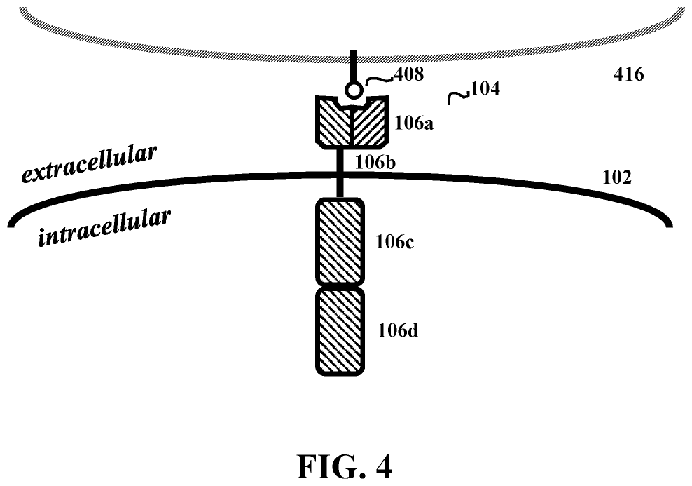

As shown in FIG. 3 and FIG. 4, some embodiments of the GATR system permit re-use of the engineered cell in other contexts. For example, FIG. 3 shows the same engineered cell line 102 expressing a chimeric receptor comprising an extracellular ligand-binding domain (rLBD) specific for the gating adaptor 106a, a transmembrane domain 106b, and two intracellular actuator domains (106c and 106d) could be used for a targeting cell 316 via a small molecule 308 recognized by a receptor 314 to a targeting cell 316.

And FIG. 4 shows the same engineered cell line 102 expressing a chimeric receptor comprising an extracellular ligand-binding domain (rLBD) specific for the gating adaptor 106a, a transmembrane domain 106b, and two intracellular actuator domains (106c and 106d) could be used for a targeting cell 416 via a membrane-binding small molecule 408 on a targeting cell 416.

The GATR systems may be distinguished from other chemically inducible dimerization (CID)-based systems in that the gating adaptor is bifunctional. For example, the Dimerizing Agent Regulated Immune-receptor Complex (DARIC) platform described in U.S. Pat. No. 10,457,731 employs a drug-induced multimerization domain to inducibly assemble a CAR or bispecific antibody (e.g., rapamycin induced association of FKBP with FRB). The GATR system uses the second ligand-binding domain (tLBD-2) of the targeting adaptor and an extracellular ligand-binding domain (rLBD) on a standalone chimeric receptor in place of the multimerization domain. One advantage of the GATR system is that a single population of engineered cells may be use to target the engineered cell directly to cells (e.g. with a small molecule conjugate that directly binds target cells) or in the dual adaptor system (with a small molecule conjugate that binds a targeting adaptor). An advantage of the present system over DARIC is that a wider variety of gating adaptors may be used—as the system does not require a drug-inducible multimerization domain (e.g. FKBP/FRB) but instead can use any two ligand binding domains that each bind different moieties of the gating adaptor.

Another distinction from DARIC of some embodiments is that the chimeric receptor may use an scFv or TCR extracellular domain as the ligand binding domain. This permits re-use of the engineered cell for direct targeting (as shown in FIG. 3 and FIG. 4) whereas the DARIC depends on having a cognate multimerization domain.

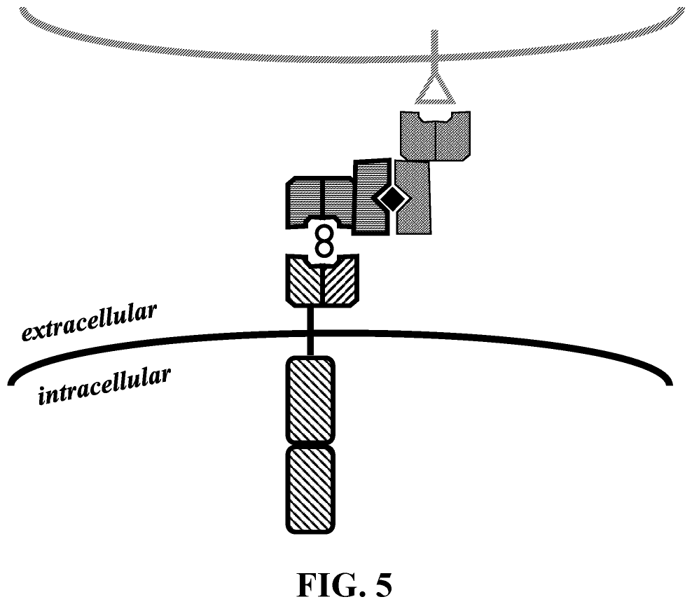

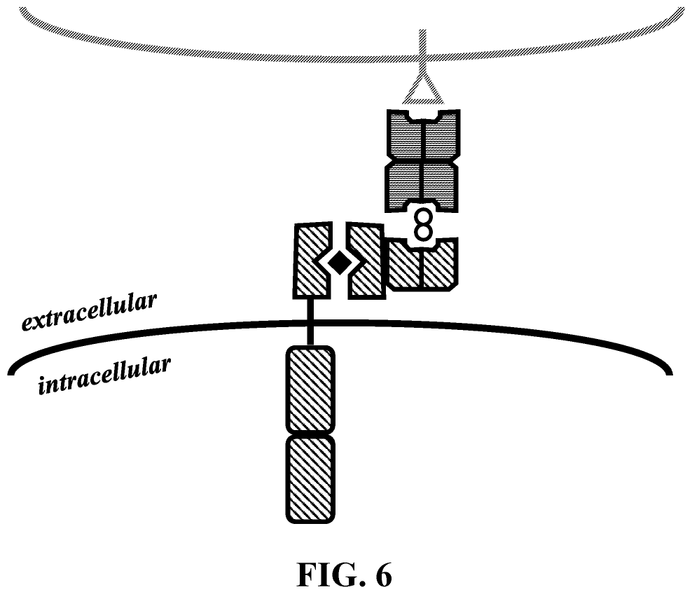

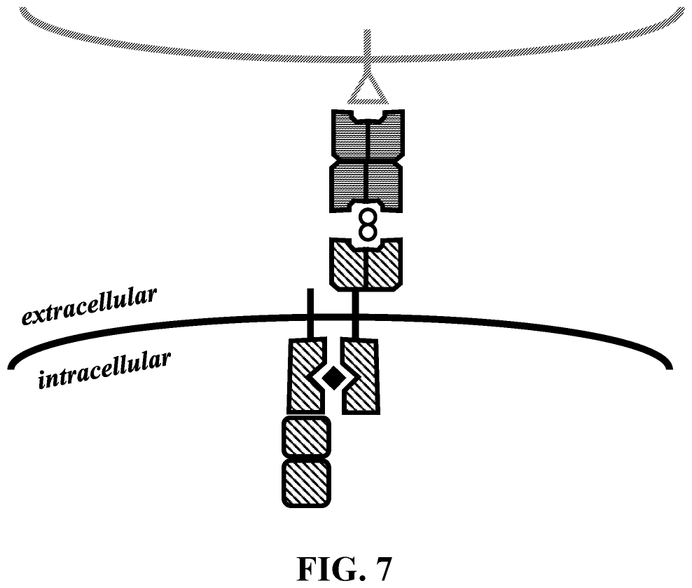

These distinctions from the DARIC system do not exclude incorporating a DARIC-like system into GATR. In some embodiments, the chimeric receptor or the targeting adaptor may include a CID-based multimerization domain pair. The paired multimerization domains may be inserted in the targeting adaptor (FIG. 5), the extracellular portion of the chimeric receptor (FIG. 6), or the intracellular portion of the chimeric receptor (FIG. 7).

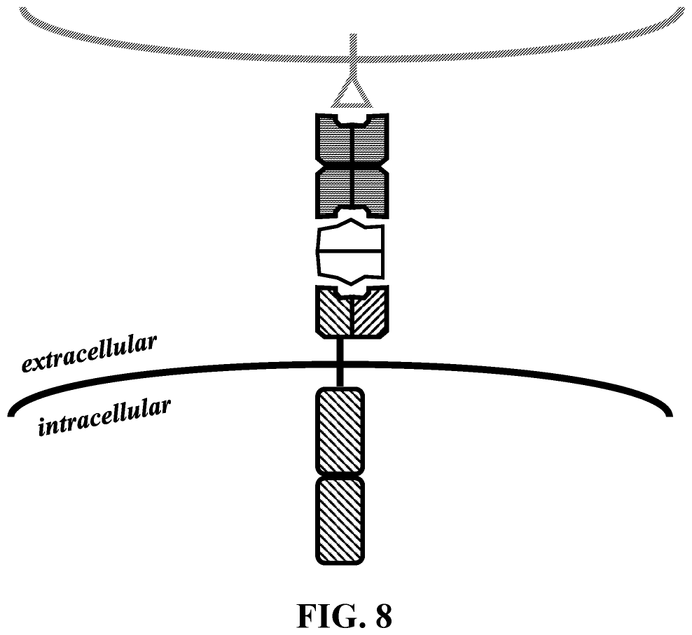

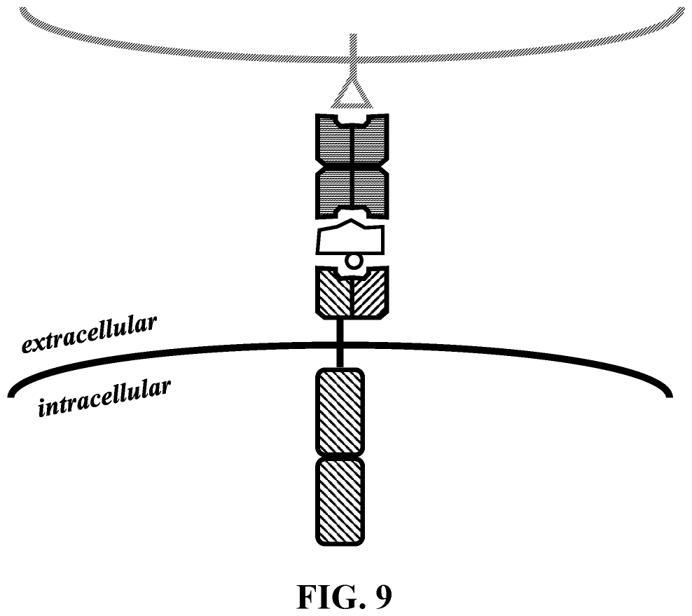

In further embodiments, the gating adaptor is a macromolecule (FIG. 8) or a small molecule conjugate of a macromolecule (FIG. 9).

### Gating Adaptor

In various embodiments of the compositions and methods of the disclosure, the system comprises a gating adaptor. The gating adaptor may comprise a first moiety and a second moiety each independently selected to be any two of small molecules for which a ligand binding domain can be generated. Thus, known small molecules useful as moieties of the gating adaptor may be used, or antibodies (or other binding domains) can be generated against novel small molecules. Illustrative small molecules useful as first or second moieties of the gating adaptor include, without limitation: rapamycin, fluorescein, fluorescein isothiocyanate (FITC), 4-[(6-methylpyrazin-2-yl) oxy] benzoic acid (aMPOB), folate, rhodamine, acetazolamide, and a CA9 ligand.

In some embodiments, the gating adaptor comprises a first moiety recognized by rLBD and a second moiety recognized by tLBD-2.

In some embodiments, the gating adaptor comprises a first moiety comprising folate, fluorescein, aMPOB, acetazolamide, a CA9 ligand, tacrolimus, rapamycin, a rapalog (a rapamycin analog), CD28 ligand, poly(his) tag, Strep-tag, FLAG-tag, VS-tag, Myc-tag, HA-tag, NE-tag, biotin, digoxigenin, dinitrophenol, or a derivative thereof.

In some embodiments, the second moiety comprises folate, acetazolamide, a CA9 ligand, fluorescein, aMPOB, tacrolimus, rapamycin, a rapalog (a rapamycin analog), CD28 ligand, poly(his) tag, Strep-tag, FLAG-tag, VS-tag, Myc-tag, HA-tag, NE-tag, biotin, digoxigenin, dinitrophenol, or a derivative thereof.

In some embodiments, the gating adaptor comprises a first moiety comprising fluorescein and a second moiety comprises folate.

In some embodiments, the gating adaptor comprises a first moiety comprising aMPOB and a second moiety comprises folate.

In some embodiments, the gating adaptor comprises a first moiety comprising fluorescein and a second moiety comprises a CA9 ligand.

In some embodiments, the gating adaptor comprises a first moiety comprising aMPOB and a second moiety comprises a CA9 ligand.

In some embodiments, the gating adaptor comprises a first moiety comprising fluorescein and a second moiety comprises acetazolamide.

In some embodiments, the gating adaptor comprises a first moiety comprising aMPOB and a second moiety comprises acetazolamide.

In some embodiments, the gating adaptor comprises a first moiety comprising fluorescein and a second moiety comprises rapamycin.

In some embodiments, the gating adaptor comprises a first moiety comprising aMPOB and a second moiety comprises rapamycin.

In some embodiments, a small molecule may be an inorganic or organic compound that is less than 1000 daltons.

In some embodiments, gating adaptor may comprise rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophernolic acid, benidipine hydrochloride, rapamine, AP23573, AP1903, or metabolites, derivatives, and/or combinations thereof.

In some embodiments, the gating adaptor comprises FK1012, tacrolimus (FK506), FKCsA, rapamycin, coumermycin, gibberellin, HaXS, TMP-HTag, or ABT-737 or functional derivatives thereof.

Illustrative IMID-class drugs useful as first or second moieties of the gating adaptor include, without limitation: thalidomide, pomalidomide, lenalidomide or related analogues.

Illustrative gating adaptors include, without limitation, a folate, 2-3-(1,3-Dicarboxy-propyl)ureido pentanedioic acid (DUPA), an NK-1R ligand, a CAIX ligand, a ligand of gamma glutamyl transpeptidase, an NKG2D ligand, or a cholecystokinin 2 receptor (CCK2R) ligand.

In some embodiments, the gating adaptor is present or provided in an amount from 0 nM to 10000 nM such as e.g., 0.05 nM, 0.1 nM, 0.5. nM, 1.0 nM, 5.0 nM, 10.0 nM, 15.0 nM, 20.0 nM, 25.0 nM, 30.0 nM, 35.0 nM, 40.0 nM, 45.0 nM, 50.0 nM, 55.0 nM, 60.0 nM, 65.0 nM, 70.0 nM, 75.0 nM, 80.0 nM, 90.0 nM, 95.0 nM, 100 nM, 200 nM, 300 nM, 400 nM, 500 nM, 600 nM, 700 nM, 800 nM, 900 nM, 1000 nM, 1500 nM, 2000 nM, 2500 nM, 3000 nM, 3500 nM, 4000 nM, 4500 nM, 5000 nM, 5500 nM, 6000 nM, 6500 nM, 7000 nM, 7500 nM, 8000 nM, 8500 nM, 9000 nM, 9500 nM, or 10000 nM, or an amount that is within a range defined by any two of the aforementioned amounts.

In some embodiments, the gating adaptor is present or provided at 1 nM.

In some embodiments, the gating adaptor is present or provided at 10 nM.

In some embodiments, the gating adaptor is present or provided at 100 nM.

In some embodiments, the gating adaptor is present or provided at 1000 nM.

In some embodiments, the gating adaptor is present or provided at 10000 nM.

With these small molecules, various gating adaptors may be generated, including without limitation a folate-fluorescein conjugate, a CA9 ligand-fluorescein conjugate, a folate-rapamycin conjugate, CA9 ligand-rapamycin conjugate, a folate-rhodamine conjugate, a acetazolamide-fluorescein conjugate or a rapamycin-fluorescein conjugate.

In some embodiments, the gating adaptor is a folate-fluorescein conjugate.

An illustrative folate-fluorescein conjugate is EC17, which has been tested clinically, e.g., in immune therapy for renal cell carcinoma. Amato et al. J. Immunotherapy 36:268-275 (2013). Thus, in some embodiments the gating adaptor is EC17. The structure of EC17 is:

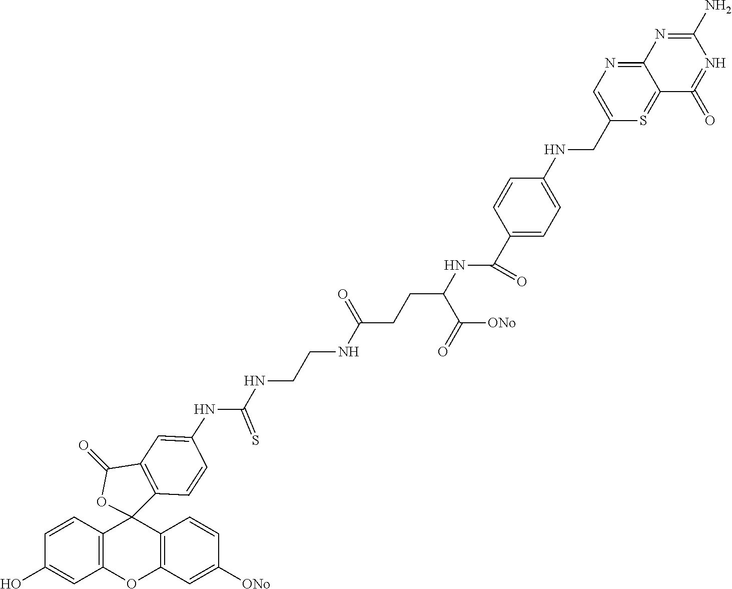

In some embodiments, the gating adaptor is a CA9 ligand-fluorescein conjugate.

In some embodiments, the gating adaptor is a rapamycin-fluorescein conjugate.

In some embodiments, the gating adaptor is an acetazolamide-fluorescein conjugate.

In some embodiments, the gating adapter may comprise a ligand of the CA9 domain with the structure:

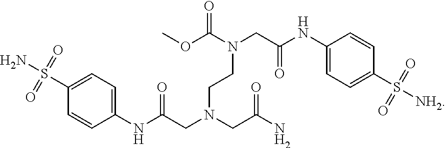 [SMILES: COC(=O)N(CCN(CC(N)=O)CC(=O)Nc1ccc(S(N)(=O)=O)cc1)CC(=O)Nc1ccc(S(N)(=O)=O)cc1]

In some embodiments, the gating adapter may comprise a ligand of the CA9 domain conjugated to fluorescein isothiocyanate with the structure:

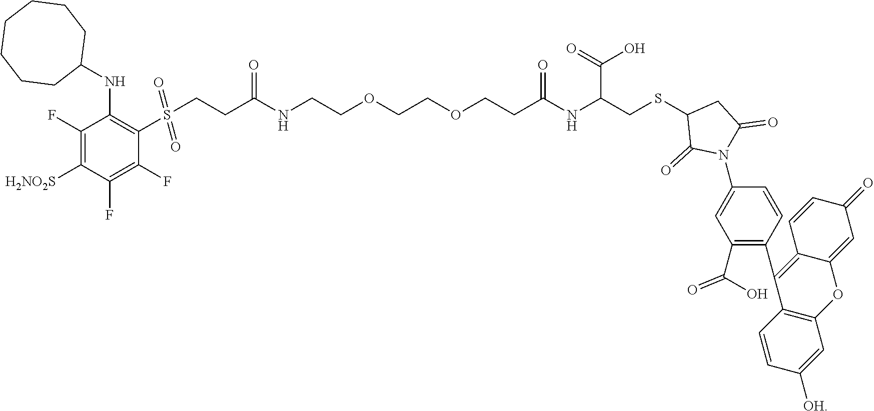 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

In some embodiments, the gating adapter may comprise a ligand of the CA9 domain conjugated to fluorescein isothiocyanate with the structure:

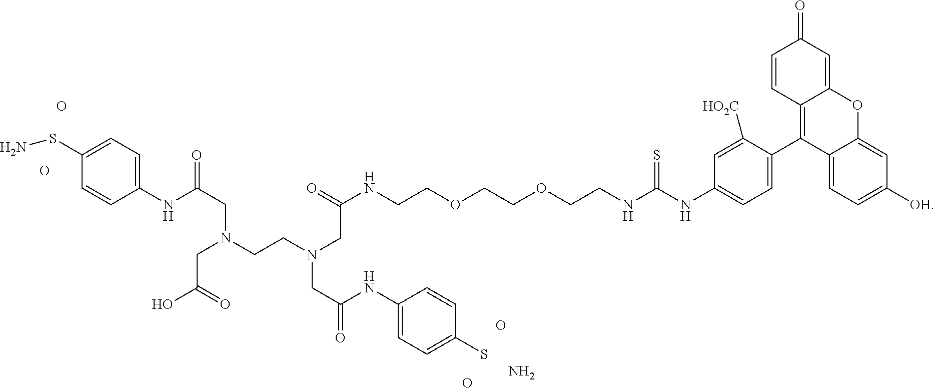

In some embodiments, the gating adapter may comprise a ligand of the CA9 domain, with the structure:

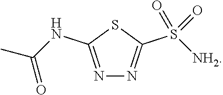

As used herein, a “gating adapter” refers to any moiety capable of being specifically recognized by a receptor, such as a chimeric receptor or the like. In some embodiments, the adaptor molecule contains more than one gating adapter, such as 2, 3, 4, 5, 6, or more gating adapters. The gating adapters may be the same or different from one another. In general the gating adapter (or gating adapters) is covalently linked to the targeting moiety directly or via a spacer. Several types of “spacers” are contemplated for use with embodiments described herein including, without limitation, a poly(carboxybetaine), peptide, polyglycidols, polyethylene, polyanhydrides, polyphosphoesters, polycaprolactone, poly(ethylene oxide), PEG spacer, a small peptide or an alkane chain. In some embodiments, the alkane spacer can comprise 2, 3, 4, 5, 6, 7, 8, 9, 10, 11, 12, 13, 14, 15, 16, 17, or 18 carbons, or any number of carbons in between a range defined by any two aforementioned values. In some embodiments, the PEG spacer comprises 1, 2, 3, 4, 5, 6, 7, 8, 9, 10, 11, 12, 13, 14, 15, 16, 17, 18, 19, 20 or 21 PEG molecules, or any amount of PEG molecules that is within a range defined by any two aforementioned values.

Various small-molecule gating adapters are known in the art. Illustrative gating adapters for use in the compositions and methods of the disclosure include those described in International Pat. Appl. Pub. No. WO2018148224, which is incorporated by reference herein in its entirety. In another aspect, the present disclosure contemplates selecting a novel gating adapter and generating an antibody specific to that gating adapter using anti-gating adapter antibody production techniques known in the art. In some embodiments, the gating adapter comprises a fluorescein. In some embodiments, the gating adapter comprises aMPOB. Recombinant human antibody E2 is an antibody capable of binding to fluorescein.

In some embodiments, the gating adapter comprises a fluorescein and the gating adapter-binding receptor comprises an anti-fluorescein antibody or antigen-binding fragment thereof, e.g., antibody E2. In some embodiments, the gating adapter comprises 2,4-dinitrophenol (DNP).

In some embodiments, the adaptor molecule comprises one or more masking moieties covalently linked to the gating adapter, thereby producing a “masked gating adapter” comprising at least one gating adapter and at least one masking moiety. A “masking moiety” is a chemical moiety that prevents or inhibits binding to the masked form of the gating adapter of ligands or receptors that are normally able to bind the unmasked form of the gating adapter. A masking moiety may include a protective group to prevent recognition of the gating adapter by blocking binding and recognition of a gating adapter-binding receptor (e.g., a chimeric receptor) that is specific for the gating adapter. When the adaptor molecule is integrated into a cell, wherein the cell exists in a tumor environment or site of reactive oxygen species, the masking moiety can be self-cleaved, thus allowing binding and recognition of the gating adapter by the chimeric receptor. In some embodiments, the targeting moiety is a lipid that is a phospholipid ether. In some embodiments, the masking moiety comprises a phenolic hydroxyl group or PEG. In some embodiments, the phenolic hydroxyl group is bound to a hydroxyl on a xanthene moiety of fluorescein. In some embodiments, the masking moiety is bound to the adaptor molecule by a cleavable moiety, which is optionally configured to be specifically cleavable in a tumor microenvironment. In some embodiments, the cleavable moiety, which is configured to be cleavable in a tumor microenvironment, is cleaved by a reactive oxygen species reaction, an acidic pH, hypoxia, or nitrosylation. In some embodiments, the phospholipid ether comprises a gating adapter and the chimeric receptor is joined to said phospholipid ether through an interaction with said gating adapter. In some embodiments, the phospholipid ether comprises a polar-head group and a carbon alkyl chain. In some embodiments, the carbon alkyl chain comprises at least 8, 9, 10, 11, 12, 13, 14, 15, 16, 17, 18, 19, 20, 21 or 22 carbons or any number that is within a range defined by any two aforementioned values. In some embodiments, the carbon alkyl chain comprises 8-22 carbons, such as 8-12, 12-14, 14-16, or 16-22 carbons. In some embodiments, the masking moiety is removed when the composition is within an acidic environment. In some embodiments, the acidic environment comprises a pH or 4, 5, 6 or 6.5 or any pH in between a range defined by any two aforementioned values. In some embodiments, the masking moiety is removed by nitrosylation.

In some embodiments, the masked gating adapter is configured to permit a chemical reaction to remove the masking moiety from the gating adapter. In some embodiments, the masked gating adapter is configured to permit reactive oxygen species to remove the masking moiety from the gating adapter. In some embodiments, the masked gating adapter comprises a hydroxyphenyl group. In some embodiments, the masking moiety comprises a 2,4-dinitrophenol (DNP) group. In some embodiments, the gating adapter comprises a fluorescein. In some embodiments, the masked gating adapter comprises a hydroxyphenyl fluorescein (HPF). In some embodiments, the masked gating adapter comprises a fluorescein-DNP.

### Targeting Adaptor

In various embodiments of the compositions of methods of the disclosure, the system comprises a targeting adaptor, or a vector encoding the targeting adaptor. The targeting adaptor comprises a first ligand-binding domain (tLBD-1) specific for a cell-surface antigen and a second ligand-binding domain (tLBD-2) specific for the gating adaptor.

First Ligand-Binding Domain (tLBD-1)

The first ligand-binding domain (tLBD-1) may be specific to a cell-surface antigen comprising ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gpl20 or a derivate, variant or fragment of these surface antigens.

In some embodiments, the tLBD-1 is specific for the cell-surface antigen CD19 or a fragment thereof.

In some embodiments, the tLBD-1 may comprise an antibody or antigen-binding fragment thereof, a single-chain variable fragment (scFv), or a T-cell receptor (TCR) or antigen-binding fragment thereof.

In some embodiments, the tLBD-1 is an antibody or an antigen-binding fragment thereof. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The term is used in the broadest sense and includes polyclonal and monoclonal antibodies, including intact antibodies and functional (antigen-binding) antibody fragments, including fragment antigen-binding (Fab) fragments, F(ab′)2 fragments, Fab′ fragments, Fv fragments, recombinant IgG (rlgG) fragments, single chain antibody fragments, including single chain variable fragments (scFv), diabodies, and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The term “antibody fragment” refers to a portion of an intact antibody and refers to the antigenic determining variable regions of an intact antibody. Examples of antibody fragments include, but are not limited to, fragment antigen-binding (Fab) fragments, F(ab′)2 fragments, Fab′ fragments, Fv fragments, recombinant IgG (rlgG) fragments, single chain antibody fragments, including single chain variable fragments (scFv), single domain antibodies (e.g., sdAb, sdFv, nanobody) fragments, diabodies, and multispecific antibodies formed from antibody fragments. In a specific embodiment, the antibody fragment is an scFv. Non-limiting examples of an antibody or binding fragment thereof include monoclonal antibodies, bispecific antibodies, Fab, Fab2, Fab3, scFv, Bis-scFv, minibody, triabody, diabody, tetrabody, VhH domain, V-NAR domain, IgNAR, and camel Ig. Additional examples of an antibody are IgG (e.g., IgG1, IgG2, IgG3, or IgG4), IgM, IgE, IgD, and IgA. Non-limiting examples of antibodies include human antibodies, humanized antibodies, or chimeric antibodies. Non-limiting examples of recombinant antibodies include antibodies that specifically bind to a tumor antigen.

In some embodiments, the tLBD-1 may comprise an anti-CD19 scFv that has at least 80% amino acid identity, at least 90% amino acid identity or at least 95% amino acid identity to:

(SEQ ID NO: 29)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVS
S.

In some embodiments, the tLBD-1 may comprise anti-CD19 scFv and may have at least 80% amino acid identity, at least 90% amino acid identity or at least 95% amino acid identity to:

(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVS
S.

In some embodiments, the tLBD-1 may comprise an anti-CD19 VL comprising at least one CDRL having at least 80% amino acid identity, at least 90% amino acid identity or at least 95% amino acid identity to an underlined nucleic acid sequence of SEQ ID NO: 52.

In some embodiments, the tLBD-1 may comprise an anti-CD19 VL comprising CDRL1, CDRL2, and CDRL3 having at least 80% amino acid identity, at least 90% amino acid identity or at least 95% amino acid identity to: QDISKYLN (SEQ ID NO: 53), LLIYHTSRLHS (SEQ ID NO: 54), and QQGNTLPY (SEQ ID NO: 55), respectively, and an anti-CD19 VH comprising CDRH1, CDRH2, and CDRH3 having at least 80% amino acid identity, at least 90% amino acid identity or at least 95% amino acid identity to: SYWMN (SEQ ID NO: 62), QIWPGDGDTNYNGKFKG (SEQ ID NO: 63), and RETTTVGRYYYAMDY (SEQ ID NO: 64), respectively.

In some embodiments, the anti-CD19 scFv is expressed with a leader sequence and has at least 80% amino acid identity, at least 90% amino acid identity or at least 95% amino acid identity to:

(SEQ ID NO: 7)
METDTLLLWVLLLWVPGSTGSDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGSTSGSGKPGSG
EGSTKGEVKLQESGPGLVAPSQSLSVTCTVSGVSL
PDYGVSWIRQPPRKGLEWLGVIWGSETTYYNSALK
SRLTIIKDNSKSQVFLKMNSLQTDDTAIYYCAKHY
YYGGSYAMDYWGQGTSVTVSS.

In some embodiments, the tLBD-1 may comprise an anti-EGFR antibody ABT-806 comprising at least one CDR having at least 80% amino acid identity, at least 90% amino acid identity or at least 95% amino acid identity to an underlined nucleic acid sequence of SEQ ID NOs: 46, 47, 48, 49, 50, 51, 56 or 57.

In some embodiments, the tLBD-1 may comprise the VL domain of anti-EGFR antibody ABT-806 and may have at least 80% amino acid identity, at least 90% amino acid identity or at least 95% amino acid identity to:

(SEQ ID NO: 46)
DILMTQSPSSMSVSLGDTVSITCHSSQDINSNIGW
LQQRPGKSFKGLIYHGTNLDDEVPSRFSGSGSGAD
YSLTISSLESEDFADYYCVQYAQFPWTFGGGTKLE
IKRA.

In some embodiments, the tLBD-1 may comprise the VH domain of anti-EGFR antibody ABT-806 and may have at least 80% amino acid identity, at least 90% amino acid identity or at least 95% amino acid identity to:

(SEQ ID NO: 48)
EVQLQESGPSLVKPSQSLSLTCTVTGYSITSDFAW
NWIRQFPGNKLEWMGYISYSGNTRYNPSLKSRISI
TRDTSKNQFFLQLNSVTIEDTATYYCVTAGRGFPY
WGQGTLVTVSS.

In some embodiments, the tLBD-1 may comprise the light chain of anti-EGFR antibody ABT-806 and may have at least 80% amino acid identity, at least 90% amino acid identity or at least 95% amino acid identity to:

(SEQ ID NO: 47)
DIQMTQSPSSMSVSVGDRVTITCHSSQDINSNIGW
LQQKPGKSFKGLIYHGTNLDDGVPSRFSGSGSGTD
YTLTISSLQPEDFATYYCVQYAQFPWTFGGGTKLE
IKRTVAAPSVFIFPP.

In some embodiments, the tLBD-1 may comprise the light chain of anti-EGFR antibody ABT-806 and may have at least 80% amino acid identity, at least 90% amino acid identity or at least 95% amino acid identity to:

(SEQ ID NO: 57)
DIQMTQSPSSMSVSVGDRVTITCHSSQDINSNIGW
LQQKPGKSFKGLIYHGTNLDDGVPSRFSGSGSGTD
YTLTISSLQPEDFATYYCVQYAQFPWTFGGGTKLE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC.

In some embodiments, the tLBD-1 may comprise the heavy chain of anti-EGFR antibody ABT-806 and may have at least 80% amino acid identity, at least 90% amino acid identity or at least 95% amino acid identity to:

(SEQ ID NO: 49)
EVQLQESGPGLVKPSQTLSLTCTVSGYSISSDFAW
NWIRQPPGKGLEWMGYISYSGNTRYQPSLKSRITI
SRDTSKNQFFLKLNSVTAADTATYYCVTAGRGFPW
GQGTLVTVSS.

In some embodiments, the tLBD-1 may comprise the heavy chain of anti-EGFR antibody ABT-806 and may have at least 80% amino acid identity, at least 90% amino acid identity or at least 95% amino acid identity to:

(SEQ ID NO: 50)
EVQLQESGPGLVKPSQTLSLTCTVSGYSISRDFAW
NWIRQPPGKGLEWMGYISYNGNTRYQPSLKSRITI
SRDTSKNQFFLKLNSVTAADTATYYCVTASRGFPW
GQGTLVTVSS.

In some embodiments, the tLBD-1 may comprise the matured anti-EGFR antibody ABT-806 scFv and may have at least 80% amino acid identity, at least 90% amino acid identity or at least 95% amino acid identity to:

(SEQ ID NO: 51)
DIQMTQSPSSMSVSVGDRVTITCHSSQDINSNIGWLQQKPGKSFKGLIYH
GTNLDDGVPSRFSGSGSGTDYTLTISSLQPEDFATYYCVQYAQFPWTFGG
GTKLEIKRSTSGSGKPGSGEGSTKGEVQLQESGPGLVKPSQTLSLTCTVS
GYSISRDFAWNWIRQPPGKGLEWMGYISYNGNTRYQPSLKSRITISRDTS
KNQFFLKLNSVTAADTATYYCVTASRGFPWGQGTLVTVSS.

In some embodiments, the tLBD-1 may comprise the heavy chain of Depatuxizumab and may have at least 80% amino acid identity, at least 90% amino acid identity or at least 95% amino acid identity to:

(SEQ ID NO: 56)
QVQLQESGPGLVKPSQTLSLTCTVSGYSISSDFAWNWIRQPPGKGLEWMG
YISYSGNTRYQPSLKSRITISRDTSKNQFFLKLNSVTAADTATYYCVTAG
RGFPYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Second Ligand-Binding Domain (tLBD-2)

The second ligand-binding domain (tLBD-2) specific for the gating adaptor may comprise an antibody or antigen-binding fragment thereof, a folate receptor domain, a folate receptor alpha (FRα) domain, a carbonic anhydrase IX (CA9) domain, DmrA (FKBP) domain, DmrC (FRB) domain, or any combination thereof.

In some embodiments, the FRα domain has at least 80% amino acid identity, at least 90% amino acid identity or at least 95% amino acid identity to:

(SEQ ID NO: 18)
GSSRTELLNVCMNAKHHKEKPGPEDKLHEQCRPWRKNACCSTNTSQEAHK
DVSYLYRFNWNHCGEMAPACKRHFIQDTCLYECSPNLGPWIQQVDQSWRK
ERVLNVPLCKEDCEQWWEDCRTSYTCKSNWHKGWNWTSGFNKCAVGAACQ
PFHFYFPTPTVLCNEIWTHSYKVSNYSRGSGRCIQMWFDPAQGNPNEEVA
RFYAAAMS.

In some embodiments, the tLBD-2 may comprise a FRα domain conjugated to a CD4 transmembrane domain and CD4 intracellular domain (CD4IC) via a short IgG4 linker. In some embodiments, said tLBD-2 has at least 80% amino acid identity, at least 90% amino acid identity or at least 95% amino acid identity to:

(SEQ ID NO: 59)
GSSRTELLNVCMNAKHHKEKPGPEDKLHEQCRPWRKNACCSTNTSQEAHK
DVSYLYRFNWNHCGEMAPACKRHFIQDTCLYECSPNLGPWIQQVDQSWRK
ERVLNVPLCKEDCEQWWEDCRTSYTCKSNWHKGWNWTSGFNKCAVGAACQ
PFHFYFPTPTVLCNEIWTHSYKVSNYSRGSGRCIQMWFDPAQGNPNEEVA
RFYAAAMSESKYGPPCPPCPGGRMALIVLGGVAGLLLFIGLGIFFCVRCR
HRRRQ

In some embodiments, the tLBD-2 may comprise a CA9 domain conjugated to a CD4 transmembrane domain and CD4IC via a short IgG4 linker. In some embodiments, said tLBD-2 has at least 80% amino acid identity, at least 90% amino acid identity or at least 95% amino acid identity to:

(SEQ ID NO: 60)
HHWRYGGDPPWPRVSPACAGRFQSPVDIRPQLAAFSPALRPLELLGFQLP
PLPELRLRNNGHSVQLTLPPGLEMALGPGREYRALQLHLHWGAAGRPGSE
HTVEGHRFPAEIHVVHLSTAFARVDEALGRPGGLAVLAAFLEEGPEENSA
YEQLLSRLEEIAEEGSETQVPGLDISALLPSDFSRYFQYEGSLTTPPCAQ
GVIWTVFNQTVMLSAKQLHTLSDTLWGPGDSRLQLNFRATQPLNGRVIEA
SFPAGVDESKYGPPCPPCPGGRMALIVLGGVAGLLLFIGLGIFFCVRCRH
RRRQ

In some embodiments, the tLBD-2 may comprise a CA9 domain and a short IgG4 linker. In some embodiments, said tLBD-2 has at least 80% amino acid identity, at least 90% amino acid identity or at least 95% amino acid identity to:

(SEQ ID NO: 61)
HHWRYGGDPPWPRVSPACAGRFQSPVDIRPQLAAFSPALRPLELLGFQLP
PLPELRLRNNGHSVQLTLPPGLEMALGPGREYRALQLHLHWGAAGRPGSE
HTVEGHRFPAEIHVVHLSTAFARVDEALGRPGGLAVLAAFLEEGPEENSA
YEQLLSRLEEIAEEGSETQVPGLDISALLPSDFSRYFQYEGSLTTPPCAQ
GVIWTVFNQTVMLSAKQLHTLSDTLWGPGDSRLQLNFRATQPLNGRVIEA
SFPAGVDESKYGPPCPPCP

In some embodiments, the CA9 domain has at least 80% amino acid identity, at least 90% amino acid identity or at least 95% amino acid identity to:

(SEQ ID NO: 19)
HHWRYGGDPPWPRVSPACAGRFQSPVDIRPQLAAFSPALRPLELLGFQLP
PLPELRLRNNGHSVQLTLPPGLEMALGPGREYRALQLHLHWGAAGRPGSE
HTVEGHRFPAEIHVVHLSTAFARVDEALGRPGGLAVLAAFLEEGPEENSA
YEQLLSRLEEIAEEGSETQVPGLDISALLPSDFSRYFQYEGSLTTPPCAQ
GVIWTVFNQTVMLSAKQLHTLSDTLWGPGDSRLQLNFRATQPLNGRVIEA
SFPAGVD

In some embodiments, the CA9 domain has at least 80% amino acid identity, at least 90% amino acid identity or at least 95% amino acid identity to:

(SEQ ID NO: 20)
HWRYGGDPPWPRVSPACAGRFQSPVDIRPQLAAFSPALRPLELSGFQLPP
LPELRLRNNGHSVQLTLPPGLEMKLGPGREYRALQLHLHWGAAGRPGSEH
TVEGHRFPAEIHVVHLSTKYARVDEALGRPGGLAVLAAFLEEGPEENSAY
EQLLSRLEEIAEEGSETQVPGLDISALLPSDFSRYFQYEGSLTTPPCAQG
VIWTVFNQTVSLSAKQLHTLSDTLWGPGDSRLQLNFRATQPLNGRVIEAS
FPAGVD.

In some embodiments, the tLBD-2 may comprise a DmrA/FKBP domain. In some embodiments, the DmrA/FKBP domain has at least 80% amino acid identity, at least 90% amino acid identity or at least 95% amino acid identity to:

(SEQ ID NO: 39)
GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE

In some embodiments, the tLBD-2 may comprise a DmrC/FRB domain. In some embodiments, the DmrC/FRB domain has at least 80% amino acid identity, at least 90% amino acid identity or at least 95% amino acid identity to:

(SEQ ID NO: 23)
GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE

In some embodiments, the tLBD-2 may comprise a cytosolic DmrC/FRB domain expressed with a 2A self-cleaving peptide. In some embodiments, the cytosolic DmrC/FRB domain has at least 80% amino acid identity, at least 90% amino acid identity or at least 95% amino acid identity to:

(SEQ ID NO: 21)
EMWHEGLEEASRLYFGERNVKGMFEVLEPLHAMMERGPQTLKETSFNQAY
GRDLMEAQEWCRKYMKSGNVKDLLQAWDLYYHVFRRISKGSGATNFSLLK
QAGDVEENPGP

### Chimeric Receptor

In various embodiments of the compositions and methods of the disclosure, the GATR system comprises a chimeric receptor, or a vector encoding the chimeric receptor. The chimeric receptor comprises an extracellular ligand-binding domain (rLBD) specific for the gating adaptor, a transmembrane domain, and an intracellular actuator domain.

The extracellular ligand-binding domain (rLBD) specific for the gating adaptor may comprise an antibody or antigen-binding fragment thereof, a single-chain variable fragment (scFv), or a T-cell receptor (TCR) or antigen-binding fragment thereof.

In some embodiments, the GATR encodes a chimeric receptor comprising an antigen binding molecule that specifically binds to a target antigen. In some embodiments, the target antigen is CD3, CD28, CD134 and CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gp120 or a derivate, variant or fragment of these surface antigens.

In some embodiments, the chimeric receptor comprises at least one polypeptide chain.

In some embodiments, the chimeric receptor comprises at least two polypeptide chains.

In some embodiments, the chimeric receptor specifically binds fluorescein.

In some embodiments, the rLBD comprises CDRL1, CDRL2, CDRL3, CDRH1, CDRH2 and CDRH3.

In some embodiments, the rLBD may comprise an anti-fluorescein antibody or fragment thereof, comprising at least one CDR having at least 80% amino acid identity, at least 90% amino acid identity or at least 95% amino acid identity to an underlined nucleic acid sequences of SEQ ID NOs: 2, 30, 31, 32 or 33.

In some embodiments, the rLBD comprises an anti-fluorescein scFv sequence selected from:

(i)
(SEQ ID NO: 2)
SVLTQPSSVSAAPGQKVTISCSGSTSNIGNNYVSWYQQHPGKAPKLMIYD
VSKRPSGVPDRFSGSKSGNSASLDISGLQSEDEADYYCAAWDDSLSEFLF
GTGTKLTVLGSTSGSGKPGSGEGSTKGQVQLVESGGNLVQPGGSLRLSCA
ASGFTFGSFSMSWVRQAPGGGLEWVAGLSARSSLTHYADSVKGRFTISRD
NAKNSVYLQMNSLRVEDTAVYYCARRSYDSSGYWGHFYSYMDVWGQGTLV
TVSS;
(ii)
(SEQ ID NO: 30)
SVLTQPSSVSAAPGQKVTISCSGSTSNIGNNYVSWYQQHPGKAPKLMIYD
VSKRPSGVPDRFSGSKSGNSASLDISGLQSEDEADYYCAAWDDSLSEFLF
GTGTKLTVLGSTSGSGKPGSGEGSTKGQVQLVESGGNLVQPGGSLRLSCA
ASGFTFGSFSMSWVRQAPGGGLEWVAGLSARSSLTHYADSVKGRFTISRD
NAKNSVYLQMNSLRVEDTAVYYCARRSYDSSGYWGHFYSYMDVWGQGTLV
TVSS;
(iii)
(SEQ ID NO: 33)
SVLTQPSSVSAAPGQKVTISCSGSTSNIGNNYVSWYQQHPGKAPKLMIYD
VSKRPSGVPDRFSGSKSGNSASLDISGLQSEDEADYYCAAWDDSLSEFLF
GTGTKLTVLGGGGGSGGGGSGGGGSQVQLVESGGNLVQPGGSLRLSCAAS
GFTFGSFSMSWVRQAPGGGLEWVAGLSARSSLTHYADSVKGRFTISRDNA
KNSVYLQMNSLRVEDTAVYYCARRSYDSSGYWGHFYSYMDVWGQGTLVTV
S.

In some embodiments, the rLBD comprises an anti-fluorescein scFv comprising CDRL1, CDRL2, and CDRL3 having at least 80% amino acid identity, at least 90% amino acid identity or at least 95% amino acid identity to: TSNIGNNYVS (SEQ ID NO: 99), LMIYDVSKRPS (SEQ ID NO: 100), and AAWDDSLSEF (SEQ ID NO: 101), respectively, and CDRH1, CDRH2, and CDRH3 having at least 80% amino acid identity, at least 90% amino acid identity or at least 95% amino acid identity to: FTFGSFSMS (SEQ ID NO: 102), WVAGLSARSSLTHY (SEQ ID NO: 103), and RRSYDSSGYWGHFYSYMDV (SEQ ID NO: 104), respectively.

In some embodiments, the rLBD comprises an anti-fluorescein scFv light chain. In some embodiments, said rLBD has at least 80% amino acid identity, at least 90% amino acid identity or at least 95% amino acid identity to:

(SEQ ID NO: 31)
SVLTQPSSVSAAPGQKVTISCSGSTSNIGNNYVSWYQQHPGKAPKLMIYD
VSKRPSGVPDRFSGSKSGNSASLDISGLQSEDEADYYCAAWDDSLSEFLF
GTGTKLTVLG

In some embodiments, the rLBD comprises an anti-fluorescein scFv heavy chain. In some embodiments, said rLBD has at least 80% amino acid identity, at least 90% amino acid identity or at least 95% amino acid identity to:

(SEQ ID NO: 32)
QVQLVESGGNLVQPGGSLRLSCAASGFTFGSFSMSWVRQAPGGGLEWVAG
LSARSSLTHYADSVKGRFTISRDNAKNSVYLQMNSLRVEDTAVYYCARRS
YDSSGYWGHFYSYMDVWGQGTLVTVSS

In some embodiments, the rLBD comprises an anti-fluorescein scFv. In some embodiments, said rLBD has at least 80% amino acid identity, at least 90% amino acid identity or at least 95% amino acid identity to:

(SEQ ID NO: 2)
SVLTQPSSVSAAPGQKVTISCSGSTSNIGNNYVSWYQQHPGKAPKLMIYD
VSKRPSGVPDRFSGSKSGNSASLDISGLQSEDEADYYCAAWDDSLSEFLF
GTGTKLTVLGSTSGSGKPGSGEGSTKGQVQLVESGGNLVQPGGSLRLSCA
ASGFTFGSFSMSWVRQAPGGGLEWVAGLSARSSLTHYADSVKGRFTISRD
NAKNSVYLQMNSLRVEDTAVYYCARRSYDSSGYWGHFYSYMDVWGQGTLV
TVSS.

In some embodiments, the chimeric receptor comprises an anti-fluorescein scFv conjugated to a CD28 transmembrane span and a 4-IBB zeta signaling tail via a short IgG4 linker. In some embodiments, said chimeric receptor has at least 80% amino acid identity, at least 90% amino acid identity or at least 95% amino acid identity to: SVLTQPSSVSAAPGQKVTISCSGSTSNIGNNYVSWYQQHPGKAPKLMIYDVSKRP

(SEQ ID NO: 58)
SGVPDRFSGSKSGNSASLDISGLQSEDEADYYCAAW
DDSLSEFLFGTGTKLTVLGSTSGSGKPGSGEGSTKG
QVQLVESGGNLVQPGGSLRLSCAASGFTFGSFSMSW
VRQAPGGGLEWVAGLSARSSLTHYADSVKGRFTISR
DNAKNSVYLQMNSLRVEDTAVYYCARRSYDSSGYWG
HFYSYMDVWGQGTLVTVSSESKYGPPCPPCPMFWVL
VVVGGVLACYSLLVTVAFIIFWVKRGRKKLLYIFKQ
PFMRPVQTTQEEDGCSCRFPEEEEGGCELRVKFSRS
ADAPAYQQGQNQLYNELNLGRREEYDVLDKRRGRDP
EMGGKPRRKNPQEGLYNELQKDKMAEAYSEIGMKGE
RRRGKGHDGLYQGLSTATKDTYDALHMQALPPR.

In some embodiments, the chimeric receptor may comprise a signal peptide comprising the amino acid sequence: MLLLVTSLLLCELPHPAFLLIP (SEQ ID NO: 1).

In some embodiments, the chimeric receptor may comprise a CD8 signal sequence comprising the amino acid sequence: MALPVTALLLPLALLLHAARP (SEQ ID NO: 22).

In some embodiments, the chimeric receptor may comprise an immunoglobulin Kappa chain signal sequence (IgKss) comprising the amino acid sequence:

(SEQ ID NO: 28)
METDTLLLWVLLLWVPGSTG.

In some embodiments, the chimeric receptor may comprise a CD28 transmembrane domain comprising the amino acid sequence:

SEQ ID NO: 4)
MFWVLVVVGGVLACYSLLVTVAFIIFWV.

In some embodiments, the chimeric receptor may comprise a CD4 transmembrane domain and CD4IC comprising the amino acid sequence:

(SEQ ID NO: 9)
GGRMALIVLGGVAGLLLFIGLGIFFCVRCRHRRRQ.

In some embodiments, the chimeric receptor may comprise a CD4 transmembrane domain comprising the amino acid sequence: MALIVLGGVAGLLLFIGLGIFF (SEQ ID NO: 40).

In some embodiments, the chimeric receptor may comprise a CD8 transmembrane domain comprising the amino acid sequence: AGTGSDIYIWAPLAGTCGVLLLSLVIT (SEQ ID NO: 24).

In some embodiments, the chimeric receptor may comprise a CD8 transmembrane domain comprising the amino acid sequence: IYIWAPLAGTCGVLLLSLVITLYC (SEQ ID NO: 45).

In some embodiments, the protein sequence may include a linker. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 96 (GGGS; SEQ ID NO: 96), SEQ ID NO: 97 (GGGSGGG; SEQ ID NO: 97) or SEQ ID NO: 98 (GGG; SEQ ID NO: 98). Embodiments also comprise a nucleic acid sequence encoding SEQ ID NOs: 96-98. In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker.

The chimeric receptor may further comprise an IgG4 linker region comprising the amino acid sequence: ESKYGPPCPPCP (SEQ ID NO: 3).

The chimeric receptor may further comprise a linker region comprising a G4S type linker comprising the amino acid sequence: ASGGGGSGGGGSGGGGS (SEQ ID NO: 8).

The chimeric receptor may further comprise a linker region comprising a G4S type linker comprising the amino acid sequence: GGGGS (SEQ ID NO: 38).

The chimeric receptor may further comprise an IgH linker comprising the amino acid sequence: ESKYGPPCPPCP (SEQ ID NO: 42).

The chimeric receptor may further comprise an IgH linker comprising the amino acid sequence: ESKYGPPCPPCPPAPEFDGG (SEQ ID NO: 43).

The chimeric receptor may further comprise a hinge region or a spacer region comprising a 2A self-cleaving peptide comprising the amino acid sequence:

(SEQ ID NO: 6)
GSGATNFSLLKQAGDVEENPGP.

The chimeric receptor may further comprise a hinge region or a spacer region comprising a P2A self-cleaving peptide comprising the amino acid sequence:

(SEQ ID NO: 27)
ATNFSLLKQAGDVEENPGP.

The chimeric receptor may further comprise a hinge region or a spacer region comprising a T2A self-cleaving peptide comprising the amino acid sequence:

(SEQ ID NO: 36)
LEGGGEGRGSLLTCGDVEENPGP.

The chimeric receptor may further comprise a hinge region comprising a CD8 stalk domain comprising the amino acid sequence:

(SEQ ID NO: 44)
TTTPAPRPPTPAPTIASQPLSLRPEACRPAAGGAVHTRGLDFACD.

The chimeric receptor may further comprise a C-region stalk domain comprising the amino acid sequence: ESKYGPPCPPCPAPEFDGG (SEQ ID NO: 34).

The chimeric receptor may further comprise a C-region stalk domain comprising the amino acid sequence:

(SEQ ID NO: 35)
PSVFLFPPKPKDTLMISRTPEVTCVVVDVSQEDPEV
QFNWYVDGVEVHNAKTKPREEQFQSTYRVVSVLTVL
HQDWLNGKEYKCKVSNKGLPSSIEKTISKAKGQPRE
PQVYTLPPSQEEMTKNQVSLTCLVKGFYPSDIAVEW
ESNGQPENNYKTTPPVLDSDGSFFLYSRLTVDKSRW
QEGNVFSCSVMHEALHNHYTQKSLSLSLGK.

In some embodiments, the chimeric receptor is a chimeric antigen receptor (CAR).

In some embodiments herein, the chimeric receptor comprises one or more intracellular actuator domains. In some embodiments, the intracellular actuator domain is derived from CD27, CD28, 4-IBB, OX40, CD30, CD40, ICOS, lymphocyte function-associated antigen-I (LFA-1), CD2, CD7, LIGHT, NKG2C, B7-H3, or a ligand that specifically binds with CD83, or a portion thereof.

In some embodiments, the intracellular actuator domain comprises a domain derived from 4-IBB.

In some embodiments, the 4-IBB intracellular actuator domain comprises the sequence:

(SEQ ID NO: 5)
KRGRKKLLYIFKQPFMRPVQTTQEEDGCSCRFPEEE
EGGCELRVKFSRSADAPAYQQGQNQLYNELNLGRRE
EYDVLDKRRGRDPEMGGKPRRKNPQEGLYNELQKDK
MAEAYSEIGMKGERRRGKGHDGLYQGLSTATKDTYD
ALHMQALPPR.

In some embodiments, the 4-IBB intracellular actuator domain comprises the 4-IBB portion of the domain and comprises the sequence:

(SEQ ID NO: 25)
KRGRKKLLYIFKQPFMRPVQTTQEEDGCSCRFPEEEEGGCEL.

In some embodiments, the 4-IBB intracellular actuator domain comprises the zeta portion of the domain and comprises the sequence:

(SEQ ID NO: 26)
RVKFSRSADAPAYQQGQNQLYNELNLGRREEYDVLD
KRRGRDPEMGGKPRRKNPQEGLYNELQKDKMAEAYS
EIGMKGERRRGKGHDGLYQGLSTATKDTYDALHMQA
LPPR.

In some embodiments, the chimeric receptor may comprise a CD4IC comprising the amino acid sequence: CVRCRHRRRQ (SEQ ID NO: 41).

In some embodiments, the chimeric receptor comprises one or more co-stimulatory domains. A “co-stimulatory domain” refers to a signaling moiety that provides to T cells a signal which, in addition to the primary signal provided by for instance the CD3 zeta chain of the TCR/CD3 complex, mediates a T cell response, including, but not limited to, activation, proliferation, differentiation, cytokine secretion, and the like. A co-stimulatory domain can include all or a portion of, but is not limited to, EGFR, tEGFR, CD27, CD28, 4-IBB, OX40, CD30, CD40, ICOS, lymphocyte function-associated antigen-I (LFA-1), CD2, CD7, LIGHT, NKG2C, B7-H3, or a ligand that specifically binds with CD83. In some embodiments, the co-stimulatory domain is an intracellular signaling domain that interacts with other intracellular mediators to mediate a cell response including activation, proliferation, differentiation and cytokine secretion, and the like. In some embodiments, herein the co-stimulatory domain comprises 4-IBB and CD3ζ. In some embodiments, a T cell is provided, wherein the T cell comprises a chimeric receptor specific for the gating adaptor on the adaptor molecule. In some embodiments, the T cell further comprises an 806 CAR (anti-EGFR806) (41BB-CD3ζ CAR).

In some embodiments, the rLBD comprises a tEGFR sequence comprising at least 80% amino acid identity, at least 90% amino acid identity or at least 95% amino acid identity to:

(SEQ ID NO: 37)
RMLLLVTSLLLCELPHPAFLLIPRKVCNGIGIGEFK
DSLSINATNIKHFKNCTSISGDLHILPVAFRGDSFT
HTPPLDPQELDILKTVKEITGFLLIQAWPENRTDLH
AFENLEIIRGRTKQHGQFSLAVVSLNITSLGLRSLK
EISDGDVIISGNKNLCYANTINWKKLFGTSGQKTKI
ISNRGENSCKATGQVCHALCSPEGCWGPEPRDCVSC
RNVSRGRECVDKCNLLEGEPREFVENSECIQCHPEC
LPQAMNITCTGRGPDNCIQCAHYIDGPHCVKTCPAG
VMGENNTLVWKYADAGHVCHLCHPNCTYGCTGPGLE
GCPTNGPKIPSIATGMVGALLLLLVVALGIGLFM.

In some embodiments, the gating adaptor-binding receptor comprises a gating adaptor-specific antigen-binding fragment of an antibody. In some embodiments, the antigen-binding fragment comprises a Fab fragment or a single-chain Fv fragment (scFv). In some embodiments, the receptor that specifically binds to the gating adaptor comprises a gating adaptor-specific chimeric receptor.

In some embodiments, the gating adaptor-binding receptor is a T cell receptor (TCR) or a functional portion thereof. A “T cell receptor” or “TCR” refers to a molecule that is found on the surface of T lymphocytes or T cells that is responsible for the recognition of fragments of antigen bound to a major histocompatibility complex molecule.

In some embodiments, the gating adaptor-binding receptor is a dimerization activated receptor initiation complex (DARIC). A DARIC provides a binding component and a signaling component that are each expressed as separate fusion proteins but contain an extracellular multimerization mechanism (bridging factor) for recoupling of the two functional components on a cell surface (see U.S. Pat. Appl. No. 2016/0311901, hereby expressly incorporated by reference in its entirety). Importantly, the bridging factor in the DARIC system forms a heterodimeric receptor complex, which does not produce significant signaling on its own. The described DARIC complexes only initiate physiologically relevant signals following further co-localization with other DARIC complexes. Thus, they do not allow for the selective expansion of desired cell types without a mechanism for further multimerization of DARIC complexes (such as by e.g., contact with a tumor cell that expresses a ligand bound by a binding domain incorporated into one of the DARIC components). Thus, as used herein, in some embodiments, the binding domains incorporated into the DARIC components bind to a gating adaptor comprised by an adaptor molecule as disclosed herein.

In some embodiments, the gating adaptor-binding portion of a gating adaptor-binding receptor may comprise an antigen-binding portion of an antibody or an antigen-binding antibody derivative. An antigen-binding portion or derivative of an antibody may be a Fab, Fab′, F(ab′)2, Fd, Fv, scFv, a diabody, a linear antibody, a single-chain antibody, a minibody, or the like. In some embodiments, the gating adaptor-binding portion of a gating adaptor-binding receptor may comprise a DARPin or centyrin.

The gating adaptor-binding receptor may bind to a molecule associated with a disease or disorder. As used herein, the molecule may be a gating adaptor comprised by an adaptor molecule. In some embodiments, the gating adaptor to which the gating adaptor-binding receptors bind or interact can be presented on a substrate, such as a membrane, bead, or support (e.g., a well) or a binding agent, such as a lipid (e.g., PLE), gating adaptor, ligand, or antibody, or binding fragment thereof. In some embodiments, the adaptor molecule is a binding agent that has specificity for an antigen present on a cancer cell. In some embodiments, the adaptor molecule is a binding agent that has specificity for a pathogen, such as a virus or bacterium. By one approach, the substrate or adaptor molecule comprising the desired gating adaptor is contacted with a plurality of cells comprising a gating adaptor-binding receptor specific for said gating adaptor and the level or amount of binding of the cells comprising the gating adaptor-binding receptor to the gating adaptor present on the substrate or binding agent is determined. Such an evaluation of binding may include staining for cells bound to adaptor molecules or evaluation of fluorescence or loss of fluorescence. Again, modifications to the gating adaptor-binding receptor structure, such as varying spacer lengths, can be evaluated in this manner. In some approaches, a target cell is also provided such that the method comprises contacting a cell, such as a T cell, which comprises a gating adaptor-binding receptor that is specific for an adaptor molecule comprising a target moiety and a gating adaptor, in the presence of a target cell, such as a cancer cell or bacterial cell, or a target virus and evaluating the binding of the cell comprising the gating adaptor-binding receptor to the adaptor molecule and/or evaluating the binding of the cell comprising the gating adaptor-binding receptor to the target cell or target virus. The variation of the different elements of the gating adaptor-binding receptor can, for example, lead to stronger binding affinity for a specific epitope or antigen.

In some embodiments described herein, the gating adaptor-binding receptor is specific for a lipid or peptide that targets a tumor or cancer cell, wherein the lipid or peptide comprises a gating adaptor and the gating adaptor-binding receptor can specifically bind to said lipid through an interaction with said gating adaptor. In some embodiments, the lipid is a phospholipid ether. In some embodiments described herein, the gating adaptor-binding receptor is specific for a phospholipid ether, wherein the phospholipid ether comprises a gating adaptor and the gating adaptor-binding receptor specifically binds to said phospholipid ether through an interaction with said gating adaptor.

In some embodiments, the gating adaptor-binding receptor is specific for a gating adaptor affixed to an antibody or binding fragment thereof, wherein the gating adaptor-binding receptor specifically binds to said antibody or binding fragment thereof through an interaction with said gating adaptor. Exemplary gating adaptors which can be conjugated to said antibody or binding fragment thereof include a poly(his) tag, Strep-tag, FLAG-tag, VS-tag, Myc-tag, HA-tag, NE-tag, biotin, digoxigenin, dinitrophenol, green fluorescent protein (GFP), yellow fluorescent protein, orange fluorescent protein, red fluorescent protein, far red fluorescent protein, or fluorescein (e.g., fluorescein isothiocyanate (FITC)). In some embodiments, the antibody or binding fragment thereof is specific for an antigen or ligand present on a cancer cell or a pathogen (e.g., viral or bacterial pathogen). In some embodiments, the antibody or binding fragment thereof is specific for an antigen or ligand present on a tumor cell, a virus, preferably a chronic virus (e.g., a hepatitis virus, such as HBV or HCV, or HIV), or a bacterial cell.

In some embodiments, the gating adaptor-binding receptor nucleic acid comprises a polynucleotide coding for a transmembrane domain. The transmembrane domain provides for anchoring of the chimeric receptor in the membrane.

In some embodiments, a complex is provided, wherein the complex comprises a gating adaptor-binding receptor joined to a lipid wherein the lipid comprises a gating adaptor and the gating adaptor-binding receptor is joined to said lipid through an interaction with said gating adaptor.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a FRα-CD19 scFv fusion, wherein the anti-CD19 scFv is membrane tethered via a transmembrane domain.

In some embodiments, the anti-CD19-FRalpha fusion subunit is a membrane-bound and contains a stalk transmembrane domain. In some embodiments, the anti-CD19-FRalpha fusion subunit is secreted and lacks the stalk transmembrane domain.

In some embodiments, the vector comprising the polypeptide encoding the GATR system comprises at least 80% amino acid identity, at least 90% amino acid identity or at least 95% amino acid identity to:

(SEQ ID NO: 10)
MLLLVTSLLLCELPHPAFLLIPSVLTQPSSVSAAPG
QKVTISCSGSTSNIGNNYVSWYQQHPGKAPKLMIYD
VSKRPSGVPDRFSGSKSGNSASLDISGLQSEDEADY
YCAAWDDSLSEFLFGTGTKLTVLGSTSGSGKPGSGE
GSTKGQVQLVESGGNLVQPGGSLRLSCAASGFTFGS
FSMSWVRQAPGGGLEWVAGLSARSSLTHYADSVKGR
FTISRDNAKNSVYLQMNSLRVEDTAVYYCARRSYDS
SGYWGHFYSYMDVWGQGTLVTVSSESKYGPPCPPCP
MFWVLVVVGGVLACYSLLVTVAFIIFWVKRGRKKLL
YIFKQPFMRPVQTTQEEDGCSCRFPEEEEGGCELRV
KFSRSADAPAYQQGQNQLYNELNLGRREEYDVLDKR
RGRDPEMGGKPRRKNPQEGLYNELQKDKMAEAYSEI
GMKGERRRGKGHDGLYQGLSTATKDTYDALHMQALP
PRGSGATNFSLLKQAGDVEENPGPMETDTLLLWVLL
LWVPGSTGSDIQMTQTTSSLSASLGDRVTISCRASQ
DISKYLNWYQQKPDGTVKLLIYHTSRLHSGVPSRFS
GSGSGTDYSLTISNLEQEDIATYFCQQGNTLPYTFG
GGTKLEITGSTSGSGKPGSGEGSTKGEVKLQESGPG
LVAPSQSLSVTCTVSGVSLPDYGVSWIRQPPRKGLE
WLGVIWGSETTYYNSALKSRLTIIKDNSKSQVFLKM
NSLQTDDTAIYYCAKHYYYGGSYAMDYWGQGTSVTV
SSASGGGGSGGGGSGGGGSGSSRTELLNVCMNAKHH
KEKPGPEDKLHEQCRPWRKNACCSTNTSQEAHKDVS
YLYRFNWNHCGEMAPACKRHFIQDTCLYECSPNLGP
WIQQVDQSWRKERVLNVPLCKEDCEQWWEDCRTSYT
CKSNWHKGWNWTSGFNKCAVGAACQPFHFYFPTPTV
LCNEIWTHSYKVSNYSRGSGRCIQMWFDPAQGNPNE
EVARFYAAAMSESKYGPPCPPCPGGRMALIVLGGVA
GLLLFIGLGIFFCVRCRHRRRQ.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a soluble FRα-CD19 scFv fusion.

In some embodiments, the vector comprising the polypeptide encoding the GATR system comprises at least 80% amino acid identity, at least 90% amino acid identity or at least 95% amino acid identity to:

(SEQ ID NO: 11)
MLLLVTSLLLCELPHPAFLLIPSVLTQPSSVSAAPG
QKVTISCSGSTSNIGNNYVSWYQQHPGKAPKLMIYD
VSKRPSGVPDRFSGSKSGNSASLDISGLQSEDEADY
YCAAWDDSLSEFLFGTGTKLTVLGSTSGSGKPGSGE
GSTKGQVQLVESGGNLVQPGGSLRLSCAASGFTFGS
FSMSWVRQAPGGGLEWVAGLSARSSLTHYADSVKGR
FTISRDNAKNSVYLQMNSLRVEDTAVYYCARRSYDS
SGYWGHFYSYMDVWGQGTLVTVSSESKYGPPCPPCP
MFWVLVVVGGVLACYSLLVTVAFIIFWVKRGRKKLL
YIFKQPFMRPVQTTQEEDGCSCRFPEEEEGGCELRV
KFSRSADAPAYQQGQNQLYNELNLGRREEYDVLDKR
RGRDPEMGGKPRRKNPQEGLYNELQKDKMAEAYSEI
GMKGERRRGKGHDGLYQGLSTATKDTYDALHMQALP
PRGSGATNFSLLKQAGDVEENPGPMETDTLLLWVLL
LWVPGSTGSDIQMTQTTSSLSASLGDRVTISCRASQ
DISKYLNWYQQKPDGTVKLLIYHTSRLHSGVPSRFS
GSGSGTDYSLTISNLEQEDIATYFCQQGNTLPYTFG
GGTKLEITGSTSGSGKPGSGEGSTKGEVKLQESGPG
LVAPSQSLSVTCTVSGVSLPDYGVSWIRQPPRKGLE
WLGVIWGSETTYYNSALKSRLTIIKDNSKSQVFLKM
NSLQTDDTAIYYCAKHYYYGGSYAMDYWGQGTSVTV
SSASGGGGSGGGGSGGGGSGSSRTELLNVCMNAKHH
KEKPGPEDKLHEQCRPWRKNACCSTNTSQEAHKDVS
YLYRFNWNHCGEMAPACKRHFIQDTCLYECSPNLGP
WIQQVDQSWRKERVLNVPLCKEDCEQWWEDCRTSYT
CKSNWHKGWNWTSGFNKCAVGAACQPFHFYFPTPTV
LCNEIWTHSYKVSNYSRGSGRCIQMWFDPAQGNPNE
EVARFYAAAMS.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a Ca9 Domain-CD19 scFv fusion, wherein the anti-CD19 scFv is membrane tethered via a transmembrane domain.

In some embodiments, the vector comprising the polypeptide encoding the GATR system comprises at least 80% amino acid identity, at least 90% amino acid identity or at least 95% amino acid identity to:

(SEQ ID NO: 12)
MLLLVTSLLLCELPHPAFLLIPSVLTQPSSVSAAPGQKVTISCSGSTSNI
GNNYVSWYQQHPGKAPKLMIYDVSKRPSGVPDRFSGSKSGNSASLDISGL
QSEDEADYYCAAWDDSLSEFLFGTGTKLTVLGSTSGSGKPGSGEGSTKGQ
VQLVESGGNLVQPGGSLRLSCAASGFTFGSFSMSWVRQAPGGGLEWVAGL
SARSSLTHYADSVKGRFTISRDNAKNSVYLQMNSLRVEDTAVYYCARRSY
DSSGYWGHFYSYMDVWGQGTLVTVSSESKYGPPCPPCPMFWVLVVVGGVL
ACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRGSGATNFSLLKQAGDVEENPGPMETDTLLL
WVLLLWVPGSTGSDIQMTQTTSSLSASLGDRVTISCRASQDISKYLNWYQ
QKPDGTVKLLIYHTSRLHSGVPSRFSGSGSGTDYSLTISNLEQEDIATYF
CQQGNTLPYTFGGGTKLEITGSTSGSGKPGSGEGSTKGEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSASGGGGSGGGGSGGGGSHHWRYGGDPPWPRVSPACAGRFQSP
VDIRPQLAAFSPALRPLELLGFQLPPLPELRLRNNGHSVQLTLPPGLEMA
LGPGREYRALQLHLHWGAAGRPGSEHTVEGHRFPAEIHVVHLSTAFARVD
EALGRPGGLAVLAAFLEEGPEENSAYEQLLSRLEEIAEEGSETQVPGLDI
SALLPSDFSRYFQYEGSLTTPPCAQGVIWTVFNQTVMLSAKQLHTLSDTL
WGPGDSRLQLNFRATQPLNGRVIEASFPAGVDESKYGPPCPPCPGGRMAL
IVLGGVAGLLLFIGLGIFFCVRCRHRRRQ.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a soluble Ca9 Domain-CD19 scFv fusion.

In some embodiments, the vector comprising the polypeptide encoding the GATR system comprises at least 80% amino acid identity, at least 90% amino acid identity or at least 95% amino acid identity to:

(SEQ ID NO: 13)
MLLLVTSLLLCELPHPAFLLIPSVLTQPSSVSAAPGQKVTISCSGSTSNI
GNNYVSWYQQHPGKAPKLMIYDVSKRPSGVPDRFSGSKSGNSASLDISGL
QSEDEADYYCAAWDDSLSEFLFGTGTKLTVLGSTSGSGKPGSGEGSTKGQ
VQLVESGGNLVQPGGSLRLSCAASGFTFGSFSMSWVRQAPGGGLEWVAGL
SARSSLTHYADSVKGRFTISRDNAKNSVYLQMNSLRVEDTAVYYCARRSY
DSSGYWGHFYSYMDVWGQGTLVTVSSESKYGPPCPPCPMFWVLVVVGGVL
ACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRGSGATNFSLLKQAGDVEENPGPMETDTLLL
WVLLLWVPGSTGSDIQMTQTTSSLSASLGDRVTISCRASQDISKYLNWYQ
QKPDGTVKLLIYHTSRLHSGVPSRFSGSGSGTDYSLTISNLEQEDIATYF
CQQGNTLPYTFGGGTKLEITGSTSGSGKPGSGEGSTKGEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSASGGGGSGGGGSGGGGSHHWRYGGDPPWPRVSPACAGRFQSP
VDIRPQLAAFSPALRPLELLGFQLPPLPELRLRNNGHSVQLTLPPGLEMA
LGPGREYRALQLHLHWGAAGRPGSEHTVEGHRFPAEIHVVHLSTAFARVD
EALGRPGGLAVLAAFLEEGPEENSAYEQLLSRLEEIAEEGSETQVPGLDI
SALLPSDFSRYFQYEGSLTTPPCAQGVIWTVFNQTVMLSAKQLHTLSDTL
WGPGDSRLQLNFRATQPLNGRVIEASFPAGVDESKYGPPCPPCP.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a mutated Ca9 Domain-CD19 scFv fusion, wherein the anti-CD19 scFv is membrane tethered via a transmembrane domain.

In some embodiments, the vector comprising the polypeptide encoding the GATR system comprises at least 80% amino acid identity, at least 90% amino acid identity or at least 95% amino acid identity to:

(SEQ ID NO: 14)
MLLLVTSLLLCELPHPAFLLIPSVLTQPSSVSAAPGQKVTISCSGSTSNI
GNNYVSWYQQHPGKAPKLMIYDVSKRPSGVPDRFSGSKSGNSASLDISGL
QSEDEADYYCAAWDDSLSEFLFGTGTKLTVLGSTSGSGKPGSGEGSTKGQ
VQLVESGGNLVQPGGSLRLSCAASGFTFGSFSMSWVRQAPGGGLEWVAGL
SARSSLTHYADSVKGRFTISRDNAKNSVYLQMNSLRVEDTAVYYCARRSY
DSSGYWGHFYSYMDVWGQGTLVTVSSESKYGPPCPPCPMFWVLVVVGGVL
ACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRGSGATNFSLLKQAGDVEENPGPMETDTLLL
WVLLLWVPGSTGSDIQMTQTTSSLSASLGDRVTISCRASQDISKYLNWYQ
QKPDGTVKLLIYHTSRLHSGVPSRFSGSGSGTDYSLTISNLEQEDIATYF
CQQGNTLPYTFGGGTKLEITGSTSGSGKPGSGEGSTKGEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSASGGGGSGGGGSGGGGSHWRYGGDPPWPRVSPACAGRFQSPV
DIRPQLAAFSPALRPLELSGFQLPPLPELRLRNNGHSVQLTLPPGLEMKL
GPGREYRALQLHLHWGAAGRPGSEHTVEGHRFPAEIHVVHLSTKYARVDE
ALGRPGGLAVLAAFLEEGPEENSAYEQLLSRLEEIAEEGSETQVPGLDIS
ALLPSDFSRYFQYEGSLTTPPCAQGVIWTVFNQTVSLSAKQLHTLSDTLW
GPGDSRLQLNFRATQPLNGRVIEASFPAGVDESKYGPPCPPCPGGRMALI
VLGGVAGLLLFIGLGIFFCVRCRHRRRQ.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a soluble mutated Ca9 Domain-CD19 scFv fusion.

In some embodiments, the vector comprising the polypeptide encoding the GATR system comprises at least 80% amino acid identity, at least 90% amino acid identity or at least 95% amino acid identity to:

(SEQ ID NO: 15)
MLLLVTSLLLCELPHPAFLLIPSVLTQPSSVSAAPGQKVTISCSGSTSNI
GNNYVSWYQQHPGKAPKLMIYDVSKRPSGVPDRFSGSKSGNSASLDISGL
QSEDEADYYCAAWDDSLSEFLFGTGTKLTVLGSTSGSGKPGSGEGSTKGQ
VQLVESGGNLVQPGGSLRLSCAASGFTFGSFSMSWVRQAPGGGLEWVAGL
SARSSLTHYADSVKGRFTISRDNAKNSVYLQMNSLRVEDTAVYYCARRSY
DSSGYWGHFYSYMDVWGQGTLVTVSSESKYGPPCPPCPMFWVLVVVGGVL
ACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRGSGATNFSLLKQAGDVEENPGPMETDTLLL
WVLLLWVPGSTGSDIQMTQTTSSLSASLGDRVTISCRASQDISKYLNWYQ
QKPDGTVKLLIYHTSRLHSGVPSRFSGSGSGTDYSLTISNLEQEDIATYF
CQQGNTLPYTFGGGTKLEITGSTSGSGKPGSGEGSTKGEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSASGGGGSGGGGSGGGGSHWRYGGDPPWPRVSPACAGRFQSPV
DIRPQLAAFSPALRPLELSGFQLPPLPELRLRNNGHSVQLTLPPGLEMKL
GPGREYRALQLHLHWGAAGRPGSEHTVEGHRFPAEIHVVHLSTKYARVDE
ALGRPGGLAVLAAFLEEGPEENSAYEQLLSRLEEIAEEGSETQVPGLDIS
ALLPSDFSRYFQYEGSLTTPPCAQGVIWTVFNQTVSLSAKQLHTLSDTLW
GPGDSRLQLNFRATQPLNGRVIEASFPAGVDESKYGPPCPPCP.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a DmrA-CD19 scFv fusion, wherein the anti-CD19 scFv is membrane tethered via a transmembrane domain.

In some embodiments, the vector comprising the polypeptide encoding the GATR system comprises at least 80% amino acid identity, at least 90% amino acid identity or at least 95% amino acid identity to:

(SEQ ID NO: 16)
MLLLVTSLLLCELPHPAFLLIPSVLTQPSSVSAAPGQKVTISCSGSTSNI
GNNYVSWYQQHPGKAPKLMIYDVSKRPSGVPDRFSGSKSGNSASLDISGL
QSEDEADYYCAAWDDSLSEFLFGTGTKLTVLGSTSGSGKPGSGEGSTKGQ
VQLVESGGNLVQPGGSLRLSCAASGFTFGSFSMSWVRQAPGGGLEWVAGL
SARSSLTHYADSVKGRFTISRDNAKNSVYLQMNSLRVEDTAVYYCARRSY
DSSGYWGHFYSYMDVWGQGTLVTVSSESKYGPPCPPCPMFWVLVVVGGVL
ACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRGSGATNFSLLKQAGDVEENPGPMETDTLLL
WVLLLWVPGSTGSDIQMTQTTSSLSASLGDRVTISCRASQDISKYLNWYQ
QKPDGTVKLLIYHTSRLHSGVPSRFSGSGSGTDYSLTISNLEQEDIATYF
CQQGNTLPYTFGGGTKLEITGSTSGSGKPGSGEGSTKGEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSASGGGGSGVQVETISPGDGRTFPKRGQTCVVHYTGMLEDGKK
FDSSRDRNKPFKFMLGKQEVIRGWEEGVAQMSVGQRAKLTISPDYAYGAT
GHPGIIPPHATLVFDVELLKLEESKYGPPCPPCPGGRMALIVLGGVAGLL
LFIGLGIFFCVRCRHRRRQ.

In some embodiments of the present disclosure, the GATR system may be expressed as a single polypeptide comprising a chimeric receptor linked to a targeting adaptor. In said embodiment, the chimeric receptor may comprise an anti-fluorescein scFv fused to a transmembrane domain and a 4-1BB signaling domain. In said embodiment, the chimeric receptor may be linked by a self-cleaving peptide to a targeting adaptor comprising a soluble DmrA-CD19 scFv fusion.

In some embodiments, the vector comprising the polypeptide encoding the GATR system comprises at least 80% amino acid identity, at least 90% amino acid identity or at least 95% amino acid identity to:

(SEQ ID NO: 17)
MLLLVTSLLLCELPHPAFLLIPSVLTQPSSVSAAPGQKVTISCSGSTSNI
GNNYVSWYQQHPGKAPKLMIYDVSKRPSGVPDRFSGSKSGNSASLDISGL
QSEDEADYYCAAWDDSLSEFLFGTGTKLTVLGSTSGSGKPGSGEGSTKGQ
VQLVESGGNLVQPGGSLRLSCAASGFTFGSFSMSWVRQAPGGGLEWVAGL
SARSSLTHYADSVKGRFTISRDNAKNSVYLQMNSLRVEDTAVYYCARRSY
DSSGYWGHFYSYMDVWGQGTLVTVSSESKYGPPCPPCPMFWVLVVVGGVL
ACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRGSGATNFSLLKQAGDVEENPGPMETDTLLL
WVLLLWVPGSTGSDIQMTQTTSSLSASLGDRVTISCRASQDISKYLNWYQ
QKPDGTVKLLIYHTSRLHSGVPSRFSGSGSGTDYSLTISNLEQEDIATYF
CQQGNTLPYTFGGGTKLEITGSTSGSGKPGSGEGSTKGEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSASGGGGSGVQVETISPGDGRTFPKRGQTCVVHYTGMLEDGKK
FDSSRDRNKPFKFMLGKQEVIRGWEEGVAQMSVGQRAKLTISPDYAYGAT
GHPGIIPPHATLVFDVELLKLEESKYGPPCPPCPGGR.

### Immune Cell

In various embodiments of the compositions of methods of the disclosure, the system comprises an engineered immune cell.

The present disclosure provides a method for making an activated transgenic immune cell, which comprises the step of contacting an immune cell with a viral vector according to any of the foregoing embodiments. The immune cells may be transduced in vivo or ex vivo. In some embodiments, the viral vectors are administered to a living subject such that the immune cells are transduced in vivo without any need to isolate and manipulate host cells ex vivo. In some embodiments, immune cells are manipulated ex vivo and then returned to the subject in need thereof.

The present disclosure provides a GATR system, which comprises an engineered immune cell comprising a chimeric receptor, or a vector encoding the chimeric receptor according to any of the foregoing embodiments. The immune cells may be transduced in vivo or ex vivo. In some embodiments, a viral vector is administered to a living subject such that the immune cells are transduced in vivo without any need to isolate and manipulate host cells ex vivo. In some embodiments, immune cells are manipulated ex vivo and then returned to the subject in need thereof.

The immune cells generally are mammalian cells, and typically are human cells, more typically primary human cells, e.g., allogeneic or autologous donor cells. The cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immune systems, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, herein, the cells provided are cytotoxic T lymphocytes. A “Cytotoxic T lymphocyte” (CTL) may include but is not limited to, for example, a T lymphocyte that expresses CD8 on the surface thereof (e.g., a CD8+ T cell). In some embodiments, such cells are preferably “memory” T cells (TM cells) that are antigen-experienced. In some embodiments, the cell is a precursor T cell. In some embodiments, the precursor T cell is a hematopoietic stem cell. In some embodiments, the cell is a CD8+ T cytotoxic lymphocyte cell selected from the group consisting of naive CD8+ T cells, central memory CD8+ T cells, effector memory CD8+ T cells and bulk CD8+ T cells. In some embodiments, the cell is a CD4+ T helper lymphocyte cell that is selected from the group consisting of naive CD4+ T cells, central memory CD4+ T cells, effector memory CD4+ T cells, and bulk CD4+ T cells.

As used herein, any reference to a transgenic T cell or transduced T cell, or the use thereof, may also be applied to any of the other immune cell types disclosed herein.

The present disclosure also provides transgenic immune cells comprising one or more exogenous nucleic acid molecules. In some embodiments, the transgenic immune cells comprise polynucleotides encoding gating adaptor-binding receptors. In some embodiments, the transgenic immune cells comprise polynucleotides encoding transduction enhancers. In some embodiments, the transgenic immune cells comprise polynucleotides encoding T cell activator proteins. In some embodiments, the transgenic immune cells comprise polynucleotides encoding gating adaptor-binding receptors and polynucleotides encoding T cell activator proteins.

### Engineered Immune Cell and Pharmaceutical Compositions Thereof

The present disclosure provides an isolated cell, comprising:
(a) a polynucleotide encoding a targeting adaptor, and/or a targeting adaptor comprising a transmembrane domain and expressed on the surface of the isolated cell; and
(b) a polynucleotide encoding a chimeric receptor, and/or a chimeric receptor expressed on the surface of the isolated cell, wherein the targeting adaptor comprises a first ligand-binding domain (tLBD-1) specific for a cell-surface antigen and a second ligand-binding domain (tLBD-2) specific for the gating adaptor; and wherein the chimeric receptor comprises an extracellular ligand-binding domain (rLBD) specific for the gating adaptor, a transmembrane domain, and an intracellular actuator domain.
   Methods of Treating Subjects with the Disclosed Compositions

The present disclosure provides methods of treating a subject in need thereof with the compositions, therapeutic compositions, cells, vectors, and polynucleotides disclosed herein. In some embodiments, the disclosure provides a method of treating cancer and/or killing cancer cells in a subject, comprising administering a therapeutically effective amount of the disclosed viral particles to the subject, wherein prior to, during, or after the administering step the subject received or receives a dose of an adaptor molecule comprising a targeting moiety and a masked targeting adaptor effective to label cancer cells with a targeting adaptor. Also provided is a method of treating a tumor and/or killing tumor cells in a subject, comprising administering an effective amount of an adaptor molecule to the subject, wherein: the adaptor molecule labels tumor cells with a masked targeting adaptor; wherein the masked targeting adaptor is activated by reactive oxygen species, generating a targeting adaptor; and wherein prior to, during, or after the administering step the subject received or received the retroviral particles according to any of the foregoing embodiments.

In some embodiments, the malignancy is a solid tumor, sarcoma, carcinoma, lymphoma, multiple myeloma, Hodgkins Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T-cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitf s lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma)), monoclonal gammapathy of undetermined significance (MGUS), plasmacytomas (e.g., plasma cell dyscrasia, solitary myeloma, solitary plas acyto a, extramedullary plasmacytoma, and multiple plasmacytoma), systemic amyloid light chain amyloidosis, POEMS syndrome (also known as Crow-Fukase syndrome, Takatsuki disease, and PEP syndrome), or a combination thereof.

In some embodiments, a method disclosed herein may be use to treat cancer and/or kill cancer cells in a subject by administering a therapeutically effective amount of the lentiviral particles according to any of the foregoing embodiments, wherein prior to the administering step the subject has received a dose of adaptor molecule comprising a targeting moiety and a targeting adaptor, effective to label cancer cells with the targeting adaptor. In some embodiments, a method disclosed herein may be used to treat cancer and/or kill cancer cells by administering a system.

The present disclosure also provides a method of treating cancer and/or killing cancer cells in a subject, comprising administering the system of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, the target cell is a tumor cell. In some embodiments, the target cell is an immune cell. In some embodiments, the immune cell is a T cell or a B cell. In some embodiments, the target cell exists in a tumor microenvironment.

In some embodiments, a transduced T cell is provided to the subject 1, 2, 3, 4, 5, 6, 7, 8, 9, 10, 11, 12, 15, 20, 24, 36, 48, 60 or 72 hours after administration of an adaptor molecule composition, or any time within a range defined by any two aforementioned values. In some embodiments, the cell is provided to the subject 1, 2, 3, 4, 5, 6, 7, 8, 9, 10, 11, 12, 15, 20, 24, 36 or 48 hours before administration of the composition, or any time within a range defined by any two aforementioned values. In some embodiments, the cell is provided to the subject within seconds or minutes, such as less than an hour, of providing the composition to the subject. In some embodiments, a boost of the cell and/or the composition is provided to the subject. In some embodiments, the viral vectors are administered directly to the subject. In some embodiments, viral vectors are administered in conjunction with T cells. In some embodiments, viral vectors and T cells are separately administered. In some embodiments, T cells are activated and transduced in vivo by administered viral vectors.

In some embodiments, an additional cancer therapy is provided, such as a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

### Modes of Administration and Dosing

The disclosed viral particles, adaptor molecules, and immune cells may be administered in a number of ways depending upon whether local or systemic treatment is desired.

In the case of adoptive cell therapy, methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, intrasynovial injection or infusions; and kidney dialytic infusion techniques. In a preferred embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing immune cells or transgenic immune cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges.

In the context of administering viral particles, the amount of viral particles and time of administration of such particles will be within the purview of the skilled artisan having benefit of the present teachings. In some embodiments, the administration of therapeutically effective amounts of the disclosed compositions may be achieved by a single administration, such as for example, a single injection of sufficient numbers of viral particles to provide therapeutic benefit to the patient undergoing such treatment. In some embodiments, the subject is provided multiple, or successive administrations of the lentiviral vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, viral particles/ml given either as a single dose, or divided into two or more administrations as may be required to achieve therapy of the particular disease or disorder being treated. In some embodiments, a subject may be administered two or more different viral vector compositions, either alone, or in combination with one or more other therapeutic drugs to achieve the desired effects of a particular therapy regimen. In some embodiments, the viral vectors are administered in combination with the transgenic immune cells. In some embodiments, the viral vectors are administered in combination with immune cells that have not yet been transduced. The phrase “in combination” may comprise at the same time or at different times within a short period of time, e.g., within one week, one day, twelve hours, six hours, one hour, thirty minutes, ten minutes, five minutes, or one minute.

In the context of administering adaptor molecules, the dose will depend on the type of target cell, the targeting moiety comprised by the adaptor molecule, and the targeting adaptor molecule comprised by the adaptor molecule. Depending on the type and severity of the disease, illustrative dosages for the adaptor molecules can range from about 1 μg/kg to about 50 mg/kg or from about 5 mg/kg to about 15 mg/kg, including but not limited to 5 mg/kg, 7.5 mg/kg, 10 mg/kg or 15 mg/kg. The frequency of administration will vary depending on the type and severity of the disease. For repeated administrations over several days or longer, depending on the condition, the treatment may be sustained until the condition, e.g., cancer, is treated or the desired therapeutic effect is achieved, as measured by methods known in the art. In some embodiments, the adaptor molecules are administered one time. In some embodiments, the adaptor molecules are administered once every 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 1 year. The adaptor molecules may be administered in combination with the viral particles and/or transgenic immune cells disclosed herein. The phrase “in combination” may comprise at the same time or at different times within a short period of time, e.g., within one week, one day, twelve hours, six hours, one hour, thirty minutes, ten minutes, five minutes, or one minute.

### Methods of Using Ex Vivo Engineered Immune Cells

The present disclosure provides a method of treating a disease or disorder in a subject in need thereof, comprising administering the GATR system to a subject.

The present disclosure further provides a method of administering the GATR system to a subject, comprising administering an engineered immune cell comprising the chimeric receptor to the subject.

The engineered cells used in the methods of the present disclosure may be autologous, syngeneic or allogeneic, with the selection dependent on the disease to be treated and the means available to do so. Suitable populations of engineered cells that may be used in the methods include, but are not limited to, any immune cells with cytolytic activity, such as T cells. Illustrative sub-populations of T cells include, but are not limited to, those expressing CD3⁺ including CD3⁺CD8⁺ T cells, CD3⁺CD4+ T cells, and NKT cells. In one aspect, the T cells are H-LA-A2+ peripheral blood mononuclear cells (PBMC) but the T cells can be of any HLA background from PBMCs and utilized in an autologous, syngeneic or allogeneic system. T cells may also be isolated from any source, including from a tumor explant of the subject being treated or intratumoral T cells of the subject being treated. For the sake of convenience, the effector cells are commonly referred to herein as T cells, but it should be understood that any reference to T cells, unless otherwise indicated, is a reference to all effector cell types as defined herein.

The cells used in the GATR system of the present disclosure are cytotoxic lymphocytes selected from cytotoxic T cells (also variously known as cytotoxic T lymphocytes, CTLs, T killer cells, cytolytic T cells, CD8⁺ T cells, and killer T cells), natural killer (NK) cells, and lymphokine-activated killer (LAK) cells. Upon activation, each of these cytotoxic lymphocytes triggers the destruction of target tumor cells.

“Natural Killer” NK cells are a cytotoxic lymphocyte that represents a major component of the innate immune system. NK cells respond to tumor formation and cells infected by viruses and induce apoptosis (cell death) in infected cells.

The NK cells used in the GATR system of the present disclosure may comprise the NK cells as described in literature as well as NK cells which express one or more markers from any source.

In some embodiments, the NK cells are defined as CD3⁻CD56⁺ cells.

In some embodiments, the NK cells are defined as CD7⁺CD127⁻NiKp46⁺ T-bet⁺ Eomes⁺ cells.

In some embodiments, the NK cells are defined as CD3⁻CD56^{dim}CD16⁺ cells.

In some embodiments, the NK cells are defined as CD3⁻CD56^{bright}CD16⁻ cells.

In some embodiments, the NK cells comprise cell surface receptors that include, but are not limited to, human killer immunoglobulin-like receptors (KIRs), mouse Ly49 family receptors, CD94-NKG2 heterodimeric receptors, NKG2D, natural cytotoxicity receptors (NCRs), or any combination thereof.

In some embodiments, the T cells or natural killer (NK) cells are allogeneic donor cells.

In some embodiments, the T cells or NK cells are autologous donor cells.

In some embodiments, the method of administering the GATR system to a subject comprises administering donor derived CAR-T cells.

In some embodiments, the method of administering the GATR system to a subject comprises administering donor derived CAR-NK cells.

In some embodiments, the method of administering the GATR system to a subject comprises administering a vector encoding the chimeric receptor to a subject.

In some embodiments, the method of administering the GATR system to a subject comprises in vivo transduction of CAR-T cells or CAR-NK cells to a subject in need thereof.

In some embodiments, the method of administering the GATR system to a subject comprises administering a targeting adaptor to the subject.

In some embodiments, the method of administering the GATR system to a subject comprises administering a vector encoding the targeting adaptor to the subject.

In some embodiments, the method of administering the GATR system to a subject comprises administering a gating adaptor to the subject.

In some embodiments, the method of administering the GATR system to a subject comprises withholding the gating adaptor from the subject when a side effect of treatment is observed.

The present disclosure provides a method of generating a GATR system and/or treating a disease or disorder in a subject in need thereof, comprising:
(a) administering to a subject an engineered immune cell comprising a chimeric receptor, or a vector encoding the chimeric receptor;
(b) administering to a subject a targeting adaptor, or vector encoding the targeting adaptor;
(c) administering to a subject a gating adaptor; wherein the targeting adaptor comprises a first ligand-binding domain (tLBD-1) specific for a cell-surface antigen and a second ligand-binding domain (tLBD-2) specific for the gating adaptor; and wherein the chimeric receptor comprises an extracellular ligand-binding domain (rLBD) specific for the gating adaptor, a transmembrane domain, and an intracellular actuator domain; and wherein the method generates an effective amount of the GATR system in the subject.

### Vectors and Pharmaceutical Compositions Thereof

The present disclosure provides an isolated cell, comprising:
(a) a polynucleotide encoding a targeting adaptor, and/or a targeting adaptor comprising a transmembrane domain and expressed on the surface of the isolated cell; and
(b) a polynucleotide encoding a chimeric receptor, and/or a chimeric receptor expressed on the surface of the isolated cell, wherein the targeting adaptor comprises a first ligand-binding domain (tLBD-1) specific for a cell-surface antigen and a second ligand-binding domain (tLBD-2) specific for the gating adaptor; and wherein the chimeric receptor comprises an extracellular ligand-binding domain (rLBD) specific for the gating adaptor, a transmembrane domain, and an intracellular actuator domain.

### Methods of Using Vectors for In Vivo Engineering of Immune Cells

The present disclosure provides a targeting adaptor, comprising a first ligand-binding domain (tLBD-1) specific for a cell-surface antigen and a second ligand-binding domain (tLBD-2) specific for a moiety selected from folate, a CA9 ligand, acetazolamide, fluorescein, rapamycin, a rapalog or a derivative thereof.

In some embodiments, the tLBD-1 of the targeting adaptor comprises an antibody, antigen-binding fragment thereof or a single-chain variable fragment (scFv).

In some embodiments, the tLBD-2 of the targeting adaptor comprises an antibody, antigen-binding fragment thereof or a single-chain variable fragment (scFv).

In some embodiments, the tLBD-2 comprises a folate receptor domain.

In some embodiments, the folate receptor domain is a folate receptor alpha (FRα) domain.

In some embodiments, the FRα domain shares at least 80%, at least 85%, at least 90% or at least 95% identity to:

(SEQ ID NO: 18)
GSSRTELLNVCMNAKHHKEKPGPEDKLHEQCRPWRKNACCSTNTSQEAHK
DVSYLYRFNWNHCGEMAPACKRHFIQDTCLYECSPNLGPWIQQVDQSWRK
ERVLNVPLCKEDCEQWWEDCRTSYTCKSNWHKGWNWTSGFNKCAVGAACQ
PFHFYFPTPTVLCNEIWTHSYKVSNYSRGSGRCIQMWFDPAQGNPNEEVA
RFYAAAMS

In some embodiments, the tLBD-2 comprises a carbonic anhydrase IX (CA9) domain.

In some embodiments, the CA9 domain shares at least 80%, at least 85%, at least 90% or at least 95% identity to:

(SEQ ID NO: 19)
HHWRYGGDPPWPRVSPACAGRFQSPVDIRPQLAAFSPALRPLELLGFQLP
PLPELRLRNNGHSVQLTLPPGLEMALGPGREYRALQLHLHWGAAGRPGSE
HTVEGHRFPAEIHVVHLSTAFARVDEALGRPGGLAVLAAFLEEGPEENSA
YEQLLSRLEEIAEEGSETQVPGLDISALLPSDFSRYFQYEGSLTTPPCAQ
GVIWTVFNQTVMLSAKQLHTLSDTLWGPGDSRLQLNFRATQPLNGRVIEA
SFPAGVD

In some embodiments, the CA9 domain shares at least 80%, at least 85%, at least 90% or at least 95% identity to:

(SEQ ID NO: 20)
HWRYGGDPPWPRVSPACAGRFQSPVDIRPQLAAFSPALRPLELSGFQLPP
LPELRLRNNGHSVQLTLPPGLEMKLGPGREYRALQLHLHWGAAGRPGSEH
TVEGHRFPAEIHVVHLSTKYARVDEALGRPGGLAVLAAFLEEGPEENSAY
EQLLSRLEEIAEEGSETQVPGLDISALLPSDFSRYFQYEGSLTTPPCAQG
VIWTVFNQTVSLSAKQLHTLSDTLWGPGDSRLQLNFRATQPLNGRVIEAS
FPAGVD

Some embodiments of the present disclosure describe a vector comprising a polynucleotide encoding a targeting adaptor.

In some embodiments, the targeting adaptor comprises a first ligand-binding domain (tLBD-1) specific for a cell-surface antigen, a second ligand-binding domain (tLBD-2) specific for the gating adaptor.

In some embodiments, the targeting adaptor comprises a first ligand-binding domain (tLBD-1) specific for a cell-surface antigen, a second ligand-binding domain (tLBD-2) specific for the gating adaptor and a transmembrane domain.

### Kits

The present disclosure provides a kit comprising the GATR system comprising any of the compositions of the present disclosure and a package insert comprising instructions for using the GATR system.

The present disclosure provides a kit comprising the GATR system comprising a vector for use with a gating adaptor which comprises:
(a) a polynucleotide encoding a targeting adaptor; and
(b) a polynucleotide encoding a chimeric receptor,
wherein the targeting adaptor comprises a first ligand-binding domain (tLBD-1) specific for a cell-surface antigen and a second ligand-binding domain (tLBD-2) specific for the gating adaptor; and wherein the chimeric receptor comprises an extracellular ligand-binding domain (rLBD) specific for the gating adaptor, a transmembrane domain, and an intracellular actuator domain; and a package insert comprising instructions for using the GATR system.

The present disclosure provides a kit comprising engineered immune cell further comprising a chimeric receptor.

The present disclosure provides a kit comprising engineered immune cell further comprising a vector which encodes a chimeric receptor.

The present disclosure provides a kit comprising the GATR system comprising a vector for use with a gating adaptor comprises:
(a) a polynucleotide encoding a targeting adaptor; and
(b) a polynucleotide encoding a chimeric receptor,
wherein the targeting adaptor comprises a first ligand-binding domain (tLBD-1) specific for a cell-surface antigen, a second ligand-binding domain (tLBD-2) specific for the gating adaptor and a transmembrane domain; and wherein the chimeric receptor comprises an extracellular ligand-binding domain (rLBD) specific for the gating adaptor, a transmembrane domain, and an intracellular actuator domain; and a package insert comprising instructions for using the GATR system.

The present disclosure provides a kit comprising an engineered immune cell comprising a chimeric receptor, wherein the chimeric receptor comprises an extracellular ligand-binding domain (rLBD) specific for the gating adaptor, a transmembrane domain, and an intracellular actuator domain.

The present disclosure provides a kit comprising an engineered immune cell comprising a chimeric receptor, wherein the chimeric receptor is encoded by a vector.

### Retroviral Particles

Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, e.g. p7, p24, and p17, containing a retroviral or lentiviral envelope including one or more of the env encoded glycoproteins, e.g. p41, p120, and p160, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to the gating adaptor. In some embodiments, a sequence encoding a receptor that specifically binds to the gating adaptor is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise a polynucleotide comprising a sequence encoding a T cell activator protein. In some embodiments, the retroviral particles comprise a polynucleotide comprising a sequence encoding a targeting adaptor-binding receptor. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR.

In some embodiments, the retroviral particles comprise a cell surface receptor that binds to a ligand on a target host cell, allowing host cell transduction. The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof. In some embodiments, the viral fusion glycoprotein comprises the amino acid sequence of SEQ ID NO: 65 (Cocal G protein). In some embodiments, the viral fusion glycoprotein comprises an amino acid sequence at least 95% identical to SEQ ID NO: 65 (Cocal G protein). In some embodiments, the viral fusion glycoprotein comprises an amino acid sequence at least 50%, at least 55%, at least 60%, at least 65%, at least 70%, at least 75%, at least 80%, at least 85%, at least 90%, at least 95%, at least 96%, at least 97%, at least 98%, or at least 99% identical to SEQ ID NO: 65 (Cocal G protein).

(SEQ ID NO: 65)
NFLLLTFIVLPLCSHAKFSIVFPQSQKGNWKNVPSSYHYCPSSSDQNWHN
DLLGITMKVKMPKTHKAIQADGWMCHAAKWITTCDFRWYGPKYITHSIHS
IQPTSEQCKESIKQTKQGTWMSPGFPPQNCGYATVTDSVAVVVQATPHHV
LVDEYTGEWIDSQFPNGKCETEECETVHNSTVWYSDYKVTGLCDATLVDT
EITFFSEDGKKESIGKPNTGYRSNYFAYEKGDKVCKMNYCKHAGVRLPSG
VWFEFVDQDVYAAAKLPECPVGATISAPTQTSVDVSLILDVERILDYSLC
QETWSKIRSKQPVSPVDLSYLAPKNPGTGPAFTIINGTLKYFETRYIRID
IDNPIISKMVGKISGSQTERELWTEWFPYEGVEIGPNGILKTPTGYKFPL
FMIGHGMLDSDLHKTSQAEVFEHPHLAEAPKQLPEEETLFFGDTGISKNP
VELIEGWFSSWKSTVVTFFFAIGVFILLYVVARIVIAVRYRYQGSNNKRI
YNDIEMSRFRK

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD 114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezuelan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors.

In some embodiments, a polynucleotide of the present disclosure may encode a targeting adaptor comprising any of the targeting adaptors of the present disclosure.

In some embodiments, a vector of the present disclosure comprises a polynucleotide which encodes a targeting adaptor comprising any of the targeting adaptors of the present disclosure.

In some embodiments of the present disclosure, a vector for use with a gating adaptor comprises:
(a) a polynucleotide encoding a targeting adaptor; and
(b) a polynucleotide encoding a chimeric receptor,
wherein the targeting adaptor comprises a first ligand-binding domain (tLBD-1) specific for a cell-surface antigen and a second ligand-binding domain (tLBD-2) specific for the gating adaptor; and wherein the chimeric receptor comprises an extracellular ligand-binding domain (rLBD) specific for the gating adaptor, a transmembrane domain, and an intracellular actuator domain.

In some embodiments of the present disclosure, a vector for use with a gating adaptor comprises:
(a) a polynucleotide encoding a targeting adaptor; and
(b) a polynucleotide encoding a chimeric receptor, wherein the targeting adaptor comprises a first ligand-binding domain (tLBD-1) specific for a cell-surface antigen, a second ligand-binding domain (tLBD-2) specific for the gating adaptor and a transmembrane domain; and wherein the chimeric receptor comprises an extracellular ligand-binding domain (rLBD) specific for the gating adaptor, a transmembrane domain, and an intracellular actuator domain.

In some embodiments, the vector may be a viral vector, a retroviral vector, a lentiviral vector, a gamma-retroviral vector. In some embodiments, the viral vector comprises a VSV G-protein or functional variant thereof. In some embodiments, the viral vector comprises a Cocal G-protein or functional variant thereof.

### Nucleic Acid Vectors

As used herein, the term “nucleic acid vector” is intended to mean any nucleic acid that functions to carry, harbor or express a nucleic acid of interest. Nucleic acid vectors can have specialized functions such as expression, packaging, pseudotyping, transduction or sequencing, for example. Nucleic acid vectors also can have, for example, manipulatory functions such as a cloning or shuttle vector. The structure of the vector can include any desired form that is feasible to make and desirable for a particular use. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

Non-limiting examples of vector systems of the present disclosure include a retrovirus, a lentivirus, a foamy virus, and a Sleeping Beauty transposon.

### Transduction Enhancers

In some embodiments, viral particles according to the present disclosure comprise transduction enhancers.

A “transduction enhancer” as used herein refers to a transmembrane protein that activates T cells. Transduction enhancers may be incorporated into the viral envelopes of viral particles according to the present disclosure. The transduction enhancer may comprise a mitogenic and/or cytokine-based domain. The transduction enhancer may comprise T cell activation receptors, NK cell activation receptors, co-stimulatory molecules, or portions thereof.

### Mitogenic Transduction Enhancers

The viral vector of the present invention may comprise a mitogenic transduction enhancer in the viral envelope. In some embodiments, the mitogenic transduction enhancer is derived from the host cell during retroviral vector production. In some embodiments, the mitogenic transduction enhancer is made by the packaging cell and expressed at the cell surface. When the nascent retroviral vector buds from the host cell membrane, the mitogenic transduction enhancer may be incorporated in the viral envelope as part of the packaging cell-derived lipid bilayer.

In some embodiments, the transduction enhancer is host-cell derived. The term “host-cell derived” indicates that the mitogenic transduction enhancer is derived from the host cell as described above and is not produced as a fusion or chimera from one of the viral genes, such as gag, which encodes the main structural proteins; or env, which encodes the envelope protein.

Envelope proteins are formed by two subunits, the transmembrane (TM) that anchors the protein into the lipid membrane and the surface (SU) which binds to the cellular receptors. In some embodiments, the packaging-cell derived mitogenic transduction enhancer of the present invention does not comprise the surface envelope subunit (SU).

The mitogenic transduction enhancer may have the structure: M-S-TM, in which M is a mitogenic domain; S is an optional spacer domain and TM is a transmembrane domain.

### Transduction Enhancer Mitogenic Domains

The mitogenic domain is the part of the mitogenic transduction enhancer which causes T-cell activation. It may bind or otherwise interact, directly or indirectly, with a T cell, leading to T cell activation. In particular, the mitogenic domain may bind a T cell surface antigen, such as CD3, CD28, CD134 and CD137.

CD3 is a T-cell co-receptor. It is a protein complex composed of four distinct chains. In mammals, the complex contains a CD3y chain, a CD35 chain, and two CD3e chains. These chains associate with the T-cell receptor (TCR) and the ζ-chain to generate an activation signal in T lymphocytes. The TCR, ζ-chain, and CD3 molecules together comprise the TCR complex.

In some embodiments, the mitogenic domain may bind to a CD3 ε chain.

CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD134, also known as OX40, is a member of the TNFR-superfamily of receptors which is not constitutively expressed on resting naive T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels.

CD137, also known as 4-1BB, is a member of the tumor necrosis factor (TNF) receptor family. CD137 can be expressed by activated T cells, but to a larger extent on CD8 than on CD4 T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity.

The mitogenic domain may comprise all or part of an antibody or other molecule which specifically binds a T-cell surface antigen. The antibody may activate the TCR or CD28. The antibody may bind the TCR, CD3 or CD28. Examples of such antibodies include: OKT3, 15E8 and TGN1412. Other suitable antibodies include:
Anti-CD28: CD28.2, 10F3
Anti-CD3/TCR: UCHT1, YTH12.5, TR66

The mitogenic domain may comprise the binding domain from OKT3, 15E8, TGN1412, CD28.2, 10F3, UCHT1, YTH12.5 or TR66.

The mitogenic domain may comprise all or part of a co-stimulatory molecule such as OX40L and 41 BBL. For example, the mitogenic domain may comprise the binding domain from OX40L or 41 BBL.

OKT3, also known as Muromonab-CD3 is a monoclonal antibody targeted at the CD3e chain. It is clinically used to reduce acute rejection in patients with organ transplants. It was the first monoclonal antibody to be approved for clinical use in humans. The CDRs of OKT3 are as follows:

CDRH1:
(SEQ ID NO: 66)
GYTFTRY
CDRH2:
(SEQ ID NO: 67)
NPSRGY
CDRH3:
(SEQ ID NO: 68)
YYDDHYCLDY
CDRL1:
(SEQ ID NO: 69)
SASSSVSYMN
CDRL2:
(SEQ ID NO: 70)
DTSKLAS
CDRL3:
(SEQ ID NO: 71)
QQWSSNPFT

15E8 is a mouse monoclonal antibody to human CD28. Its CDRs are as follows:

CDRH1:
(SEQ ID NO: 72)
GFSLTSY
CDRH2:
(SEQ ID NO: 73)
WAGGS
CDRH3:
(SEQ ID NO: 74)
DKRAPGKLYYGYPDY
CDRL1:
(SEQ ID NO: 75)
RASESVEYYVTSLMQ
CDRL2:
(SEQ ID NO: 76)
AASNVES
CDRL3:
(SEQ ID NO: 77)
QQTRKVPST

TGN1412 (also known as CD28-SuperMAB) is a humanized monoclonal antibody that not only binds to, but is a strong agonist for, the CD28 receptor. Its CDRs are as follows.

CDRH1:
(SEQ ID NO: 78)
GYTFSY
CDRH2:
(SEQ ID NO: 79)
YPGNVN
CDRH3:
(SEQ ID NO: 80)
SHYGLDWNFDV
CDRL1:
(SEQ ID NO: 81)
HASQNIYVLN
CDRL2:
(SEQ ID NO: 82)
KASNLHT
CDRL3:
(SEQ ID NO: 83)
QQGQTYPYT

OX40L is the ligand for CD134 and is expressed on such cells as DC2s (a subtype of dendritic cells) enabling amplification of Th2 cell differentiation. OX40L has also been designated CD252 (cluster of differentiation 252).

OX40L sequence
(SEQ ID NO: 84)
MERVQPLEENVGNAARPRFERNKLLLVASVIQGLGLLLCFTYICLHFSAL
QVSHRYPRIQSIKVQFTEYKKEKGFILTSQKEDEIMKVQNYLISLKGYFS
QEVNISLHYQKDEEPLFQLKKVRSVNSLMVASLTYKDKVYLNVTTDNTSL
DDFHVNGGELILIHQNPGEFCVL

4-1BBL is a cytokine that belongs to the tumor necrosis factor (TNF) ligand family. This transmembrane cytokine is a bidirectional signal transducer that acts as a ligand for 4-1BB, which is a costimulatory receptor molecule in T lymphocytes. 4-1BBL has been shown to reactivate anergic T lymphocytes in addition to promoting T lymphocyte proliferation.

4-1BBL sequence
(SEQ ID NO: 85)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

### Transduction Enhancer Spacer Domains

The mitogenic transduction enhancer and/or cytokine-based transduction enhancer may comprise a spacer sequence to connect the antigen-binding domain with the transmembrane domain. A flexible spacer allows the antigen-binding domain to orient in different directions to facilitate binding.

The spacer sequence may, for example, comprise an lgG1 Fc region, an lgG1 hinge or a human CD8 stalk or the mouse CD8 stalk. The spacer may alternatively comprise an alternative linker sequence which has similar length and/or domain spacing properties as an lgG1 Fc region, an lgG1 hinge or a CD8 stalk. A human lgG1 spacer may be altered to remove Fc binding motifs.

Examples of amino acid sequences for these spacers are given below:

SEQ ID NO: 86 (hinge-CH2CH3 of human IgG1)
AEPKSPDKTHTCPPCPAPPVAGPSVFLFPPKPKDTL
MIARTPEVTCWVDVSHEDPEVKFNWYVDGVEVHNAK
TKPREEQYNSTYRVVSVLTVLHQDWLNGKEYKCKVS
NKALPAPIEKTISKAKGQPREPQVYTLPPSRDELTK
NQVSLTCLVKGFYPSDIAVEWESNGQPENNYKTTPP
VLDSDGSFFLYSKLTVDKSRWQQGNVFSCSVMHEAL
HNHYTQK SLSLSPGKKD
SEQ ID NO: 87 (human CD8 stalk):
TTTPAPRPPTPAPTIASQPLSLRPEACRPAAGGAVH
TRGLDFACDI
SEQ ID NO: 88 (human lgG1 hinge):
AEPKSPDKTHTCPPCPKDPK
SEQ ID NO: 89 (CD2 ectodomain):
KEITNALETWGALGQDINLDIPSFQMSDDIDDIKWE
KTSDKKKIAQFRKEKETFKEKDTYKLFKNGTLKIKH
LKTDDQDIYKVSIYDTKGKNVLEKIFDLKIQERVSK
PKISWTCINTTLTCEVMNGTDPELNLYQDGKHLKLS
QRVITHKWTTSLSAKFKCTAGNKVSKESSV EPVSC
PEKGLD
SEQ ID NO: 90 (CD34 ectodomain):
SLDNNGTATPELPTQGTFSNVSTNVSYQETTTPSTL
GSTSLHPVSQHGNEATTNITETTVKFTSTSVITSVY
GNTNSSVQSQTSVISTVFTTPANVSTPETTLKPSLS
PGNVSDLSTTSTSLATSPTKPYTSSSPILSDIKAEI
KCSGIREVKLTQGICLEQNKTSSCAEFKKDRGEGLA
RVLCGEEQADADAGAQVCSLLLAQSEVRPQCLLLVL
ANRTEISSKLQLMK KHQSDLKKLGI LDFTEQDVA
SHQSYSQKT

In some embodiments, the spacer sequence may be derived from a human protein.

### Transduction Enhancer Transmembrane Domains

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

An alternative option to a transmembrane domain is a membrane-targeting domain such as a GPI anchor. GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane. In some embodiments, the present tagging protein comprises a GPI signal sequence.

### Cytokine-Based Transduction Enhancers

The viral vector of the present invention may comprise a cytokine-based transduction enhancer in the viral envelope. In some embodiments, the cytokine-based transduction enhancer is derived from the host cell during viral vector production. In some embodiments, the cytokine-based transduction enhancer is made by the host cell and expressed at the cell surface. When the nascent viral vector buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain and TM is the transmembrane domain. The spacer domain and transmembrane domains are as defined above.

### Transduction Enhancer Cytokine Domains

The cytokine domain may comprise part or all of a T-cell activating cytokine, such as from IL2, IL7 and IL15. The cytokine domain may comprise part of the cytokine, as long as it retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding. The sequence of IL2 is shown as

SEQ ID NO: 91:
MYRMQLLSCIALSLALVTNSAPTSSSTKKTQLQLEH
LLLDLQMILNGINNYKNPKLTRMLTFKFYMPKKATE
LKHLQCLEEELKPLEEVLNLAQSKNFHLRPRDLISN
INVIVLELK GSETTFMCEYADETATIVEFLNRWIT
FCQSIISTLT

IL7 is a cytokine that serves as a growth factor for early lymphoid cells of both B- and T-cell lineages. The sequence of IL7 is shown as SEQ ID NO: 92:

SEQ ID NO: 92:
MFHVSFRYIFGLPPLILVLLPVASSDCDIEGKDGKQ
YESVLMVSIDQLLDSMKEIGSNCLNNEFNFFKRHIC
DANKEGMFLFRAARKLRQFLKMNSTGDFDLHLLKVS
EGTTILLNCTGQVKGRKPAALGEAQPTKSLEENKSL
KEQKKLNDLCFLKRLLQEIKTCWNKILM GTKEH

IL15 is a cytokine with structural similarity to IL-2. Like IL-2, IL-15 binds to and signals through a complex composed of IL-2/IL-15 receptor beta chain and the common gamma chain. IL-15 is secreted by mononuclear phagocytes, and some other cells, following infection by virus(es). This cytokine induces cell proliferation of natural killer cells; cells of the innate immune system whose principal role is to kill virally infected cells. The sequence of IL-15 is shown as SEQ ID NO: 93:

SEQ ID NO: 93:
MRISKPHLRSISIQCYLCLLLNSHFLTEAGIHVFIL
GCFSAGLPKTEANWVNVISDLKKIEDLIQSMHIDAT
LYTESDVHPSCKVTAMKCFLLELQVISLESGDASIH
DTVENLIILANN SLSSNGNVTESGCKECEELEEKN
IKEFLQSFVHIVQMFINTS

The cytokine-based transduction enhancer may comprise one of the following sequences, or a variant thereof:

(membrane-IL7)
SEQ ID NO: 94
MAHVSFRYIFGLPPLILVLLPVASSDCDIEGKDGKQ
YESVLMVSIDQLLDSMKEIGSNCLNNEFNFFKRHIC
DANKEGMFLFRAARKLRQFLKMNSTGDFDLHLLKVS
EGTTILLNCTGQVKGRKPAALGEAQPTKSLEENKSL
KEQKKLNDLCFLKRLLQEIKTCWNKILMGTKEHSGG
GSPAKPTTTPAPRPPTPAPTIASQPLSLRPEACRPA
AGGAVHTRGLDF ACDIYIWAPLAGTCGVLLLSLVI
TLYCNHRNRRRVCKCPRPVV;
(membrane-IL15)
SEQ ID NO: 95
MGLVRRGARAGPRMPRGWTALCLLSLLPSGFMAGIH
VFILGCFSAGLPKTEANWVNVISDLKKIEDLIQSMH
IDATLYTESDVHPSCKVTAMKCFLLELQVISLESGD
ASIHDTVENLIILANNSLSSNGNVTESGCKECEELE
EKNIKEFLQSFVHIVQMFINTSSPAKPTTTPAPRPP
TPAPTIASQPLSLRPEACRPAAGGAVHTRGLDFACD
IYIWAPLAGTCGVLLLS LVITLYCNHRNRRRVCKC
PRPVV.

The cytokine-based transduction enhancer may comprise a variant of the sequence shown as SEQ ID NOs 94 or 95 having at least 80, 85, 90, 95, 98 or 99% sequence identity, provided that the variant sequence is a cytokine-based transduction enhancer having the required properties i.e. the capacity to activate a T cell when present in the envelope protein of a retroviral or lentiviral vector.

### Illustrative Advantages of Transduction Enhancers

In some embodiments, the present disclosure provides a viral vector with a built-in transduction enhancer. The vector may have the capability to both stimulate the T-cell and to also effect gene insertion. This may produce one or more advantages, including: (1) simplifying the process of T-cell engineering, as only one component needs to be added; (2) avoiding removal of beads and the associated reduction in yield as the virus is labile and does not have to be removed; (3) reducing the cost of T-cell engineering as only one component needs to be manufactured; (4) allowing greater design flexibility, as each T-cell engineering process will involve making a gene-transfer vector, the same product can also be made with a transduction enhancer to “fit” the product; (5) shortening the production process: in soluble antigen/bead-based approaches the mitogen and the vector are typically given sequentially separated by one, two or sometimes three days, this can be avoided with the retroviral vector of the present invention since transduction enhancement and viral entry are synchronized and simultaneous; (6) simplifying engineering as there is no need to test a lot of different fusion proteins for expression and functionality; (7) allowing for the possibility to add more than one signal at the same time; and (8) allowing for the regulation of the expression and/or expression levels of each signal/protein separately.

### Illustrative Embodiments of Viral Vectors Comprising Transduction Enhancers

In some embodiments, the viral envelope comprises one or more transduction enhancers. In some embodiments, the transduction enhancers include T cell activation receptors, NK cell activation receptors, and/or co-stimulatory molecules. In some embodiments, one or more transduction enhancers comprise one or more of anti-CD3scFv, CD86, and CD137L. In some embodiments, the transduction enhancers comprise every one of anti-CD3 scFv, CD86, and CD137L.

In some embodiments, the transduction enhancer comprises a mitogenic stimulus, and/or a cytokine stimulus, which is incorporated into a retroviral or lentiviral capsid, such that the virus both activates and transduces T cells. This removes the need to add vector, mitogen and cytokines separately. In some embodiments, the transduction enhancer comprises a mitogenic transmembrane protein and/or a cytokine-based transmembrane protein that is included in the producer or packaging cell, which get(s) incorporated into the retrovirus when it buds from the producer/packaging cell membrane. In some embodiments, the transduction enhancers are expressed as separate cell surface molecules on the producer cell rather than being part of the viral envelope glycoprotein.

In some embodiments, the present disclosure provides a retroviral or lentiviral vector having a viral envelope which comprises:
(i) a mitogenic transduction enhancer which comprises a mitogenic domain and a transmembrane domain; and/or
(ii) a cytokine-based transduction enhancer which comprises a cytokine domain and a transmembrane domain.

In some embodiments, the transduction enhancers are not part of a viral envelope glycoprotein. In some embodiments, the retroviral or lentiviral vector comprises a separate viral envelope glycoprotein, encoded by an env gene. Since the mitogenic stimulus and/or cytokine stimulus are provided on a molecule which is separate from the viral envelope glycoprotein, integrity of the viral envelope glycoprotein is maintained and there is no negative impact on viral titer.

In some embodiments, there is provided a retroviral or lentiviral vector having a viral envelope which comprises:
(i) a viral envelope glycoprotein: and
(ii) a mitogenic transduction enhancer having the structure: M-S-TM
in which M is a mitogenic domain; S is an optional spacer and TM is a transmembrane domain; and/or
(iii) a cytokine-based transduction enhancer which comprises a cytokine domain and a transmembrane domain.

In some embodiments, the mitogenic transduction enhancer and/or cytokine-based transduction enhancer are not part of the viral envelope glycoprotein. In some embodiments, they exist as separate proteins in the viral envelope and are encoded by separate genes. In some embodiments, the mitogenic transduction enhancer has the structure:

M-S-TM
in which M is a mitogenic domain; S is an optional spacer and TM is a transmembrane domain.

In some embodiments, the mitogenic transduction enhancer binds an activating T-cell surface antigen. In some embodiments, the antigen is CD3, CD28, CD134 or CD137. The mitogenic transduction enhancer may comprise an agonist for such an activating T-cell surface antigen.

The mitogenic transduction enhancer may comprise the binding domain from an antibody such as OKT3, 15E8, TGN1412; or a costimulatory molecule such as OX40L or 41 BBL. The viral vector may comprise two or more mitogenic transduction enhancers in the viral envelope. For example, the viral vector may comprise a first mitogenic transduction enhancer which binds CD3 and a second mitogenic transduction enhancer which binds CD28. The cytokine-based transduction enhancer may, for example, comprise a cytokine selected from IL2, IL7 and IL15.

In some embodiments, there is provided a retroviral or lentiviral vector having a viral envelope which comprises:
(a) a first mitogenic transduction enhancer which binds CD3; and
(b) a second mitogenic transduction enhancer which binds CD28.

In some embodiments, there is provided a retroviral or lentiviral vector having a viral envelope which comprises:
(a) a first mitogenic transduction enhancer which binds CD3;
(b) a second mitogenic transduction enhancer which binds CD28; and
(c) a cytokine-based transduction enhancer which comprises IL2.

In some embodiments, there is provided a retroviral or lentiviral vector having a viral envelope which comprises:
(a) a first mitogenic transduction enhancer which binds CD3;
(b) a second mitogenic transduction enhancer which binds CD28;
(c) a cytokine-based transduction enhancer which comprises IL7; and (d) a cytokine-based transduction enhancer which comprises IL15.

### Further Numbered Embodiments

Further numbered embodiments of the present disclosure are provided as follows:
Embodiment 1. A Gated Adaptor Targeting Receptor (GATR) system, comprising:
(a) a gating adaptor;
(b) a targeting adaptor, or a vector encoding the targeting adaptor; and
(c) an engineered immune cell comprising a chimeric receptor, optionally a chimeric antigen receptor, or a vector encoding the chimeric receptor,
wherein the targeting adaptor comprises a first ligand-binding domain (tLBD-1) specific for a cell-surface antigen and a second ligand-binding domain (tLBD-2) specific for the gating adaptor; and
wherein the chimeric receptor comprises an extracellular ligand-binding domain (rLBD) specific for the gating adaptor, a transmembrane domain, and an intracellular actuator domain.
Embodiment 2. The system of Embodiment 1, wherein the rLBD comprises an antibody or antigen-binding fragment thereof.
Embodiment 3. The system of Embodiment 2, wherein the rLBD comprises a single-chain variable fragment (scFv).
Embodiment 4. The system of Embodiment 1, wherein the rLBD comprises a T-cell receptor (TCR) or antigen-binding fragment thereof.
Embodiment 5. The system of any one of Embodiments 1 to 4, wherein the tLBD-1 comprises an antibody or antigen-binding fragment thereof.
Embodiment 6. The system of Embodiment 5, wherein the tLBD-1 comprises a single-chain variable fragment (scFv).
Embodiment 7. The system of any one of Embodiments 1 to 4, wherein the tLBD-1 comprises a T-cell receptor (TCR) or antigen-binding fragment thereof.
Embodiment 8. The system of any one of Embodiments 1 to 7, wherein the tLBD-2 comprises an antibody or antigen-binding fragment thereof.
Embodiment 9. The system of any one of Embodiments 1 to 7, wherein the tLBD-2 comprises a folate receptor domain.
Embodiment 10. The system of Embodiment 9, wherein the folate receptor domain is a folate receptor alpha (FRα) domain.
Embodiment 11. The system of Embodiment 10, wherein the FRα domain shares at least 95% identity to:

(SEQ ID NO: 18)
GSSRTELLNVCMNAKHHKEKPGPEDKLHEQCRPWRK
NACCSTNTSQEAHKDVSYLYRFNWNHCGEMAPACKR
HFIQDTCLYECSPNLGPWIQQVDQSWRKERVLNVPL
CKEDCEQWWEDCRTSYTCKSNWHKGWNWTSGFNKCA
VGAACQPFHFYFPTPTVLCNEIWTHSYKVSNYSRGS
GRCIQMWFDPAQGNPNEEVARFYAAAMS.

Embodiment 12. The system of any one of Embodiments 1 to 7, wherein the tLBD-2 comprises a carbonic anhydrase IX (CA9) domain.
Embodiment 13. The system of Embodiment 12, wherein the CA9 domain shares at least 95% identity to:

(SEQ ID NO: 19)
HHWRYGGDPPWPRVSPACAGRFQSPVDIRPQLAAFS
PALRPLELLGFQLPPLPELRLRNNGHSVQLTLPPGL
EMALGPGREYRALQLHLHWGAAGRPGSEHTVEGHRF
PAEIHVVHLSTAFARVDEALGRPGGLAVLAAFLEEG
PEENSAYEQLLSRLEEIAEEGSETQVPGLDISALLP
SDFSRYFQYEGSLTTPPCAQGVIWTVFNQTVMLSAK
QLHTLSDTLWGPGDSRLQLNFRATQPLNGRVIEASF
PAGVD.

Embodiment 14. The system of Embodiment 12, wherein the CA9 domain shares at least 95% identity to:

(SEQ ID NO: 20)
HWRYGGDPPWPRVSPACAGRFQSPVDIRPQLAAFSP
ALRPLELSGFQLPPLPELRLRNNGHSVQLTLPPGLE
MKLGPGREYRALQLHLHWGAAGRPGSEHTVEGHRFP
AEIHVVHLSTKYARVDEALGRPGGLAVLAAFLEEGP
EENSAYEQLLSRLEEIAEEGSETQVPGLDISALLPS
DFSRYFQYEGSLTTPPCAQGVIWTVFNQTVSLSAKQ
LHTLSDTLWGPGDSRLQLNFRATQPLNGRVIEASFP
AGVD.

Embodiment 15. The system of any one of Embodiments 1 to 14, wherein the gating adaptor is a small molecule.
Embodiment 16. The system of Embodiment 15, wherein the gating adaptor comprises a first moiety recognized by rLBD and a second moiety recognized by tLBD-2.
Embodiment 17. The system of Embodiment 16, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 18. The system of Embodiment 17, wherein the first moiety is fluorescein or a derivative thereof.
Embodiment 19. The system of any one of Embodiments 16 to 18, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 20. The system of Embodiment 19, wherein the second moiety is folate or a derivative thereof.
Embodiment 21. The system of Embodiment 19, wherein the second moiety is a CA9 ligand or a derivative thereof.
Embodiment 22. The system of Embodiment 20, wherein the gating adaptor is a folate-fluorescein conjugate.
Embodiment 23. The system of Embodiment 19, wherein the gating adaptor is a CA9 ligand-fluorescein conjugate.
Embodiment 24. The system of any one of Embodiments 1 to 23, wherein the chimeric receptor comprises one polypeptide chain.
Embodiment 25. The system of any one of Embodiments 1 to 23, wherein the chimeric receptor comprises at least two polypeptide chains.
Embodiment 26. The system of any one of Embodiments 1 to 25, wherein the chimeric receptor specifically binds fluorescein.
Embodiment 27. The system of Embodiment 26, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.
Embodiment 28. The system of Embodiment 26, wherein the rLBD comprises a

(SEQ ID NO: 31)
SVLTQPSSVSAAPGQKVTISCSGSTSNIGNNYVSWY
QQHPGKAPKLMIYDVSKRPSGVPDRFSGSKSGNSAS
LDISGLQSEDEADYYCAAWDDSLSEFLFGTGTKLTV
LG;

polypeptide sequence sharing at least 95% identity to:

(SEQ ID NO: 32)
QVQLVESGGNLVQPGGSLRLSCAASGFTFGSFSMSW
VRQAPGGGLEWVAGLSARSSLTHYADSVKGRFTISR
DNAKNSVYLQMNSLRVEDTAVYYCARRSYDSSGYWG
HFYSYMDVWGQGTLVTVSS.

Embodiment 29. The system of Embodiment 28, wherein the rLBD shares at least 95% identity to:

(SEQ ID NO: 30)
SVLTQPSSVSAAPGQKVTISCSGSTSNIGNNYVSWY
QQHPGKAPKLMIYDVSKRPSGVPDRFSGSKSGNSAS
LDISGLQSEDEADYYCAAWDDSLSEFLFGTGTKLTV
LGSTSGSGKPGSGEGSTKGQVQLVESGGNLVQPGGS
LRLSCAASGFTFGSFSMSWVRQAPGGGLEWVAGLSA
RSSLTHYADSVKGRFTISRDNAKNSVYLQMNSLRVE
DTAVYYCARRSYDSSGYWGHFYSYMDVWGQGTLVTV
SS.

Embodiment 30. The system of Embodiment 29, wherein the chimeric receptor shares at least 95% identity to:

(SEQ ID NO: 58)
SVLTQPSSVSAAPGQKVTISCSGSTSNIGNNYVSWY
QQHPGKAPKLMIYDVSKRPSGVPDRFSGSKSGNSAS
LDISGLQSEDEADYYCAAWDDSLSEFLFGTGTKLTV
LGSTSGSGKPGSGEGSTKGQVQLVESGGNLVQPGGS
LRLSCAASGFTFGSFSMSWVRQAPGGGLEWVAGLSA
RSSLTHYADSVKGRFTISRDNAKNSVYLQMNSLRVE
DTAVYYCARRSYDSSGYWGHFYSYMDVWGQGTLVTV
SSESKYGPPCPPCPMFWVLVVVGGVLACYSLLVTVA
FIIFWVKRGRKKLLYIFKQPFMRPVQTTQEEDGCSC
RFPEEEEGGCELRVKFSRSADAPAYQQGQNQLYNEL
NLGRREEYDVLDKRRGRDPEMGGKPRRKNPQEGLYN
ELQKDKMAEAYSEIGMKGERRRGKGHDGLYQGLSTA
TKDTYDALHMQALPPR.

Embodiment 31. The system of any one of Embodiments 1 to 30, wherein the targeting adaptor shares at least 95% identity to:

(SEQ ID NO: 59)
GSSRTELLNVCMNAKHHKEKPGPEDKLHEQCRPWRK
NACCSTNTSQEAHKDVSYLYRFNWNHCGEMAPACKR
HFIQDTCLYECSPNLGPWIQQVDQSWRKERVLNVPL
CKEDCEQWWEDCRTSYTCKSNWHKGWNWTSGFNKCA
VGAACQPFHFYFPTPTVLCNEIWTHSYKVSNYSRGS
GRCIQMWFDPAQGNPNEEVARFYAAAMSESKYGPPC
PPCPGGRMALIVLGGVAGLLLFIGLGIFFCVRCRHR
RRQ.

Embodiment 32. The system of any one of Embodiments 1 to 30, wherein the targeting adaptor shares at least 95% identity to:

(SEQ ID NO: 18)
GSSRTELLNVCMNAKHHKEKPGPEDKLHEQCRPWRK
NACCSTNTSQEAHKDVSYLYRFNWNHCGEMAPACKR
HFIQDTCLYECSPNLGPWIQQVDQSWRKERVLNVPL
CKEDCEQWWEDCRTSYTCKSNWHKGWNWTSGFNKCA
VGAACQPFHFYFPTPTVLCNEIWTHSYKVSNYSRGS
GRCIQMWFDPAQGNPNEEVARFYAAAMS.

Embodiment 33. The system of any one of Embodiments 1 to 30, wherein the targeting adaptor shares at least 95% identity to:

(SEQ ID NO: 60)
HHWRYGGDPPWPRVSPACAGRFQSPVDIRPQLAAFS
PALRPLELLGFQLPPLPELRLRNNGHSVQLTLPPGL
EMALGPGREYRALQLHLHWGAAGRPGSEHTVEGHRF
PAEIHVVHLSTAFARVDEALGRPGGLAVLAAFLEEG
PEENSA YEQLLSRLEEIAEEGSETQVPGLDISALL
PSDFSRYFQYEGSLTTPPCAQGVIWTVFNQTVMLSA
KQLHTLSDTLWGPGDSRLQLNFRATQPLNGRVIEAS
FPAGVDESKYGPPCPPCPGGRMALIVLGGVAGLLLF
IGLGIFFCVRCRHRRRQ.

Embodiment 34. The system of any one of Embodiments 1 to 30, wherein the targeting adaptor shares at least 95% identity to:

(SEQ ID NO: 61)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCP.

Embodiment 35. The system of any one of Embodiments 1 to 34, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gp120 or a fragment thereof.
Embodiment 36. The system of Embodiment 35, wherein the cell-surface antigen is CD19.
Embodiment 37. The system of Embodiment 36, wherein the tLBD-1 shares at least 95% identity to:

(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVS
S.

Embodiment 38. A method of treating a disease or disorder in a subject in need thereof, comprising administering the system of any one of Embodiments 1 to 37 to the subject.
Embodiment 39. The method of Embodiment 38, comprising administering the engineered immune cell comprising the chimeric receptor to the subject.
Embodiment 40. The method of Embodiment 38, comprising administering a vector encoding the chimeric receptor to the subject.
Embodiment 41. The method of any one of Embodiments 38 to 40, comprising administering the targeting adaptor to the subject.
Embodiment 42. The method of any one of Embodiments 38 to 40, comprising administering a vector encoding the targeting adaptor to the subject.
Embodiment 43. The method of any one of Embodiments 38 to 42, comprising administering the gating adaptor to the subject.
Embodiment 44. The method of Embodiment 43, comprising withholding the gating adaptor from the subject when a side effect of treatment is observed.
Embodiment 45. A method of generating a Gated Adaptor Targeting Receptor (GATR) system and/or treating a disease or disorder in a subject in need thereof, comprising:
(a) administering to the subject an engineered immune cell comprising a chimeric receptor, optionally a chimeric antigen receptor, or a vector encoding the chimeric receptor;
(b) administering to the subject a targeting adaptor, or vector encoding the targeting adaptor;
(c) administering to the subject a gating adaptor;
wherein the targeting adaptor comprises a first ligand-binding domain (tLBD-1) specific for a cell-surface antigen and a second ligand-binding domain (tLBD-2) specific for the gating adaptor; and
wherein the chimeric receptor comprises an extracellular ligand-binding domain (rLBD) specific for the gating adaptor, a transmembrane domain, and an intracellular actuator domain.
wherein the method generates an effective amount of the GATR system in the subject.
Embodiment 46. The method of Embodiment 45, wherein the rLBD comprises an antibody or antigen-binding fragment thereof.
Embodiment 47. The method of Embodiment 46, wherein the rLBD comprises a single-chain variable fragment (scFv).
Embodiment 48. The method of Embodiment 45, wherein the rLBD comprises a T-cell receptor (TCR) or antigen-binding fragment thereof.
Embodiment 49. The method of any one of Embodiments 45 to 48, wherein the tLBD-1 comprises an antibody or antigen-binding fragment thereof.
Embodiment 50. The method of Embodiment 49, wherein the tLBD-1 comprises a single-chain variable fragment (scFv).
Embodiment 51. The method of any one of Embodiments 45 to 48, wherein the tLBD-1 comprises a T-cell receptor (TCR) or antigen-binding fragment thereof.
Embodiment 52. The method of any one of Embodiments 45 to 51, wherein the tLBD-2 comprises an antibody or antigen-binding fragment thereof.
Embodiment 53. The method of any one of Embodiments 45 to 51, wherein the tLBD-2 comprises a folate receptor domain.
Embodiment 54. The method of Embodiment 53, wherein the folate receptor domain is a folate receptor alpha (FRα) domain.
Embodiment 55. The method of Embodiment 54, wherein the FRα domain shares at least 95% identity to:

(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

Embodiment 56. The method of any one of Embodiments 45 to 51, wherein the tLBD-2 comprises a carbonic anhydrase IX (CA9) domain.
Embodiment 57. The method of Embodiment 56, wherein the CA9 domain shares at least 95% identity to:

(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

Embodiment 58. The method of Embodiment 56, wherein the CA9 domain shares at least 95% identity to:

(SEQ ID NO: 20)
HWRYGGDPPWPRVSPACAGRFQSPVDIRPQLAAFS
PALRPLELSGFQLPPLPELRLRNNGHSVQLTLPPG
LEMKLGPGREYRALQLHLHWGAAGRPGSEHTVEGH
RFPAEIHVVHLSTKYARVDEALGRPGGLAVLAAFL
EEGPEENSAYEQLLSRLEEIAEEGSETQVPGLDIS
ALLPSDFSRYFQYEGSLTTPPCAQGVIWTVFNQTV
SLSAKQLHTLSDTLWGPGDSRLQLNFRATQPLNGR
VIEASFPAGVD.

Embodiment 59. The method of any one of Embodiments 45 to 58, wherein the gating adaptor is a small molecule.
Embodiment 60. The method of Embodiment 59, wherein the gating adaptor comprises a first moiety recognized by rLBD and a second moiety recognized by tLBD-2.
Embodiment 61. The method of Embodiment 60, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 62. The method of Embodiment 61, wherein the first moiety is fluorescein or a derivative thereof.
Embodiment 63. The method of any one of Embodiments 60 to 62, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 64. The method of Embodiment 63, wherein the second moiety is folate or a derivative thereof.
Embodiment 65. The method of Embodiment 63, wherein the second moiety is a CA9 ligand or a derivative thereof.
Embodiment 66. The method of Embodiment 60, wherein the gating adaptor is a folate-fluorescein conjugate.
Embodiment 67. The method of Embodiment 60, wherein the gating adaptor is a CA9 ligand-fluorescein conjugate.
Embodiment 68. The method of any one of Embodiments 45 to 67, wherein the chimeric receptor comprises one polypeptide chain.
Embodiment 69. The method of any one of Embodiments 45 to 67, wherein the chimeric receptor comprises at least two polypeptide chains.
Embodiment 70. The method of any one of Embodiments 45 to 69, wherein the chimeric receptor specifically binds fluorescein.
Embodiment 71. The method of Embodiment 70, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.
Embodiment 72. The method of Embodiment 70, wherein the rLBD comprises a polypeptide sequence sharing at least 95% identity to:

(SEQ ID NO: 31)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLG;
and
a polypeptide sequence sharing
at least 95% identity to:
(SEQ ID NO: 32)
QVQLVESGGNLVQPGGSLRLSCAASGFTFGSFSMS
WVRQAPGGGLEWVAGLSARSSLTHYADSVKGRFTI
SRDNAKNSVYLQMNSLRVEDTAVYYCARRSYDSSG
YWGHFYSYMDVWGQGTLVTVSS.

Embodiment 73. The method of Embodiment 72, wherein the rLBD shares at least 95% identity to:

(SEQ ID NO: 30)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSS.

Embodiment 74. The method of Embodiment 73, wherein the chimeric receptor shares at least 95% identity to:

(SEQ ID NO: 58)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSSESKYGPPCPPCPMFWVLVVVGGVLAC
YSLLVTVAFIIFWVKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPR.

Embodiment 75. The method of any one of Embodiments 45 to 74, wherein the targeting adaptor shares at least 95% identity to:

(SEQ ID NO: 59)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMSES
KYGPPCPPCPGGRMALIVLGGVAGLLLFIGLGIFF
CVRCRHRRRQ.

Embodiment 76. The method of any one of Embodiments 45 to 74, wherein the targeting adaptor shares at least 95% identity to:

(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

Embodiment 77. The method of any one of Embodiments 45 to 74, wherein the targeting adaptor shares at least 95% identity to:

(SEQ ID NO: 60)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCPGGRMALIVLGG
VAGLLLFIGLGIFFCVRCRHRRRQ.

Embodiment 78. The method of any one of Embodiments 45 to 74, wherein the targeting adaptor shares at least 95% identity to:

(SEQ ID NO: 61)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCP.

Embodiment 79. The method of any one of Embodiments 45 to 78, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen, CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gpl20 or a fragment thereof.
Embodiment 80. The method of Embodiment 79, wherein the cell-surface antigen is CD19.
Embodiment 81. The method of Embodiment 80, wherein the tLBD-1 shares at least 95% identity to:

(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVSS.

Embodiment 82. A targeting adaptor, comprising a first ligand-binding domain (tLBD-1) specific for a cell-surface antigen and a second ligand-binding domain (tLBD-2) specific for a moiety select from folate, a CA9 ligand, fluorescein, and a derivative thereof.
Embodiment 83. The targeting adaptor of Embodiment 82, wherein the tLBD-1 comprises an antibody or antigen-binding fragment thereof, optionally a single-chain variable fragment (scFv).
Embodiment 84. The targeting adaptor of Embodiment 82, wherein the tLBD-2 comprises an antibody or antigen-binding fragment thereof, optionally a single-chain variable fragment (scFv).
Embodiment 85. The targeting adaptor of any one of Embodiments 82 to 84, wherein the tLBD-2 comprises a folate receptor domain.
Embodiment 86. The targeting adaptor of Embodiment 85, wherein the folate receptor domain is a folate receptor alpha (FRα) domain.
Embodiment 87. The targeting adaptor of Embodiment 86, wherein the FRα domain shares at least 95% identity to:

(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

Embodiment 88. The targeting adaptor of any one of Embodiments 82 to 84, wherein the tLBD-2 comprises a carbonic anhydrase IX (CA9) domain.
Embodiment 89. The targeting adaptor of Embodiment 88, wherein the CA9 domain shares at least 95% identity to:

(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

Embodiment 90. The targeting adaptor of Embodiment 88, wherein the CA9 domain shares at least 95% identity to:

(SEQ ID NO: 20)
HWRYGGDPPWPRVSPACAGRFQSPVDIRPQLAAFS
PALRPLELSGFQLPPLPELRLRNNGHSVQLTLPPG
LEMKLGPGREYRALQLHLHWGAAGRPGSEHTVEGH
RFPAEIHVVHLSTKYARVDEALGRPGGLAVLAAFL
EEGPEENSAYEQLLSRLEEIAEEGSETQVPGLDIS
ALLPSDFSRYFQYEGSLTTPPCAQGVIWTVFNQTV
SLSAKQLHTLSDTLWGPGDSRLQLNFRATQPLNGR
VIEASFPAGVD.

Embodiment 91. A polynucleotide encoding the targeting adaptor of any one of Embodiments 82 to 90.
Embodiment 92. A vector comprising the polynucleotide of Embodiment 91.
Embodiment 93. A kit comprising the system of any one of Embodiments 1 to 37 and instructions for use.
Embodiment 94. The kit of Embodiment 93, comprising the engineered immune cell comprising the chimeric receptor.
Embodiment 95. The kit of Embodiment 93, comprising the vector encoding the chimeric receptor.
Embodiment 96. A vector for use with a gating adaptor, comprising:
(a) a polynucleotide encoding a targeting adaptor; and
(b) a polynucleotide encoding a chimeric receptor, optionally a chimeric antigen receptor,
wherein the targeting adaptor comprises a first ligand-binding domain (tLBD-1) specific for a cell-surface antigen and a second ligand-binding domain (tLBD-2) specific for the gating adaptor; and
wherein the chimeric receptor comprises an extracellular ligand-binding domain (rLBD) specific for the gating adaptor, a transmembrane domain, and an intracellular actuator domain.
Embodiment 97. The vector of Embodiment 96, wherein the vector is a viral vector.
Embodiment 98. The vector of Embodiment 97, wherein the viral vector is a retroviral vector.
Embodiment 99. The vector of Embodiment 98, wherein the retroviral vector is a lentiviral vector.
Embodiment 100. The vector of Embodiment 98, wherein the retroviral vector is a gamma-retroviral vector.
Embodiment 101. The vector of any one of Embodiments 97 to 100, wherein the viral vector comprises a VSV G protein or functional variant thereof.
Embodiment 102. The vector of any one of Embodiments 97 to 100, wherein the viral vector comprises a Cocal G protein or functional variant thereof.
Embodiment 103. The vector of Embodiment 96, wherein the rLBD comprises an antibody or antigen-binding fragment thereof.
Embodiment 104. The vector of Embodiment 103, wherein the rLBD comprises a single-chain variable fragment (scFv).
Embodiment 105. The vector of Embodiment 96, wherein the rLBD comprises a T-cell receptor (TCR) or antigen-binding fragment thereof.
Embodiment 106. The vector of any one of Embodiments 96 to 105, wherein the tLBD-1 comprises an antibody or antigen-binding fragment thereof.
Embodiment 107. The vector of Embodiment 106, wherein the tLBD-1 comprises a single-chain variable fragment (scFv).
Embodiment 108. The vector of any one of Embodiments 96 to 105, wherein the tLBD-1 comprises a T-cell receptor (TCR) or antigen-binding fragment thereof.
Embodiment 109. The vector of any one of Embodiments 96 to 108, wherein the tLBD-2 comprises an antibody or antigen-binding fragment thereof.
Embodiment 110. The vector of any one of Embodiments 96 to 108, wherein the tLBD-2 comprises a folate receptor domain.
Embodiment 111. The vector of Embodiment 110, wherein the folate receptor domain is a folate receptor alpha (FRα) domain.
Embodiment 112. The vector of Embodiment 111, wherein the FRα domain shares at least 95% identity to:

(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

Embodiment 113. The vector of any one of Embodiments 96 to 108, wherein the tLBD-2 comprises a carbonic anhydrase IX (CA9) domain.
Embodiment 114. The vector of Embodiment 113, wherein the CA9 domain shares at least 95% identity to:

(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

Embodiment 115. The vector of Embodiment 113, wherein the CA9 domain shares at least 95% identity to:

(SEQ ID NO: 20)
HWRYGGDPPWPRVSPACAGRFQSPVDIRPQLAAFS
PALRPLELSGFQLPPLPELRLRNNGHSVQLTLPPG
LEMKLGPGREYRALQLHLHWGAAGRPGSEHTVEGH
RFPAEIHVVHLSTKYARVDEALGRPGGLAVLAAFL
EEGPEENSAYEQLLSRLEEIAEEGSETQVPGLDIS
ALLPSDFSRYFQYEGSLTTPPCAQGVIWTVFNQTV
SLSAKQLHTLSDTLWGPGDSRLQLNFRATQPLNGR
VIEASFPAGVD.

Embodiment 116. The vector of any one of Embodiments 96 to 115, wherein the gating adaptor is a small molecule.
Embodiment 117. The vector of Embodiment 116, wherein the gating adaptor comprises a first moiety recognized by rLBD and a second moiety recognized by tLBD-2.
Embodiment 118. The vector of Embodiment 117, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 119. The vector of Embodiment 118, wherein the first moiety is fluorescein or a derivative thereof.
Embodiment 120. The vector of any one of Embodiments 117 to 119, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 121. The vector of Embodiment 120, wherein the second moiety is folate or a derivative thereof.
Embodiment 122. The vector of Embodiment 120, wherein the second moiety is a CA9 ligand or a derivative thereof.
Embodiment 123. The vector of Embodiment 121, wherein the gating adaptor is a folate-fluorescein conjugate.
Embodiment 124. The vector of Embodiment 120, wherein the gating adaptor is a CA9 ligand-fluorescein conjugate.
Embodiment 125. The vector of any one of Embodiments 96 to 124, wherein the chimeric receptor comprises one polypeptide chain.
Embodiment 126. The vector of any one of Embodiments 96 to 124, wherein the chimeric receptor comprises at least two polypeptide chains.
Embodiment 127. The vector of any one of Embodiments 96 to 126, wherein the chimeric receptor specifically binds fluorescein.
Embodiment 128. The vector of Embodiment 127, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.
Embodiment 129. The vector of Embodiment 127, wherein the rLBD comprises a polypeptide sequence sharing at least 95% identity to:

(SEQ ID NO: 31)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLG;

and a polypeptide sequence sharing at least 95% identity to:

(SEQ ID NO: 32)
QVQLVESGGNLVQPGGSLRLSCAASGFTFGSFSMS
WVRQAPGGGLEWVAGLSARSSLTHYADSVKGRFTI
SRDNAKNSVYLQMNSLRVEDTAVYYCARRSYDSSG
YWGHFYSYMDVWGQGTLVTVSS.

Embodiment 130. The vector of Embodiment 129, wherein the rLBD shares at least 95% identity to:

(SEQ ID NO: 30)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSS.

Embodiment 131. The vector of Embodiment 130, wherein the chimeric receptor shares at least 95% identity to:

(SEQ ID NO: 58)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSSESKYGPPCPPCPMFWVLVVVGGVLAC
YSLLVTVAFIIFWVKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPR.

Embodiment 132. The vector of any one of Embodiments 96 to 131, wherein the targeting adaptor shares at least 95% identity to:

(SEQ ID NO: 59)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMSES
KYGPPCPPCPGGRMALIVLGGVAGLLLFIGLGIFF
CVRCRHRRRQ.

Embodiment 133. The vector of any one of Embodiments 96 to 131, wherein the targeting adaptor shares at least 95% identity to:

(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

Embodiment 134. The vector of any one of Embodiments 96 to 131, wherein the targeting adaptor shares at least 95% identity to:

(SEQ ID NO: 60)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCPGGRMALIVLGG
VAGLLLFIGLGIFFCVRCRHRRRQ.

Embodiment 135. The vector of any one of Embodiments 96 to 131, wherein the targeting adaptor shares at least 95% identity to:

(SEQ ID NO: 61)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCP.

Embodiment 136. The vector of any one of Embodiments 96 to 135, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gp120 or a fragment thereof.
Embodiment 137. The vector of Embodiment 136, wherein the cell-surface antigen is CD19.
Embodiment 138. The vector of Embodiment 137, wherein the tLBD-1 shares at least 95% identity to:

(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVSS.

Embodiment 139. An isolated cell, comprising:
(a) a polynucleotide encoding a targeting adaptor, and/or a targeting adaptor comprising a transmembrane domain and expressed on the surface of the isolated cell; and
(b) a polynucleotide encoding a chimeric receptor, optionally a chimeric antigen receptor, and/or a chimeric receptor expressed on the surface of the isolated cell,
wherein the targeting adaptor comprises a first ligand-binding domain (tLBD-1) specific for a cell-surface antigen and a second ligand-binding domain (tLBD-2) specific for a gating adaptor; and
wherein the chimeric receptor comprises an extracellular ligand-binding domain (rLBD) specific for the gating adaptor, a transmembrane domain, and an intracellular actuator domain.
Embodiment 140. The cell of Embodiment 139, wherein the rLBD comprises an antibody or antigen-binding fragment thereof.
Embodiment 141. The cell of Embodiment 140, wherein the rLBD comprises a single-chain variable fragment (scFv).
Embodiment 142. The cell of Embodiment 139, wherein the rLBD comprises a T-cell receptor (TCR) or antigen-binding fragment thereof.
Embodiment 143. The cell of any one of Embodiments 139 to 142, wherein the tLBD-1 comprises an antibody or antigen-binding fragment thereof.
Embodiment 144. The cell of Embodiment 143, wherein the tLBD-1 comprises a single-chain variable fragment (scFv).
Embodiment 145. The cell of any one of Embodiments 139 to 142, wherein the tLBD-1 comprises a T-cell receptor (TCR) or antigen-binding fragment thereof.
Embodiment 146. The cell of any one of Embodiments 139 to 145, wherein the tLBD-2 comprises an antibody or antigen-binding fragment thereof.
Embodiment 147. The cell of any one of Embodiments 139 to 145, wherein the tLBD-2 comprises a folate receptor domain.
Embodiment 148. The cell of Embodiment 147, wherein the folate receptor domain is a folate receptor alpha (FRα) domain.
Embodiment 149. The cell of Embodiment 148, wherein the FRα domain shares at least 95% identity to:

(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

Embodiment 150. The cell of any one of Embodiments 139 to 145, wherein the tLBD-2 comprises a carbonic anhydrase IX (CA9) domain.
Embodiment 151. The cell of Embodiment 150, wherein the CA9 domain shares at least 95% identity to:

(SEQ ID NO: 20)
HWRYGGDPPWPRVSPACAGRFQSPVDIRPQLAAFSPALRPLELSGFQLP
PLPELRLRNNGHSVQLTLPPGLEMKLGPGREYRALQLHLHWGAAGRPGS
EHTVEGHRFPAEIHVVHLSTKYARVDEALGRPGGLAVLAAFLEEGPEEN
SAYEQLLSRLEEIAEEGSETQVPGLDISALLPSDFSRYFQYEGSLTTPP
CAQGVIWTVFNQTVSLSAKQLHTLSDTLWGPGDSRLQLNFRATQPLNGR
VIEASFPAGVD.

Embodiment 152. The cell of Embodiment 150, wherein the CA9 domain shares at least 95% identity to:

(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

Embodiment 153. The cell of any one of Embodiments 139 to 152, wherein the gating adaptor is a small molecule.
Embodiment 154. The cell of Embodiment 153, wherein the gating adaptor comprises a first moiety recognized by rLBD and a second moiety recognized by tLBD-2.
Embodiment 155. The cell of Embodiment 154, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 156. The cell of Embodiment 155, wherein the first moiety is fluorescein or a derivative thereof.
Embodiment 157. The cell of any one of Embodiments 154 to 156, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.
Embodiment 158. The cell of Embodiment 157, wherein the second moiety is folate or a derivative thereof.
Embodiment 159. The cell of Embodiment 157, wherein the second moiety is a CA9 ligand or a derivative thereof.
Embodiment 160. The cell of Embodiment 158, wherein the gating adaptor is a folate-fluorescein conjugate.
Embodiment 161. The cell of Embodiment 157, wherein the gating adaptor is a CA9 ligand-fluorescein conjugate.
Embodiment 162. The cell of any one of Embodiments 139 to 161, wherein the chimeric receptor comprises one polypeptide chain.
Embodiment 163. The cell of any one of Embodiments 139 to 161, wherein the chimeric receptor comprises at least two polypeptide chains.
Embodiment 164. The cell of any one of Embodiments 139 to 163, wherein the chimeric receptor specifically binds fluorescein.
Embodiment 165. The cell of Embodiment 164, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.
Embodiment 166. The cell of Embodiment 164, wherein the rLBD comprises a polypeptide sequence sharing at least 95% identity to:

(SEQ ID NO: 31)
SVLTQPSSVSAAPGQKVTISCSGSTSNIGNNYVSWYQQHPGKAPKLMIY
DVSKRPSGVPDRFSGSKSGNSASLDISGLQSEDEADYYCAAWDDSLSEF
LFGTGTKLTVLG;

and a polypeptide sequence sharing at least 95% identity to:

(SEQ ID NO: 32)
QVQLVESGGNLVQPGGSLRLSCAASGFTFGSFSMSWVRQAPGGGLEWVA
GLSARSSLTHYADSVKGRFTISRDNAKNSVYLQMNSLRVEDTAVYYCAR
RSYDSSGYWGHFYSYMDVWGQGTLVTVSS.

Embodiment 167. The cell of Embodiment 166, wherein the rLBD shares at least 95% identity to:

(SEQ ID NO: 30)
SVLTQPSSVSAAPGQKVTISCSGSTSNIGNNYVSWYQQHPGKAPKLMIY
DVSKRPSGVPDRFSGSKSGNSASLDISGLQSEDEADYYCAAWDDSLSEF
LFGTGTKLTVLGSTSGSGKPGSGEGSTKGQVQLVESGGNLVQPGGSLRL
SCAASGFTFGSFSMSWVRQAPGGGLEWVAGLSARSSLTHYADSVKGRFT
ISRDNAKNSVYLQMNSLRVEDTAVYYCARRSYDSSGYWGHFYSYMDVWG
QGTLVTVSS.

Embodiment 168. The cell of Embodiment 167, wherein the chimeric receptor shares at least 95% identity to:

(SEQ ID NO: 58)
SVLTQPSSVSAAPGQKVTISCSGSTSNIGNNYVSWYQQHPGKAPKLMIY
DVSKRPSGVPDRFSGSKSGNSASLDISGLQSEDEADYYCAAWDDSLSEF
LFGTGTKLTVLGSTSGSGKPGSGEGSTKGQVQLVESGGNLVQPGGSLRL
SCAASGFTFGSFSMSWVRQAPGGGLEWVAGLSARSSLTHYADSVKGRFT
ISRDNAKNSVYLQMNSLRVEDTAVYYCARRSYDSSGYWGHFYSYMDVWG
QGTLVTVSSESKYGPPCPPCPMFWVLVVVGGVLACYSLLVTVAFIIFWV
KRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALH
MQALPPR.

Embodiment 169. The cell of any one of Embodiments 139 to 168, wherein the targeting adaptor shares at least 95% identity to:

(SEQ ID NO: 59)
GSSRTELLNVCMNAKHHKEKPGPEDKLHEQCRPWRKNACCSTNTSQEAH
KDVSYLYRFNWNHCGEMAPACKRHFIQDTCLYECSPNLGPWIQQVDQSW
RKERVLNVPLCKEDCEQWWEDCRTSYTCKSNWHKGWNWTSGFNKCAVGA
ACQPFHFYFPTPTVLCNEIWTHSYKVSNYSRGSGRCIQMWFDPAQGNPN
EEVARFYAAAMSESKYGPPCPPCPGGRMALIVLGGVAGLLLFIGLGIFF
CVRCRHRRRQ.

Embodiment 170. The cell of any one of Embodiments 139 to 168, wherein the targeting adaptor shares at least 95% identity to:

(SEQ ID NO: 18)
GSSRTELLNVCMNAKHHKEKPGPEDKLHEQCRPWRKNACCSTNTSQEAH
KDVSYLYRFNWNHCGEMAPACKRHFIQDTCLYECSPNLGPWIQQVDQSW
RKERVLNVPLCKEDCEQWWEDCRTSYTCKSNWHKGWNWTSGFNKCAVGA
ACQPFHFYFPTPTVLCNEIWTHSYKVSNYSRGSGRCIQMWFDPAQGNPN
EEVARFYAAAMS.

Embodiment 171. The cell of any one of Embodiments 139 to 168, wherein the targeting adaptor shares at least 95% identity to:

(SEQ ID NO: 60)
HHWRYGGDPPWPRVSPACAGRFQSPVDIRPQLAAFSPALRPLELLGFQL
PPLPELRLRNNGHSVQLTLPPGLEMALGPGREYRALQLHLHWGAAGRPG
SEHTVEGHRFPAEIHVVHLSTAFARVDEALGRPGGLAVLAAFLEEGPEE
NSAYEQLLSRLEEIAEEGSETQVPGLDISALLPSDFSRYFQYEGSLTTP
PCAQGVIWTVFNQTVMLSAKQLHTLSDTLWGPGDSRLQLNFRATQPLNG
RVIEASFPAGVDESKYGPPCPPCPGGRMALIVLGGVAGLLLFIGLGIFF
CVRCRHRRRQ.

Embodiment 172. The cell of any one of Embodiments 139 to 168, wherein the targeting adaptor shares at least 95% identity to:

(SEQ ID NO: 61)
HHWRYGGDPPWPRVSPACAGRFQSPVDIRPQLAAFSPALRPLELLGFQL
PPLPELRLRNNGHSVQLTLPPGLEMALGPGREYRALQLHLHWGAAGRPG
SEHTVEGHRFPAEIHVVHLSTAFARVDEALGRPGGLAVLAAFLEEGPEE
NSAYEQLLSRLEEIAEEGSETQVPGLDISALLPSDFSRYFQYEGSLTTP
PCAQGVIWTVFNQTVMLSAKQLHTLSDTLWGPGDSRLQLNFRATQPLNG
RVIEASFPAGVDESKYGPPCPPCP.

Embodiment 173. The cell of any one of Embodiments 139 to 172, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gpl20 or a fragment thereof.
Embodiment 174. The cell of Embodiment 173, wherein the cell-surface antigen is CD19.
Embodiment 175. The cell of Embodiment 174, wherein the tLBD-1 shares at least 95% identity to:

(SEQ ID NO: 52)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGSTSGSGKPGSGEGSTKGEVKLQESGPGLVAPSQSLSVTC
TVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSS.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what is regarding as the invention.

### Example 1: GATR Cell Transduction

This example demonstrates expression of a GATR system in primary human T cells and drug-inducible cytokine production in co-culture with CD19+ target cells. FITC-Aza is used as the gating adaptor.

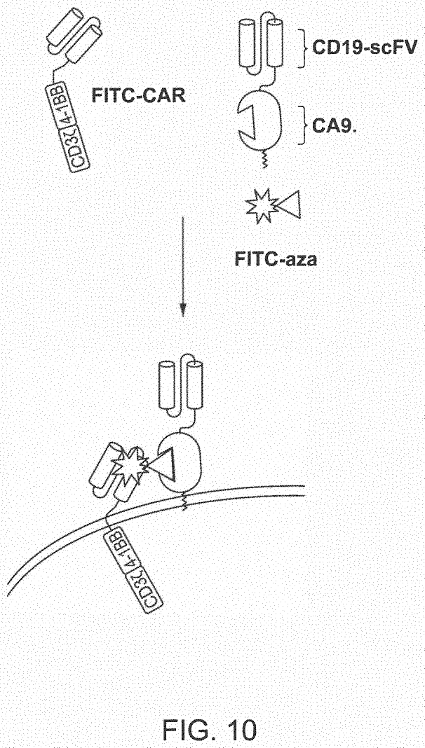

Primary CD3+ T-cells (˜15 million cells, Bloodworks donor 3251BW) were thawed, rescued, and bead-stimulated (1:1) for 48 hours. The stimulated T-cells were transduced with a lentiviral vector encoding two membrane proteins: a receptor in which an anti-fluorescein scFv is fused via a transmembrane domain to a 41bb-zeta intracellular domain; and a receptor in which an anti-CD19 scFv is fused to a CA9 domain which is in turn fused to a transmembrane domain. As shown in FIG. 10, the system is designed to permit a fluorescein-acetazolamide (“FITC-Aza”) to gate binding of the two proteins, inducibly generating an anti-CD19 CAR. Transduction was performed at with serially diluted vector at multiplicity of infection (MOI) of 0.0325, 0.1625, 0.325, or 1.625.

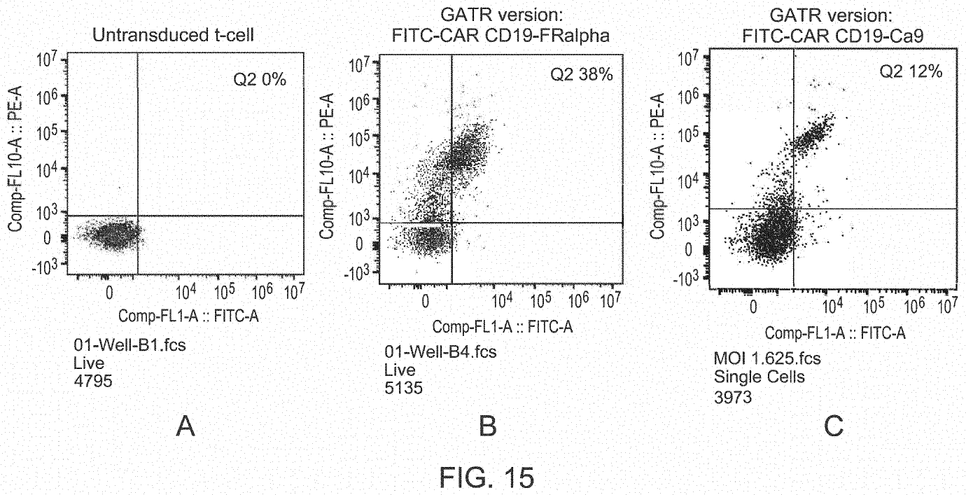

Four days after transduction, the T cells were then stained with FITC-Dextran and analyzed by flow cytometry to measure GATR FITC-CAR expression (FIGS. 15A and 15C). As compared to untransduced T-cells (0%), cells transduced with the GATR FITC-CAR CD19-CA9 demonstrate GATR surface expression (12%).

### Staining Procedure

The following antibodies were used in flow cytometry analysis:
a. FITC-DEX—3 μl/50 μl in sample, 1 μg/ml (Sigma Millipore)
b. CD19 (aa 20-291)PE conjugate—2 μl/50 μl (stock diluted 1/100)
c. anti-CD8 AF700 (Biolegend)—1 μl/50 μl
d. anti-CD4 BV650 (Biolegend)—1 μl/50 μl
e. DAPI 1 μl/100 μl

Staining procedure: Cells were spun down in 96 well plate, washed once in 250 μl PBS and spun for 5′ at 1400 RPM (300 g). Cells were suspended in 50 μl MACS/1% BSA (Beckman Coulter) with staining reagents as above. Cells were then incubated for 40 min at RT in the dark, pseudo-washed in 150 μl PBS, spun, re-suspended in 1 μl 100×DAPI in 100 μl MACS, incubated for 10 min at RT in the dark, spun, washed with 250 μl PBS and re-suspended 100 μl CytoFix (Biolegend). Flow Cytometry analysis was performed using Cytoflex-Beckman Coulter-Blue, Violet, Red, Yellow.

The T-cell transduction efficiency at variable MOI is described in Table 1.

TABLE 1

| | Percentage of FITC- |
|---|---|
| MOI | Dextran + cells |
| 0 | 1.00 |
| 0.0325 | 0.72 |
| 0.1625 | 4.94 |
| 0.325 | 8.82 |
| 1.625 | 11.5 |

Transduced cells were co-cultured with CD19+K562 target cells or CD19-K562 control cells in 250 μl of media at a 4:1 ratio. FITC-Aza was added at a concentration of 1 nM, 10 nM, 100 nM, 1,000 nM, or 10,000 nM. The cells were co-cultured in the presence of FITC-Aza for 72 hours.

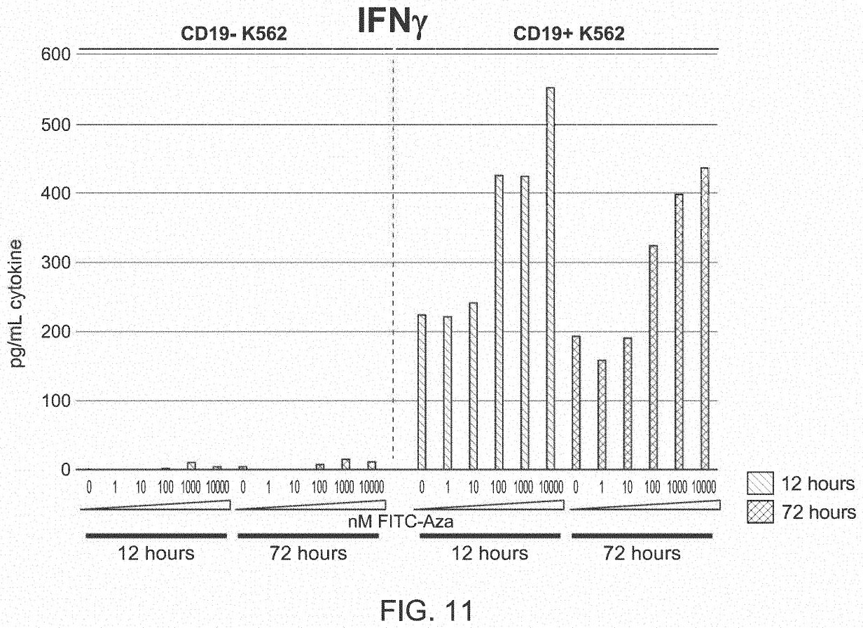

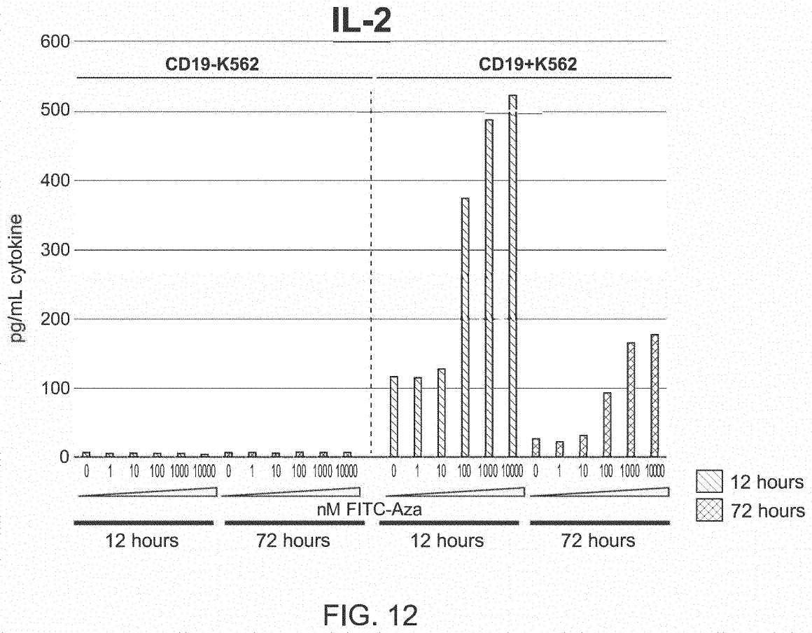

Cytokine analysis was performed for INFγ (FIG. 11) and IL-2 (FIG. 12). Cytokine production increased in a dose-dependent manner with the increase in concentration of the gating molecule FITC-Aza. Transduced cells did not produce cytokines in the presence of control CD19-K562 cells.

### Example 2: GATR Design and Cell Transduction

This example demonstrates expression of a GATR system comprising a an anti-fluorescein scFv is fused via a transmembrane domain to a 41bb-zeta intracellular domain; and a targeting receptor in which an anti-CD19 scFv is fused to a FRalpha domain which in turn is fused to a transmembrane domain. FITC-folate is used as the gating adaptor.

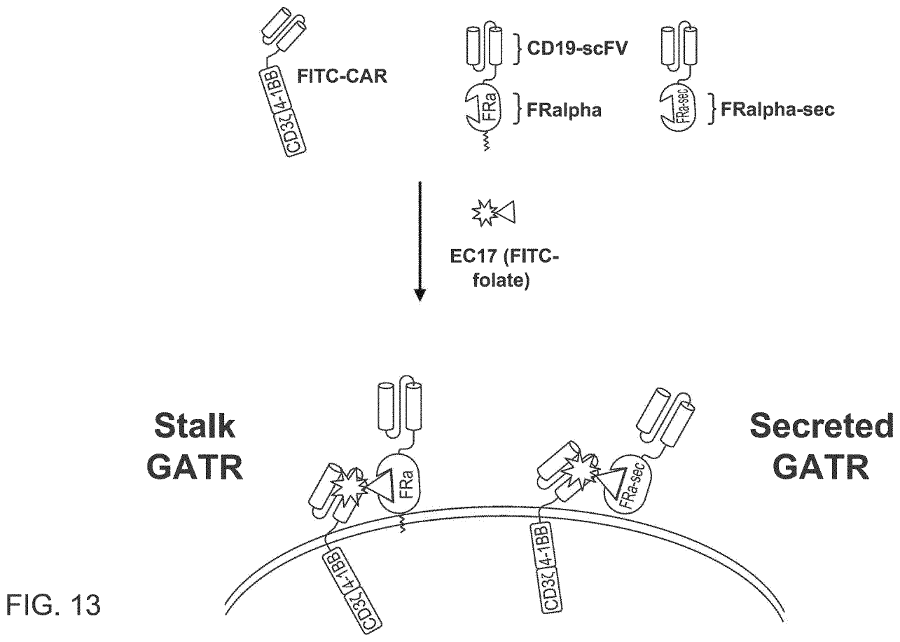

Primary CD3+ T-cells (Bloodworks donor 3251BW) were thawed, rescued, and bead-stimulated (1:1) for 48 hours. The stimulated T-cells were transduced with a lentiviral vector encoding two membrane proteins: a receptor in which an anti-fluorescein scFv is fused via a transmembrane domain to a 41bb-zeta intracellular domain; and a receptor in which an anti-CD19 scFv is fused to a FRalpha domain which is in turn fused to a transmembrane domain. As shown in FIG. 13, the system is designed to permit a fluorescein-folate (“FITC-folate”) to gate binding of the two proteins, thereby inducibly bridging the receptor subunits to form a receptor dimer. T-cells were transduced with either an anti-CD19 FRalpha fusion peptide that has a stalk transmembrane domain, or an anti-CD19 FRalpha fusion peptide that lacks a stalk.

Four days following transduction, the T-cells were stained with FITC-Dextran and/or CD19-R-phycoerythrin (PE) and analyzed by flow cytometry to measure GATR FITC-CAR CD19-FRalpha expression (FIGS. 15A and 15B). As compared to untransduced T-cells (0%), cells transduced with the GATR FITC-CAR CD19-FRalpha demonstrate GATR surface expression (38%). The staining procedure and flow cytometry analysis was performed as described above.

Transduced cells were co-cultured with Raji target cells at a 1:1 ratio for 12 hours in media containing folate. EC17 (FITC-folate) was added at a concentration of 0.1, 1 nM, 10 nM, 100 nM, or 1,000 nM.

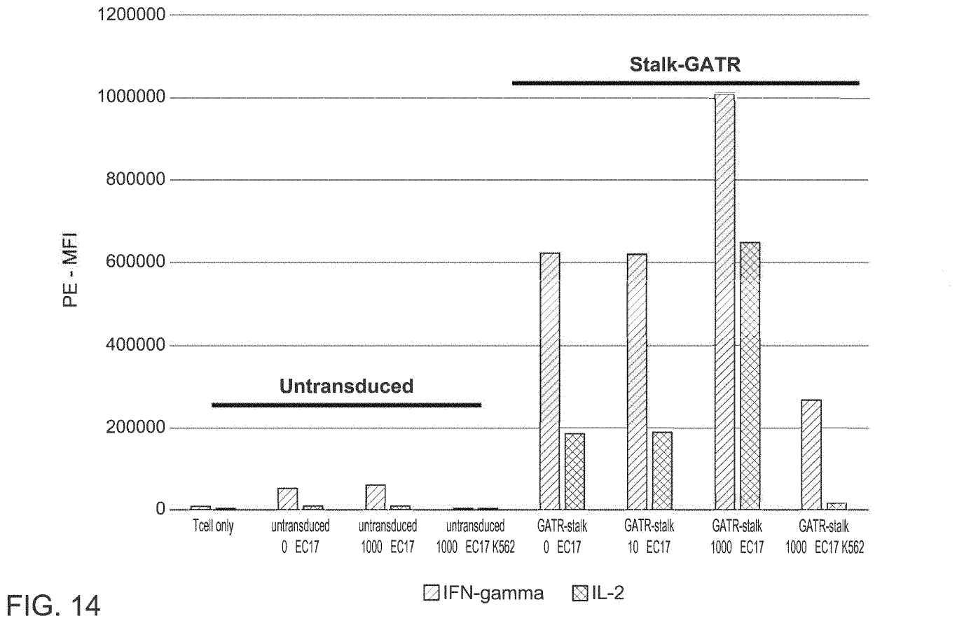

Cytokine analysis was performed for INFγ and IL-2 (FIG. 14). Cytokine production increased in a dose-dependent manner with the increase in concentration of the gating molecule EC17. Transduced cells did not produce cytokines in the presence of control K562 cells.

1. A Gated Adaptor Targeting Receptor (GATR) system, comprising:
(a) a gating adaptor;
(b) a targeting adaptor, or a vector encoding the targeting adaptor; and
(c) an engineered immune cell comprising a chimeric receptor, optionally a chimeric antigen receptor, or a vector encoding the chimeric receptor,
wherein the targeting adaptor comprises a first ligand-binding domain (tLBD-1) specific for a cell-surface antigen and a second ligand-binding domain (tLBD-2) specific for the gating adaptor; and
wherein the chimeric receptor comprises an extracellular ligand-binding domain (rLBD) specific for the gating adaptor, a transmembrane domain, and an intracellular actuator domain.

2. The system of claim 1, wherein the rLBD comprises an antibody or antigen-binding fragment thereof.

3. The system of claim 2, wherein the rLBD comprises a single-chain variable fragment (scFv).

4. The system of claim 1, wherein the rLBD comprises a T-cell receptor (TCR) or antigen-binding fragment thereof.

5. The system of any one of claims 1 to 4, wherein the tLBD-1 comprises an antibody or antigen-binding fragment thereof.

6. The system of claim 5, wherein the tLBD-1 comprises a single-chain variable fragment (scFv).

7. The system of any one of claims 1 to 4, wherein the tLBD-1 comprises a T-cell receptor (TCR) or antigen-binding fragment thereof.

8. The system of any one of claims 1 to 7, wherein the tLBD-2 comprises an antibody or antigen-binding fragment thereof.

9. The system of any one of claims 1 to 7, wherein the tLBD-2 comprises a folate receptor domain.

10. The system of claim 9, wherein the folate receptor domain is a folate receptor alpha (FRα) domain.

11. The system of claim 10, wherein the FRα domain shares at least 95% identity to:
(SEQ ID NO: 18)
GSSRTELLNVCMNAKHHKEKPGPEDKLHEQCRPWRKNACCSTNTSQEAH
KDVSYLYRFNWNHCGEMAPACKRHFIQDTCLYECSPNLGPWIQQVDQSW
RKERVLNVPLCKEDCEQWWEDCRTSYTCKSNWHKGWNWTSGFNKCAVGA
ACQPFHFYFPTPTVLCNEIWTHSYKVSNYSRGSGRCIQMWFDPAQGNPN
EEVARFYAAAMS.

12. The system of any one of claims 1 to 7, wherein the tLBD-2 comprises a carbonic anhydrase IX (CA9) domain.

13. The system of claim 12, wherein the CA9 domain shares at least 95% identity to:
(SEQ ID NO: 19)
HHWRYGGDPPWPRVSPACAGRFQSPVDIRPQLAAFSPALRPLELLGFQL
PPLPELRLRNNGHSVQLTLPPGLEMALGPGREYRALQLHLHWGAAGRPG
SEHTVEGHRFPAEIHVVHLSTAFARVDEALGRPGGLAVLAAFLEEGPEE
NSAYEQLLSRLEEIAEEGSETQVPGLDISALLPSDFSRYFQYEGSLTTP
PCAQGVIWTVFNQTVMLSAKQLHTLSDTLWGPGDSRLQLNFRATQPLNG
RVIEASFPAGVD.

14. The system of claim 12, wherein the CA9 domain shares at least 95% identity to:
(SEQ ID NO: 20)
HWRYGGDPPWPRVSPACAGRFQSPVDIRPQLAAFSPALRPLELSGFQLP
PLPELRLRNNGHSVQLTLPPGLEMKLGPGREYRALQLHLHWGAAGRPGS
EHTVEGHRFPAEIHVVHLSTKYARVDEALGRPGGLAVLAAFLEEGPEEN
SAYEQLLSRLEEIAEEGSETQVPGLDISALLPSDFSRYFQYEGSLTTPP
CAQGVIWTVFNQTVSLSAKQLHTLSDTLWGPGDSRLQLNFRATQPLNGR
VIEASFPAGVD.

15. The system of any one of claims 1 to 14, wherein the gating adaptor is a small molecule.

16. The system of claim 15, wherein the gating adaptor comprises a first moiety recognized by rLBD and a second moiety recognized by tLBD-2.

17. The system of claim 16, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.

18. The system of claim 17, wherein the first moiety is fluorescein or a derivative thereof.

19. The system of any one of claims 16 to 18, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.

20. The system of claim 19, wherein the second moiety is folate or a derivative thereof.

21. The system of claim 19, wherein the second moiety is a CA9 ligand or a derivative thereof.

22. The system of claim 20, wherein the gating adaptor is a folate-fluorescein conjugate.

23. The system of claim 19, wherein the gating adaptor is a CA9 ligand-fluorescein conjugate.

24. The system of any one of claims 1 to 23, wherein the chimeric receptor comprises one polypeptide chain.

25. The system of any one of claims 1 to 23, wherein the chimeric receptor comprises at least two polypeptide chains.

26. The system of any one of claims 1 to 25, wherein the chimeric receptor specifically binds fluorescein.

27. The system of claim 26, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.

28. The system of claim 26, wherein the rLBD comprises a polypeptide sequence sharing at least 95% identity to:
(SEQ ID NO: 31)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLG;
and a polypeptide sequence sharing at least 95% identity to:
(SEQ ID NO: 32)
QVQLVESGGNLVQPGGSLRLSCAASGFTFGSFSMSWVRQAPGGGLEWVA
GLSARSSLTHYADSVKGRFTISRDNAKNSVYLQMNSLRVEDTAVYYCAR
RSYDSSGYWGHFYSYMDVWGQGTLVTVSS.

29. The system of claim 28, wherein the rLBD shares at least 95% identity to:
(SEQ ID NO: 30)
SVLTQPSSVSAAPGQKVTISCSGSTSNIGNNYVSWYQQHPGKAPKLMIY
DVSKRPSGVPDRFSGSKSGNSASLDISGLQSEDEADYYCAAWDDSLSEF
LFGTGTKLTVLGSTSGSGKPGSGEGSTKGQVQLVESGGNLVQPGGSLRL
SCAASGFTFGSFSMSWVRQAPGGGLEWVAGLSARSSLTHYADSVKGRFT
ISRDNAKNSVYLQMNSLRVEDTAVYYCARRSYDSSGYWGHFYSYMDVWG
QGTLVTVSS.

30. The system of claim 29, wherein the chimeric receptor shares at least 95% identity to:
(SEQ ID NO: 58)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSSESKYGPPCPPCPMFWVLVVVGGVLAC
YSLLVTVAFIIFWVKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPR.

31. The system of any one of claims 1 to 30, wherein the targeting adaptor shares at least 95% identity to:
(SEQ ID NO: 59)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMSES
KYGPPCPPCPGGRMALIVLGGVAGLLLFIGLGIFF
CVRCRHRRRQ.

32. The system of any one of claims 1 to 30, wherein the targeting adaptor shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

33. The system of any one of claims 1 to 30, wherein the targeting adaptor shares at least 95% identity to:
(SEQ ID NO: 60)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCPGGRMALIVLGG
VAGLLLFIGLGIFFCVRCRHRRRQ.

34. The system of any one of claims 1 to 30, wherein the targeting adaptor shares at least 95% identity to:
(SEQ ID NO: 61)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCP.

35. The system of any one of claims 1 to 34, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gp120 or a fragment thereof.

36. The system of claim 35, wherein the cell-surface antigen is CD19.

37. The system of claim 36, wherein the tLBD-1 shares at least 95% identity to:
(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVSS.

38. A method of treating a disease or disorder in a subject in need thereof, comprising administering the system of any one of claims 1 to 37 to the subject.

39. The method of claim 38, comprising administering the engineered immune cell comprising the chimeric receptor to the subject.

40. The method of claim 38, comprising administering a vector encoding the chimeric receptor to the subject.

41. The method of any one of claims 38 to 40, comprising administering the targeting adaptor to the subject.

42. The method of any one of claims 38 to 40, comprising administering a vector encoding the targeting adaptor to the subject.

43. The method of any one of claims 38 to 42, comprising administering the gating adaptor to the subject.

44. The method of claim 43, comprising withholding the gating adaptor from the subject when a side effect of treatment is observed.

45. A method of generating a Gated Adaptor Targeting Receptor (GATR) system and/or treating a disease or disorder in a subject in need thereof, comprising:
(a) administering to the subject an engineered immune cell comprising a chimeric receptor, optionally a chimeric antigen receptor, or a vector encoding the chimeric receptor;
(b) administering to the subject a targeting adaptor, or vector encoding the targeting adaptor;
(c) administering to the subject a gating adaptor;
wherein the targeting adaptor comprises a first ligand-binding domain (tLBD-1) specific for a cell-surface antigen and a second ligand-binding domain (tLBD-2) specific for the gating adaptor; and
wherein the chimeric receptor comprises an extracellular ligand-binding domain (rLBD) specific for the gating adaptor, a transmembrane domain, and an intracellular actuator domain.
wherein the method generates an effective amount of the GATR system in the subject.

46. The method of claim 45, wherein the rLBD comprises an antibody or antigen-binding fragment thereof.

47. The method of claim 46, wherein the rLBD comprises a single-chain variable fragment (scFv).

48. The method of claim 45, wherein the rLBD comprises a T-cell receptor (TCR) or antigen-binding fragment thereof.

49. The method of any one of claims 45 to 48, wherein the tLBD-1 comprises an antibody or antigen-binding fragment thereof.

50. The method of claim 49, wherein the tLBD-1 comprises a single-chain variable fragment (scFv).

51. The method of any one of claims 45 to 48, wherein the tLBD-1 comprises a T-cell receptor (TCR) or antigen-binding fragment thereof.

52. The method of any one of claims 45 to 51, wherein the tLBD-2 comprises an antibody or antigen-binding fragment thereof.

53. The method of any one of claims 45 to 51, wherein the tLBD-2 comprises a folate receptor domain.

54. The method of claim 53, wherein the folate receptor domain is a folate receptor alpha (FRα) domain.

55. The method of claim 54, wherein the FRα domain shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

56. The method of any one of claims 45 to 51, wherein the tLBD-2 comprises a carbonic anhydrase IX (CA9) domain.

57. The method of claim 56, wherein the CA9 domain shares at least 95% identity to:
(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

58. The method of claim 56, wherein the CA9 domain shares at least 95% identity to:
(SEQ ID NO: 20)
HWRYGGDPPWPRVSPACAGRFQSPVDIRPQLAAFSP
ALRPLELSGFQLPPLPELRLRNNGHSVQLTLPPGLE
MKLGPGREYRALQLHLHWGAAGRPGSEHTVEGHRF
PAEIHVVHLSTKYARVDEALGRPGGLAVLAAFLEE
GPEENSAYEQLLSRLEEIAEEGSETQVPGLDISAL
LPSDFSRYFQYEGSLTTPPCAQGVIWTVFNQTVSL
SAKQLHTLSDTLWGPGDSRLQLNFRATQPLNGRVI
EASFPAGVD.

59. The method of any one of claims 45 to 58, wherein the gating adaptor is a small molecule.

60. The method of claim 59, wherein the gating adaptor comprises a first moiety recognized by rLBD and a second moiety recognized by tLBD-2.

61. The method of claim 60, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.

62. The method of claim 61, wherein the first moiety is fluorescein or a derivative thereof.

63. The method of any one of claims 60 to 62, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.

64. The method of claim 63, wherein the second moiety is folate or a derivative thereof.

65. The method of claim 63, wherein the second moiety is a CA9 ligand or a derivative thereof.

66. The method of claim 60, wherein the gating adaptor is a folate-fluorescein conjugate.

67. The method of claim 60, wherein the gating adaptor is a CA9 ligand-fluorescein conjugate.

68. The method of any one of claims 45 to 67, wherein the chimeric receptor comprises one polypeptide chain.

69. The method of any one of claims 45 to 67, wherein the chimeric receptor comprises at least two polypeptide chains.

70. The method of any one of claims 45 to 69, wherein the chimeric receptor specifically binds fluorescein.

71. The method of claim 70, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.

72. The method of claim 70, wherein the rLBD comprises a polypeptide sequence sharing at least 95% identity to:
(SEQ ID NO: 31)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLG;
and
a polypeptide sequence sharing
at least 95% identity to:
(SEQ ID NO: 32)
QVQLVESGGNLVQPGGSLRLSCAASGFTFGSFSMS
WVRQAPGGGLEWVAGLSARSSLTHYADSVKGRFTI
SRDNAKNSVYLQMNSLRVEDTAVYYCARRSYDSSG
YWGHFYSYMDVWGQGTLVTVSS.

73. The method of claim 72, wherein the rLBD shares at least 95% identity to:
(SEQ ID NO: 30)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSS.

74. The method of claim 73, wherein the chimeric receptor shares at least 95% identity to:
(SEQ ID NO: 58)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSSESKYGPPCPPCPMFWVLVVVGGVLAC
YSLLVTVAFIIFWVKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPR.

75. The method of any one of claims 45 to 74, wherein the targeting adaptor shares at least 95% identity to:
(SEQ ID NO: 59)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMSES
KYGPPCPPCPGGRMALIVLGGVAGLLLFIGLGIFF
CVRCRHRRRQ.

76. The method of any one of claims 45 to 74, wherein the targeting adaptor shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

77. The method of any one of claims 45 to 74, wherein the targeting adaptor shares at least 95% identity to:
(SEQ ID NO: 60)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCPGGRMALIVLGG
VAGLLLFIGLGIFFCVRCRHRRRQ.

78. The method of any one of claims 45 to 74, wherein the targeting adaptor shares at least 95% identity to:
(SEQ ID NO: 61)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCP.

79. The method of any one of claims 45 to 78, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen, CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gp120 or a fragment thereof.

80. The method of claim 79, wherein the cell-surface antigen is CD19.

81. The method of claim 80, wherein the tLBD-1 shares at least 95% identity to:
(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVSS.

82. A targeting adaptor, comprising a first ligand-binding domain (tLBD-1) specific for a cell-surface antigen and a second ligand-binding domain (tLBD-2) specific for a moiety select from folate, a CA9 ligand, fluorescein, and a derivative thereof.

83. The targeting adaptor of claim 82, wherein the tLBD-1 comprises an antibody or antigen-binding fragment thereof, optionally a single-chain variable fragment (scFv).

84. The targeting adaptor of claim 82, wherein the tLBD-2 comprises an antibody or antigen-binding fragment thereof, optionally a single-chain variable fragment (scFv).

85. The targeting adaptor of any one of claims 82 to 84, wherein the tLBD-2 comprises a folate receptor domain.

86. The targeting adaptor of claim 85, wherein the folate receptor domain is a folate receptor alpha (FRα) domain.

87. The targeting adaptor of claim 86, wherein the FRα domain shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

88. The targeting adaptor of any one of claims 82 to 84, wherein the tLBD-2 comprises a carbonic anhydrase IX (CA9) domain.

89. The targeting adaptor of claim 88, wherein the CA9 domain shares at least 95% identity to:
(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

90. The targeting adaptor of claim 88, wherein the CA9 domain shares at least 95% identity to:
(SEQ ID NO: 20)
HWRYGGDPPWPRVSPACAGRFQSPVDIRPQLAAFS
PALRPLELSGFQLPPLPELRLRNNGHSVQLTLPPG
LEMKLGPGREYRALQLHLHWGAAGRPGSEHTVEGH
RFPAEIHVVHLSTKYARVDEALGRPGGLAVLAAFL
EEGPEENSAYEQLLSRLEEIAEEGSETQVPGLDIS
ALLPSDFSRYFQYEGSLTTPPCAQGVIWTVFNQTV
SLSAKQLHTLSDTLWGPGDSRLQLNFRATQPLNGR
VIEASFPAGVD.

91. A polynucleotide encoding the targeting adaptor of any one of claims 82 to 90.

92. A vector comprising the polynucleotide of claim 91.

93. A kit comprising the system of any one of claims 1 to 37 and instructions for use.

94. The kit of claim 93, comprising the engineered immune cell comprising the chimeric receptor.

95. The kit of claim 93, comprising the vector encoding the chimeric receptor.

96. A vector for use with a gating adaptor, comprising:
(a) a polynucleotide encoding a targeting adaptor; and
(b) a polynucleotide encoding a chimeric receptor, optionally a chimeric antigen receptor,
wherein the targeting adaptor comprises a first ligand-binding domain (tLBD-1) specific for a cell-surface antigen and a second ligand-binding domain (tLBD-2) specific for the gating adaptor; and
wherein the chimeric receptor comprises an extracellular ligand-binding domain (rLBD) specific for the gating adaptor, a transmembrane domain, and an intracellular actuator domain.

97. The vector of claim 96, wherein the vector is a viral vector.

98. The vector of claim 97, wherein the viral vector is a retroviral vector.

99. The vector of claim 98, wherein the retroviral vector is a lentiviral vector.

100. The vector of claim 98, wherein the retroviral vector is a gamma-retroviral vector.

101. The vector of any one of claims 97 to 100, wherein the viral vector comprises a VSV G protein or functional variant thereof.

102. The vector of any one of claims 97 to 100, wherein the viral vector comprises a Cocal G protein or functional variant thereof.

103. The vector of claim 96, wherein the rLBD comprises an antibody or antigen-binding fragment thereof.

104. The vector of claim 103, wherein the rLBD comprises a single-chain variable fragment (scFv).

105. The vector of claim 96, wherein the rLBD comprises a T-cell receptor (TCR) or antigen-binding fragment thereof.

106. The vector of any one of claims 96 to 105, wherein the tLBD-1 comprises an antibody or antigen-binding fragment thereof.

107. The vector of claim 106, wherein the tLBD-1 comprises a single-chain variable fragment (scFv).

108. The vector of any one of claims 96 to 105, wherein the tLBD-1 comprises a T-cell receptor (TCR) or antigen-binding fragment thereof.

109. The vector of any one of claims 96 to 108, wherein the tLBD-2 comprises an antibody or antigen-binding fragment thereof.

110. The vector of any one of claims 96 to 108, wherein the tLBD-2 comprises a folate receptor domain.

111. The vector of claim 110, wherein the folate receptor domain is a folate receptor alpha (FRα) domain.

112. The vector of claim 111, wherein the FRα domain shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

113. The vector of any one of claims 96 to 108, wherein the tLBD-2 comprises a carbonic anhydrase IX (CA9) domain.

114. The vector of claim 113, wherein the CA9 domain shares at least 95% identity to:
(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

115. The vector of claim 113, wherein the CA9 domain shares at least 95% identity to:
(SEQ ID NO: 20)
HWRYGGDPPWPRVSPACAGRFQSPVDIRPQLAAFS
PALRPLELSGFQLPPLPELRLRNNGHSVQLTLPPG
LEMKLGPGREYRALQLHLHWGAAGRPGSEHTVEGH
RFPAEIHVVHLSTKYARVDEALGRPGGLAVLAAFL
EEGPEENSAYEQLLSRLEEIAEEGSETQVPGLDIS
ALLPSDFSRYFQYEGSLTTPPCAQGVIWTVFNQTV
SLSAKQLHTLSDTLWGPGDSRLQLNFRATQPLNGR
VIEASFPAGVD.

116. The vector of any one of claims 96 to 115, wherein the gating adaptor is a small molecule.

117. The vector of claim 116, wherein the gating adaptor comprises a first moiety recognized by rLBD and a second moiety recognized by tLBD-2.

118. The vector of claim 117, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.

119. The vector of claim 118, wherein the first moiety is fluorescein or a derivative thereof.

120. The vector of any one of claims 117 to 119, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.

121. The vector of claim 120, wherein the second moiety is folate or a derivative thereof.

122. The vector of claim 120, wherein the second moiety is a CA9 ligand or a derivative thereof.

123. The vector of claim 121, wherein the gating adaptor is a folate-fluorescein conjugate.

124. The vector of claim 120, wherein the gating adaptor is a CA9 ligand-fluorescein conjugate.

125. The vector of any one of claims 96 to 124, wherein the chimeric receptor comprises one polypeptide chain.

126. The vector of any one of claims 96 to 124, wherein the chimeric receptor comprises at least two polypeptide chains.

127. The vector of any one of claims 96 to 126, wherein the chimeric receptor specifically binds fluorescein.

128. The vector of claim 127, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.

129. The vector of claim 127, wherein the rLBD comprises a polypeptide sequence sharing at least 95% identity to:
(SEQ ID NO: 31)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLG;
and a polypeptide sequence sharing
at least 95% identity to:
(SEQ ID NO: 32)
QVQLVESGGNLVQPGGSLRLSCAASGFTFGSFSMS
WVRQAPGGGLEWVAGLSARSSLTHYADSVKGRFTI
SRDNAKNSVYLQMNSLRVEDTAVYYCARRSYDSSG
YWGHFYSYMDVWGQGTLVTVSS.

130. The vector of claim 129, wherein the rLBD shares at least 95% identity to:
(SEQ ID NO: 30)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSS.

131. The vector of claim 130, wherein the chimeric receptor shares at least 95% identity to:
(SEQ ID NO: 58)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSSESKYGPPCPPCPMFWVLVVVGGVLAC
YSLLVTVAFIIFWVKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPR.

132. The vector of any one of claims 96 to 131, wherein the targeting adaptor shares at least 95% identity to:
(SEQ ID NO: 59)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMSES
KYGPPCPPCPGGRMALIVLGGVAGLLLFIGLGIFF
CVRCRHRRRQ.

133. The vector of any one of claims 96 to 131, wherein the targeting adaptor shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

134. The vector of any one of claims 96 to 131, wherein the targeting adaptor shares at least 95% identity to:
(SEQ ID NO: 60)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCPGGRMALIVLGG
VAGLLLFIGLGIFFCVRCRHRRRQ.

135. The vector of any one of claims 96 to 131, wherein the targeting adaptor shares at least 95% identity to:
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRF
(SEQ ID NO: 61)
PAEIHVVHLSTAFARVDEALGRPGGLAVLAAFLEE
GPEENSAYEQLLSRLEEIAEEGSETQVPGLDISAL
LPSDFSRYFQYEGSLTTPPCAQGVIWTVFNQTVML
SAKQLHTLSDTLWGPGDSRLQLNFRATQPLNGRVI
EASFPAGVDESKYGPPCPPCP.

136. The vector of any one of claims 96 to 135, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gp120 or a fragment thereof.

137. The vector of claim 136, wherein the cell-surface antigen is CD19.

138. The vector of claim 137, wherein the tLBD-1 shares at least 95% identity to:
(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVSS.

139. An isolated cell, comprising:
(a) a polynucleotide encoding a targeting adaptor, and/or a targeting adaptor comprising a transmembrane domain and expressed on the surface of the isolated cell; and
(b) a polynucleotide encoding a chimeric receptor, optionally a chimeric antigen receptor, and/or a chimeric receptor expressed on the surface of the isolated cell,
wherein the targeting adaptor comprises a first ligand-binding domain (tLBD-1) specific for a cell-surface antigen and a second ligand-binding domain (tLBD-2) specific for a gating adaptor; and
wherein the chimeric receptor comprises an extracellular ligand-binding domain (rLBD) specific for the gating adaptor, a transmembrane domain, and an intracellular actuator domain.

140. The cell of claim 139, wherein the rLBD comprises an antibody or antigen-binding fragment thereof.

141. The cell of claim 140, wherein the rLBD comprises a single-chain variable fragment (scFv).

142. The cell of claim 139, wherein the rLBD comprises a T-cell receptor (TCR) or antigen-binding fragment thereof.

143. The cell of any one of claims 139 to 142, wherein the tLBD-1 comprises an antibody or antigen-binding fragment thereof.

144. The cell of claim 143, wherein the tLBD-1 comprises a single-chain variable fragment (scFv).

145. The cell of any one of claims 139 to 142, wherein the tLBD-1 comprises a T-cell receptor (TCR) or antigen-binding fragment thereof.

146. The cell of any one of claims 139 to 145, wherein the tLBD-2 comprises an antibody or antigen-binding fragment thereof.

147. The cell of any one of claims 139 to 145, wherein the tLBD-2 comprises a folate receptor domain.

148. The cell of claim 147, wherein the folate receptor domain is a folate receptor alpha (FRα) domain.

149. The cell of claim 148, wherein the FRα domain shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMS.

150. The cell of any one of claims 139 to 145, wherein the tLBD-2 comprises a carbonic anhydrase IX (CA9) domain.

151. The cell of claim 150, wherein the CA9 domain shares at least 95% identity to:
(SEQ ID NO: 19)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVD.

152. The cell of claim 150, wherein the CA9 domain shares at least 95% identity to:
(SEQ ID NO: 20)
HWRYGGDPPWPRVSPACAGRFQSPVDIRPQLAAFS
PALRPLELSGFQLPPLPELRLRNNGHSVQLTLPPG
LEMKLGPGREYRALQLHLHWGAAGRPGSEHTVEGH
RFPAEIHVVHLSTKYARVDEALGRPGGLAVLAAFL
EEGPEENSAYEQLLSRLEEIAEEGSETQVPGLDIS
ALLPSDFSRYFQYEGSLTTPPCAQGVIWTVFNQTV
SLSAKQLHTLSDTLWGPGDSRLQLNFRATQPLNGR
VIEASFPAGVD.

153. The cell of any one of claims 139 to 152, wherein the gating adaptor is a small molecule.

154. The cell of claim 153, wherein the gating adaptor comprises a first moiety recognized by rLBD and a second moiety recognized by tLBD-2.

155. The cell of claim 154, wherein the first moiety is folate, fluorescein, acetazolamide, a CA9 ligand, tacrolimus, rapamycin, a rapalog, a CD28 ligand, poly(his) tag, Strep-tag, FLAG-tag, VS-tag, Myc-tag, HA-tag, NE-tag, biotin, digoxigenin, dinitrophenol, or a derivative thereof.

156. The cell of claim 155, wherein the first moiety is fluorescein or a derivative thereof.

157. The cell of any one of claims 154 to 156, wherein the second moiety is folate, acetazolamide, a CA9 ligand, fluorescein, tacrolimus, rapamycin, a rapalog, CD28 ligand, poly(his) tag, Strep-tag, FLAG-tag, VS-tag, Myc-tag, HA-tag, NE-tag, biotin, digoxigenin, dinitrophenol, or a derivative thereof.

158. The cell of claim 157, wherein the second moiety is folate or a derivative thereof.

159. The cell of claim 157, wherein the second moiety is a CA9 ligand or a derivative thereof.

160. The cell of claim 158, wherein the gating adaptor is a folate-fluorescein conjugate.

161. The cell of claim 157, wherein the gating adaptor is a CA9 ligand-fluorescein conjugate.

162. The cell of any one of claims 139 to 161, wherein the chimeric receptor comprises one polypeptide chain.

163. The cell of any one of claims 139 to 161, wherein the chimeric receptor comprises at least two polypeptide chains.

164. The cell of any one of claims 139 to 163, wherein the chimeric receptor specifically binds fluorescein.

165. The cell of claim 164, wherein the rLBD comprises CDRL1, CDRL2, CDRL3, CDRH1, CDRH2, and CDRH3 sequences of an scFv sequence according to (i) SEQ ID NO: 2, (ii) SEQ ID NO: 30, (iii) SEQ ID NO: 33, or any one of SEQ ID NOs: 99-104.

166. The cell of claim 164, wherein the rLBD comprises a polypeptide sequence sharing at least 95% identity to:
(SEQ ID NO: 31)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLG;
and a polypeptide sequence sharing at least 95% identity to:
(SEQ ID NO: 32)
QVQLVESGGNLVQPGGSLRLSCAASGFTFGSFSMS
WVRQAPGGGLEWVAGLSARSSLTHYADSVKGRFTI
SRDNAKNSVYLQMNSLRVEDTAVYYCARRSYDSSG
YWGHFYSYMDVWGQGTLVTVSS.

167. The cell of claim 166, wherein the rLBD shares at least 95% identity to:
(SEQ ID NO: 30)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSS.

168. The cell of claim 167, wherein the chimeric receptor shares at least 95% identity to:
(SEQ ID NO: 58)
SVLTQPSSVSAAPGQKVTISCSGSTSNIGNNYVSW
YQQHPGKAPKLMIYDVSKRPSGVPDRFSGSKSGNS
ASLDISGLQSEDEADYYCAAWDDSLSEFLFGTGTK
LTVLGSTSGSGKPGSGEGSTKGQVQLVESGGNLVQ
PGGSLRLSCAASGFTFGSFSMSWVRQAPGGGLEWV
AGLSARSSLTHYADSVKGRFTISRDNAKNSVYLQM
NSLRVEDTAVYYCARRSYDSSGYWGHFYSYMDVWG
QGTLVTVSSESKYGPPCPPCPMFWVLVVVGGVLAC
YSLLVTVAFIIFWVKRGRKKLLYIFKQPFMRPVQT
TQEEDGCSCRFPEEEEGGCELRVKFSRSADAPAYQ
QGQNQLYNELNLGRREEYDVLDKRRGRDPEMGGKP
RRKNPQEGLYNELQKDKMAEAYSEIGMKGERRRGK
GHDGLYQGLSTATKDTYDALHMQALPPR.

169. The cell of any one of claims 139 to 168, wherein the targeting adaptor shares at least 95% identity to:
(SEQ ID NO: 59)
GSSRTELLNVCMNAKHHKEKPGPEDKLHEQCRPWR
KNACCSTNTSQEAHKDVSYLYRFNWNHCGEMAPAC
KRHFIQDTCLYECSPNLGPWIQQVDQSWRKERVLN
VPLCKEDCEQWWEDCRTSYTCKSNWHKGWNWTSGF
NKCAVGAACQPFHFYFPTPTVLCNEIWTHSYKVSN
YSRGSGRCIQMWFDPAQGNPNEEVARFYAAAMSES
KYGPPCPPCPGGRMALIVLGGVAGLLLFIGLGIFF
CVRCRHRRRQ.

170. The cell of any one of claims 139 to 168, wherein the targeting adaptor shares at least 95% identity to:
(SEQ ID NO: 18)
GSSRTELLNVCMNAKHHKEKPGPEDKLHEQCRPWR
KNACCSTNTSQEAHKDVSYLYRFNWNHCGEMAPAC
KRHFIQDTCLYECSPNLG
PWIQQVDQSWRKERVLNVPLCKEDCEQWWEDCRTS
YTCKSNWHKGWNWTSGFNKCAVGAACQPFHFYFPT
PTVLCNEIWTHSYKVSNYSRGSGRCIQMWFDPAQG
NPNEEVARFYAAAMS.

171. The cell of any one of claims 139 to 168, wherein the targeting adaptor shares at least 95% identity to:
(SEQ ID NO: 60)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCPGGRMALIVLGG
VAGLLLFIGLGIFFCVRCRHRRRQ.

172. The cell of any one of claims 139 to 168, wherein the targeting adaptor shares at least 95% identity to:
(SEQ ID NO: 61)
HHWRYGGDPPWPRVSPACAGRFQSPVDIRPQLAAF
SPALRPLELLGFQLPPLPELRLRNNGHSVQLTLPP
GLEMALGPGREYRALQLHLHWGAAGRPGSEHTVEG
HRFPAEIHVVHLSTAFARVDEALGRPGGLAVLAAF
LEEGPEENSAYEQLLSRLEEIAEEGSETQVPGLDI
SALLPSDFSRYFQYEGSLTTPPCAQGVIWTVFNQT
VMLSAKQLHTLSDTLWGPGDSRLQLNFRATQPLNG
RVIEASFPAGVDESKYGPPCPPCP.

173. The cell of any one of claims 139 to 172, wherein the cell-surface antigen is CD19, ABT-806, CD3, CD28, CD134, CD137, folate receptor, 4-1BB, PD1, CD45, CD8a, CD4, CD8, CD4, LAG3, CD3e, CD69, CD45RA, CD62L, CD45RO, CD62F, CD95, 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD20, CD22, CD23, CD24, CD25, CD30, CD33, CD34, CD40, CD44, CD56, CLL-1, c-Met, CMV-specific antigen, CS-1, CSPG4, CTLA-4, DLL3, disialoganglioside GD2, ductal-epithelial mucine, EBV-specific antigen, EGFR, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (FDVTW-MAA), HIV-1 envelope glycoprotein gp41, HPV-specific antigen, human telomerase reverse transcriptase, IGFI receptor, IGF-II, IL-1 1Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin, the Al domain of tenascin-C (TnC Al), thyroglobulin, tumor stromal antigens, vascular endothelial growth factor receptor-2 (VEGFR2), HIV gpl20 or a fragment thereof.

174. The cell of claim 173, wherein the cell-surface antigen is CD19.

175. The cell of claim 174, wherein the tLBD-1 shares at least 95% identity to:
(SEQ ID NO: 52)
DIQMTQTTSSLSASLGDRVTISCRASQDISKYLNW
YQQKPDGTVKLLIYHTSRLHSGVPSRFSGSGSGTD
YSLTISNLEQEDIATYFCQQGNTLPYTFGGGTKLE
ITGSTSGSGKPGSGEGSTKGEVKLQESGPGLVAPS
QSLSVTCTVSGVSLPDYGVSWIRQPPRKGLEWLGV
IWGSETTYYNSALKSRLTIIKDNSKSQVFLKMNSL
QTDDTAIYYCAKHYYYGGSYAMDYWGQGTSVTVSS.
